SEQUENCE LISTING (133 sequences)

SEQ ID NO: 1 - DNA, 1155 bp, Homo sapiens
atggcagcccagaatggaaacaccagtttcacacccaactttaatccaccccaagaccatgcctcctccctctcctttaacttcagttatggtgattatgacctccctatggatgaggatgaggacatgaccaagacccggaccttcttcgcagccaagatcgtcattggcattgcactggcaggcatcatgctggtctgcggcatcggtaactttgtctttatcgctgccctcacccgctataagaagttgcgcaacctcaccaatctgctcattgccaacctggccatctccgacttcctggtggccatcatctgctgccccttcgagatggactactacgtggtacggcagctctcctgggagcatggccacgtgctctgtgcctccgtcaactacctgcgcaccgtctccctctacgtctccaccaatgccttgctggccattgccattgacagatatctcgccatcgttcaccccttgaaaccacggatgaattatcaaacggcctccttcctgatcgccttggtctggatggtgtccattctcattgccatcccatcggcttactttgcaacagaaacggtcctctttattgtcaagagccaggagaagatcttctgtggccagatctggcctgtggatcagcagctctactacaagtcctacttcctcttcatctttggtgtcgagttcgtgggccctgtggtcaccatgaccctgtgctatgccaggatctcccgggagctctggttcaaggcagtccctgggttccagacggagcagattcgcaagcggctgcgctgccgcaggaagacggtcctggtgctcatgtgcattctcacggcctatgtgctgtgctgggcacccttctacggtttcaccatcgttcgtgacttcttccccactgtgttcgtgaaggaaaagcactacctcactgccttctacgtggtcgagtgcatcgccatgagcaacagcatgatcaacaccgtgtgcttcgtgacggtcaagaacaacaccatgaagtacttcaagaagatgatgctgctgcactggcgtccctcccagcgggggagcaagtccagtgctgaccttgacctcagaaccaacggggtgcccaccacagaagaggtggactgtatcaggctgaagtga

SEQ ID NO: 2 - protein, 384 aa, Homo sapiens
MAAQNGNTSFTPNFNPPQDHASSLSFNFSYGDYDLPMDEDEDMTKTRTFFAAKIVIGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQRGSKSSADLDLRTNGVPTTEEVDCIRLK

SEQ ID NO: 3 - DNA, 1260 bp, Homo sapiens
atgctggcagctgcctttgcagactctaactccagcagcatgaatgtgtcctttgctcacctccactttgccggagggtacctgccctctgattcccaggactggagaaccatcatcccggctctcttggtggctgtctgcctggtgggcttcgtgggaaacctgtgtgtgattggcatcctccttcacaatgcttggaaaggaaagccatccatgatccactccctgattctgaatctcagcctggctgatctctccctcctgctgttttctgcacctatccgagctacggcgtactccaaaagtgtttgggatctaggctggtttgtctgcaagtcctctgactggtttatccacacatgcatggcagccaagagcctgacaatcgttgtggtggccaaagtatgcttcatgtatgcaagtgacccagccaagcaagtgagtatccacaactacaccatctggtcagtgctggtggccatctggactgtggctagcctgttacccctgccggaatggttctttagcaccatcaggcatcatgaaggtgtggaaatgtgcctcgtggatgtaccagctgtggctgaagagtttatgtcgatgtttggtaagctctacccactcctggcatttggccttccattattttttgccagcttttatttctggagagcttatgaccaatgtaaaaaacgaggaactaagactcaaaatcttagaaaccagatacgctcaaagcaagtcacagtgatgctgctgagcattgccatcatctctgctctcttgtggctccccgaatgggtagcttggctgtgggtatggcatctgaaggctgcaggcccggccccaccacaaggtttcatagccctgtctcaagtcttgatgttttccatctcttcagcaaatcctctcatttttcttgtgatgtcggaagagttcagggaaggcttgaaaggtgtatggaaatggatgataaccaaaaaacctccaactgtctcagagtctcaggaaacaccagctggcaactcagagggtcttcctgacaaggttccatctccagaatccccagcatccataccagaaaaagagaaacccagctctccctcctctggcaaagggaaaactgagaaggcagagattcccatccttcctgacgtagagcagttttggcatgagagggacacagtcccttctgtacaggacaatgaccctatcccctgggaacatgaagatcaagagacaggggaaggtgttaaatag

SEQ ID NO: 4 - protein, 419 aa, Homo sapiens
MLAAAFADSNSSSMNVSFAHLHFAGGYLPSDSQDWRTIIPALLVAVCLVGFVGNLCVIGILLHNAWKGKPSMIHSLILNLSLADLSLLLFSAPIRATAYSKSVWDLGWFVCKSSDWFIHTCMAAKSLTIVVVAKVCFMYASDPAKQVSIHNYTIWSVLVAIWTVASLLPLPEWFFSTIRHHEGVEMCLVDVPAVAEEFMSMFGKLYPLLAFGLPLFFASFYFWRAYDQCKKRGTKTQNLRNQIRSKQVTVMLLSIAIISALLWLPEWVAWLWVWHLKAAGPAPPQGFIALSQVLMFSISSANPLIFLVMSEEFREGLKGVWKWMITKKPPTVSESQETPAGNSEGLPDKVPSPESPASIPEKEKPSSPSSGKGKTEKAEIPILPDVEQFWHERDTVPSVQDNDPIPWEHEDQETGEGVK

SEQ ID NO: 5 - DNA, 1014 bp, Homo sapiens
atggggaacgattctgtcagctacgagtatggggattacagcgacctctcggaccgccctgtggactgcctggatggcgcctgcctggccatcgacccgctgcgcgtggccccgctcccactgtatgccgccatcttcctggtgggggtgccgggcaatgccatggtggcctgggtggctgggaaggtggcccgccggagggtgggtgccacctggttgctccacctggccgtggcggatttgctgtgctgtttgtctctgcccatcctggcagtgcccattgcccgtggaggccactggccgtatggtgcagtgggctgtcgggcgctgccctccatcatcctgctgaccatgtatgccagcgtcctgctcctggcagctctcagtgccgacctctgcttcctggctctcgggcctgcctggtggtctacggttcagcgggcgtgcggggtgcaggtggcctgtggggcagcctggacactggccttgctgctcaccgtgccctccgccatctaccgccggctgcaccaggagcacttcccagcccggctgcagtgtgtggtggactacggcggctcctccagcaccgagaatgcggtgactgccatccggtttctttttggcttcctggggcccctggtggccgtggccagctgccacagtgccctcctgtgctgggcagcccgacgctgccggccgctgggcacagccattgtggtggggttttttgtctgctgggcaccctaccacctgctggggctggtgctcactgtggcggccccgaactccgcactcctggccagggccctgcgggctgaacccctcatcgtgggccttgccctcgctcacagctgcctcaatcccatgctcttcctgtattttgggagggctcaactccgccggtcactgccagctgcctgtcactgggccctgagggagtcccagggccaggacgaaagtgtggacagcaagaaatccaccagccatgacctggtctcggagatggaggtgtag

SEQ ID NO: 6 - protein, 337 aa, Homo sapiens [sequence omitted]

SEQ ID NO: 7 - DNA, 1272 bp, Homo sapiens
atgttgtgtcaccgtggtggccagctgatagtgccaatcatcccactttgccctgagcactcctgcaggggtagaagactccagaaccttctctcaggcccatggcccaagcagcccatggaacttcataacctgagctctccatctccctctctctcctcctctgttctccctccctccttctctccctcaccctcctctgctccctctgcctttaccactgtgggggggtcctctggagggccctgccaccccacctcttcctcgctggtgtctgccttcctggcaccaatcctggccctggagtttgtcctgggcctggtggggaacagtttggccctcttcatcttctgcatccacacgcggccctggacctccaacacggtgttcctggtcagcctggtggccgctgacttcctcctgatcagcaacctgcccctccgcgtggactactacctcctccatgagacctggcgctttggggctgctgcctgcaaagtcaacctcttcatgctgtccaccaaccgcacggccagcgttgtcttcctcacagccatcgcactcaaccgctacctgaaggtggtgcagccccaccacgtgctgagccgtgcttccgtgggggcagctgcccgggtggccgggggactctgggtgggcatcctgctcctcaacgggcacctgctcctgagcaccttctccggcccctcctgcctcagctacagggtgggcacgaagccctcggcctcgctccgctggcaccaggcactgtacctgctggagttcttcctgccactggcgctcatcctctttgctattgtgagcattgggctcaccatccggaaccgtggtctgggcgggcaggcaggcccgcagagggccatgcgtgtgctggccatggtggtggccgtctacaccatctgcttcttgcccagcatcatctttggcatggcttccatggtggctttctggctgtccgcctgccgatccctggacctctgcacacagctcttccatggctccctggccttcacctacctcaacagtgtcctggaccccgtgctctactgcttctctagccccaacttcctccaccagagccgggccttgctgggcctcacgcggggccggcagggcccagtgagcgacgagagctcctaccaaccctccaggcagtggcgctaccgggaggcctctaggaaggcggaggccatagggaagctgaaagtgcagggcgaggtctctctggaaaaggaaggctcctcccagggctga

SEQ ID NO: 8 - protein, 423 aa, Homo sapiens
MLCHRGGQLIVPIIPLCPEHSCRGRRLQNLLSGPWPKQPMELHNLSSPSPSLSSSVLPPSFSPSPSSAPSAFTTVGGSSGGPCHPTSSSLVSAFLAPILALEFVLGLVGNSLALFIFCIHTRPWTSNTVFLVSLVAADFLLISNLPLRVDYYLLHETWRFGAAACKVNLFMLSTNRTASVVFLTAIALNRYLKVVQPHHVLSRASVGAAARVAGGLWVGILLLNGHLLLSTFSGPSCLSYRVGTKPSASLRWHQALYLLEFFLPLALILFAIVSIGLTIRNRGLGGQAGPQRAMRVLAMVVAVYTICFLPSIIFGMASMVAFWLSACRSLDLCTQLFHGSLAFTYLNSVLDPVLYCFSSPNFLHQSRALLGLTRGRQGPVSDESSYQPSRQWRYREASRKAEAIGKLKVQGEVSLEKEGSSQG

SEQ ID NO: 9 - DNA, 966 bp, Homo sapiens
atgaaccagactttgaatagcagtgggaccgtggagtcagccctaaactattccagagggagcacagtgcacacggcctacctggtgctgagctccctggccatgttcacctgcctgtgcgggatggcaggcaacagcatggtgatctggctgctgggctttcgaatgcacaggaaccccttctgcatctatatcctcaacctggcggcagccgacctcctcttcctcttcagcatggcttccacgctcagcctggaaacccagcccctggtcaataccactgacaaggtccacgagctgatgaagagactgatgtactttgcctacacagtgggcctgagcctgctgacggccatcagcacccagcgctgtctctctgtcctcttccctatctggttcaagtgtcaccggcccaggcacctgtcagcctgggtgtgtggcctgctgtggacactctgtctcctgatgaacgggttgacctcttccttctgcagcaagttcttgaaattcaatgaagatcggtgcttcagggtggacatggtccaggccgccctcatcatgggggtcttaaccccagtgatgactctgtccagcctgaccctctttgtctgggtgcggaggagctcccagcagtggcggcggcagcccacacggctgttcgtggtggtcctggcctctgtcctggtgttcctcatctgttccctgcctctgagcatctactggtttgtgctctactggttgagcctgccgcccgagatgcaggtcctgtgcttcagcttgtcacgcctctcctcgtccgtaagcagcagcgccaaccccgtcatctacttcctggtgggcagccggaggagccacaggctgcccaccaggtccctggggactgtgctccaacaggcgcttcgcgaggagcccgagctggaaggtggggagacgcccaccgtgggcaccaatgagatgggggcttga

SEQ ID NO: 10 - protein, 321 aa, Homo sapiens
MNQTLNSSGTVESALNYSRGSTVHTAYLVLSSLAMFTCLCGMAGNSMVIWLLGFRMHRNPFCIYILNLAAADLLFLFSMASTLSLETQPLVNTTDKVHELMKRLMYFAYTVGLSLLTAISTQRCLSVLFPIWFKCHRPRHLSAWVCGLLWTLCLLMNGLTSSFCSKFLKFNEDRCFRVDMVQAALIMGVLTPVMTLSSLTLFVWVRRSSQQWRRQPTRLFVVVLASVLVFLICSLPLSIYWFVLYWLSLPPEMQVLCFSLSRLSSSVSSSANPVIYFLVGSRRSHRLPTRSLGTVLQQALREEPELEGGETPTVGTNEMGA

SEQ ID NO: 11 - DNA, 1356 bp, Homo sapiens
atggagtcctcacccatcccccagtcatcagggaactcttccactttggggagggtccctcaaaccccaggtccctctactgccagtggggtcccggaggtggggctacgggatgttgcttcggaatctgtggccctcttcttcatgctcctgctggacttgactgctgtggctggcaatgccgctgtgatggccgtgatcgccaagacgcctgccctccgaaaatttgtcttcgtcttccacctctgcctggtggacctgctggctgccctgaccctcatgcccctggccatgctctccagctctgccctctttgaccacgccctctttggggaggtggcctgccgcctctacttgtttctgagcgtgtgctttgtcagcctggccatcctctcggtgtcagccatcaatgtggagcgctactattacgtagtccaccccatgcgctacgaggtgcgcatgacgctggggctggtggcctctgtgctggtgggtgtgtgggtgaaggccttggccatggcttctgtgccagtgttgggaagggtctcctgggaggaaggagctcccagtgtccccccaggctgttcactccagtggagccacagtgcctactgccagctttttgtggtggtctttgctgtcctttactttctgttgcccctgctcctcatacttgtggtctactgcagcatgttccgagtggcccgcgtggctgccatgcagcacgggccgctgcccacgtggatggagacaccccggcaacgctccgaatctctcagcagccgctccacgatggtcaccagctcgggggccccccagaccaccccacaccggacgtttgggggagggaaagcagcagtggttctcctggctgtggggggacagttcctgctctgttggttgccctacttctctttccacctctatgttgccctgagtgctcagcccatttcaactgggcaggtggagagtgtggtcacctggattggctacttttgcttcacttccaaccctttcttctatggatgtctcaaccggcagatccggggggagctcagcaagcagtttgtctgcttcttcaagccagctccagaggaggagctgaggctgcctagccgggagggctccattgaggagaacttcctgcagttccttcaggggactggctgtccttctgagtcctgggtttcccgacccctacccagccccaagcaggagccacctgctgttgactttcgaatcccaggccagatagctgaggagacctctgagttcctggagcagcaactcaccagcgacatcatcatgtcagacagctacctccgtcctgccgcctcaccccggctggagtcatga

SEQ ID NO: 12 - protein, 451 aa, Homo sapiens
MESSPIPQSSGNSSTLGRVPQTPGPSTASGVPEVGLRDVASESVALFFMLLLDLTAVAGNAAVMAVIAKTPALRKFVFVFHLCLVDLLAALTLMPLAMLSSSALFDHALFGEVACRLYLFLSVCFVSLAILSVSAINVERYYYVVHPMRYEVRMTLGLVASVLVGVWVKALAMASVPVLGRVSWEEGAPSVPPGCSLQWSHSAYCQLFVVVFAVLYFLLPLLLILVVYCSMFRVARVAAMQHGPLPTWMETPRQRSESLSSRSTMVTSSGAPQTTPHRTFGGGKAAVVLLAVGGQFLLCWLPYFSFHLYVALSAQPISTGQVESVVTWIGYFCFTSNPFFYGCLNRQIRGELSKQFVCFFKPAPEEELRLPSREGSIEENFLQFLQGTGCPSESWVSRPLPSPKQEPPAVDFRIPGQIAEETSEFLEQQLTSDIIMSDSYLRPAASPRLES

SEQ ID NO: 13 - DNA, 1041 bp, Homo sapiens
atggagagaaaatttatgtccttgcaaccatccatctccgtatcagaaatggaaccaaatggcaccttcagcaataacaacagcaggaactgcacaattgaaaacttcaagagagaatttttcccaattgtatatctgataatatttttctggggagtcttgggaaatgggttgtccatatatgttttcctgcagccttataagaagtccacatctgtgaacgttttcatgctaaatctggccatttcagatctcctgttcataagcacgcttcccttcagggctgactattatcttagaggctccaattggatatttggagacctggcctgcaggattatgtcttattccttgtatgtcaacatgtacagcagtatttatttcctgaccgtgctgagtgttgtgcgtttcctggcaatggttcacccctttcggcttctgcatgtcaccagcatcaggagtgcctggatcctctgtgggatcatatggatccttatcatggcttcctcaataatgctcctggacagtggctctgagcagaacggcagtgtcacatcatgcttagagctgaatctctataaaattgctaagctgcagaccatgaactatattgccttggtggtgggctgcctgctgccatttttcacactcagcatctgttatctgctgatcattcgggttctgttaaaagtggaggtcccagaatcggggctgcgggtttctcacaggaaggcactgaccaccatcatcatcaccttgatcatcttcttcttgtgtttcctgccctatcacacactgaggaccgtccacttgacgacatggaaagtgggtttatgcaaagacagactgcataaagctttggttatcacactggccttggcagcagccaatgcctgcttcaatcctctgctctattactttgctggggagaattttaaggacagactaaagtctgcactcagaaaaggccatccacagaaggcaaagacaaagtgtgttttccctgttagtgtgtggttgagaaaggaaacaagagtataa

SEQ ID NO: 14 - protein, 346 aa, Homo sapiens
MERKFMSLQPSISVSEMEPNGTFSNNNSRNCTIENFKREFFPIVYLIIFFWGVLGNGLSIYVFLQPYKKSTSVNVFMLNLAISDLLFISTLPFRADYYLRGSNWIFGDLACRIMSYSLYVNMYSSIYFLTVLSVVRFLAMVHPFRLLHVTSIRSAWILCGIIWILIMASSIMLLDSGSEQNGSVTSCLELNLYKIAKLQTMNYIALVVGCLLPFFTLSICYLLIIRVLLKVEVPESGLRVSHRKALTTIIITLIIFFLCFLPYHTLRTVHLTTWKVGLCKDRLHKALVITLALAAANACFNPLLYYFAGENFKDRLKSALRKGHPQKAKTKCVFPVSVWLRKETRV

SEQ ID NO: 15 - DNA, 1527 bp, Homo sapiens
atgacgtccacctgcaccaacagcacgcgcgagagtaacagcagccacacgtgcatgcccctctccaaaatgcccatcagcctggcccacggcatcatccgctcaaccgtgctggttatcttcctcgccgcctctttcgtcggcaacatagtgctggcgctagtgttgcagcgcaagccgcagctgctgcaggtgaccaaccgttttatctttaacctcctcgtcaccgacctgctgcagatttcgctcgtggccccctgggtggtggccacctctgtgcctctcttctggcccctcaacagccacttctgcacggccctggttagcctcacccacctgttcgccttcgccagcgtcaacaccattgtcgtggtgtcagtggatcgctacttgtccatcatccaccctctctcctacccgtccaagatgacccagcgccgcggttacctgctcctctatggcacctggattgtggccatcctgcagagcactcctccactctacggctggggccaggctgcctttgatgagcgcaatgctctctgctccatgatctggggggccagccccagctacactattctcagcgtggtgtccttcatcgtcattccactgattgtcatgattgcctgctactccgtggtgttctgtgcagcccggaggcagcatgctctgctgtacaatgtcaagagacacagcttggaagtgcgagtcaaggactgtgtggagaatgaggatgaagagggagcagagaagaaggaggagttccaggatgagagtgagtttcgccgccagcatgaaggtgaggtcaaggccaaggagggcagaatggaagccaaggacggcagcctgaaggccaaggaaggaagcacggggaccagtgagagtagtgtagaggccaggggcagcgaggaggtcagagagagcagcacggtggccagcgacggcagcatggagggtaaggaaggcagcaccaaagttgaggagaacagcatgaaggcagacaagggtcgcacagaggtcaaccagtgcagcattgacttgggtgaagatgacatggagtttggtgaagacgacatcaatttcagtgaggatgacgtcgaggcagtgaacatcccggagagcctcccacccagtcgtcgtaacagcaacagcaaccctcctctgcccaggtgctaccagtgcaaagctgctaaagtgatcttcatcatcattttctcctatgtgctatccctggggccctactgctttttagcagtcctggccgtgtgggtggatgtcgaaacccaggtaccccagtgggtgatcaccataatcatctggcttttcttcctgcagtgctgcatccacccctatgtctatggctacatgcacaagaccattaagaaggaaatccaggacatgctgaagaagttcttctgcaaggaaaagcccccgaaagaagatagccacccagacctgcccggaacagagggtgggactgaaggcaagattgtcccttcctacgattctgctacttttccttga

SEQ ID NO: 16 - protein, 508 aa, Homo sapiens
MTSTCTNSTRESNSSHTCMPLSKMPISLAHGIIRSTVLVIFLAASFVGNIVLALVLQRKPQLLQVTNRFIFNLLVTDLLQISLVAPWVVATSVPLFWPLNSHFCTALVSLTHLFAFASVNTIVVVSVDRYLSIIHPLSYPSKMTQRRGYLLLYGTWIVAILQSTPPLYGWGQAAFDERNALCSMIWGASPSYTILSVVSFIVIPLIVMIACYSVVFCAARRQHALLYNVKRHSLEVRVKDCVENEDEEGAEKKEEFQDESEFRRQHEGEVKAKEGRMEAKDGSLKAKEGSTGTSESSVEARGSEEVRESSTVASDGSMEGKEGSTKVEENSMKADKGRTEVNQCSIDLGEDDMEFGEDDINFSEDDVEAVNIPESLPPSRRNSNSNPPLPRCYQCKAAKVIFIIIFSYVLSLGPYCFLAVLAVWVDVETQVPQWVITIIIWLFFLQCCIHPYVYGYMHKTIKKEIQDMLKKFFCKEKPPKEDSHPDLPGTEGGTEGKIVPSYDSATFP

SEQ ID NO: 17 - DNA, 1068 bp, Homo sapiens
atgcccttgacggacggcatttcttcatttgaggacctcttggctaacaatatcctcagaatatttgtctgggttatagctttcattacctgctttggaaatctttttgtcattggcatgagatctttcattaaagctgaaaatacaactcacgctatgtccatcaaaatcctttgttgcgctgattgcctgatgggtgtttacttgttctttgttggcattttcgatataaaataccgagggcagtatcagaagtatgccttgctgtggatggagagcgtgcagtgccgcctcatggggttcctggccatgctgtccaccgaagtctctgttctgctactgacctacttgactttggagaagttcctggtcattgtcttccccttcagtaacattcgacctggaaaacggcagacctcagtcatcctcatttgcatctggatggcgggatttttaatagctgtaattccattttggaataaggattattttggaaacttttatgggaaaaatggagtatgtttcccactttattatgaccaaacagaagatattggaagcaaagggtattctcttggaattttcctaggtgtgaacttgctggcttttctcatcattgtgttttcctatattactatgttctgttccattcaaaaaaccgccttgcagaccacagaagtaaggaattgttttggaagagaggtggctgttgcaaatcgtttcttttttatagtgttctctgatgccatctgctggattcctgtatttgtagttaaaatcctttccctcttccgggtggaaataccagacacaatgacttcctggatagtgatttttttccttccagttaacagtgctttgaatccaatcctctatactctcacaaccaacttttttaaggacaagttgaaacagctgctgcacaaacatcagaggaaatcaattttcaaaattaaaaaaaaaagtttatctacatccattgtgtggatagaggactcctcttccctgaaacttggggttttgaacaaaataacacttggagacagtataatgaaaccagtttcctag

SEQ ID NO: 18 - protein, 355 aa, Homo sapiens
MPLTDGISSFEDLLANNILRIFVWVIAFITCFGNLFVIGMRSFIKAENTTHAMSIKILCCADCLMGVYLFFVGIFDIKYRGQYQKYALLWMESVQCRLMGFLAMLSTEVSVLLLTYLTLEKFLVIVFPFSNIRPGKRQTSVILICIWMAGFLIAVIPFWNKDYFGNFYGKNGVCFPLYYDQTEDIGSKGYSLGIFLGVNLLAFLIIVFSYITMFCSIQKTALQTTEVRNCFGREVAVANRFFFIVFSDAICWIPVFVVKILSLFRVEIPDTMTSWIVIFFLPVNSALNPILYTLTTNFFKDKLKQLLHKHQRKSIFKIKKKSLSTSIVWIEDSSSLKLGVLNKITLGDSIMKPVS

SEQ ID NO: 19 - DNA, 969 bp, Homo sapiens
atggatccaaccatctcaaccttggacacagaactgacaccaatcaacggaactgaggagactctttgctacaagcagaccttgagcctcacggtgctgacgtgcatcgtttcccttgtcgggctgacaggaaacgcagttgtgctctggctcctgggctgccgcatgcgcaggaacgccttctccatctacatcctcaacttggccgcagcagacttcctcttcctcagcggccgccttatatattccctgttaagcttcatcagtatcccccataccatctctaaaatcctctatcctgtgatgatgttttcctactttgcaggcctgagctttctgagtgccgtgagcaccgagcgctgcctgtccgtcctgtggcccatctggtaccgctgccaccgccccacacacctgtcagcggtggtgtgtgtcctgctctgggccctgtccctgctgcggagcatcctggagtggatgttatgtggcttcctgttcagtggtgctgattctgcttggtgtcaaacatcagatttcatcacagtcgcgtggctgatttttttatgtgtggttctctgtgggtccagcctggtcctgctgatcaggattctctgtggatcccggaagataccgctgaccaggctgtacgtgaccatcctgctcacagtactggtcttcctcctctgtggcctgccctttggcattcagtttttcctatttttatggatccacgtggacagggaagtcttattttgtcatgttcatctagtttctattttcctgtccgctcttaacagcagtgccaaccccatcatttacttcttcgtgggctcctttaggcagcgtcaaaataggcagaacctgaagctggttctccagagggctctgcaggacgcgtctgaggtggatgaaggtggagggcagcttcctgaggaaatcctggagctgtcgggaagcagattggagcagtga

SEQ ID NO: 20 - protein, 322 aa, Homo sapiens
MDPTISTLDTELTPINGTEETLCYKQTLSLTVLTCIVSLVGLTGNAVVLWLLGCRMRRNAFSIYILNLAAADFLFLSGRLIYSLLSFISIPHTISKILYPVMMFSYFAGLSFLSAVSTERCLSVLWPIWYRCHRPTHLSAVVCVLLWALSLLRSILEWMLCGFLFSGADSAWCQTSDFITVAWLIFLCVVLCGSSLVLLIRILCGSRKIPLTRLYVTILLTVLVFLLCGLPFGIQFFLFLWIHVDREVLFCHVHLVSIFLSALNSSANPIIYFFVGSFRQRQNRQNLKLVLQRALQDASEVDEGGGQLPEEILELSGSRLEQ

SEQ ID NO: 21 - DNA, 1305 bp, Homo sapiens
atggaggatctctttagcccctcaattctgccgccggcgcccaacatttccgtgcccatcttgctgggctggggtctcaacctgaccttggggcaaggagcccctgcctctgggccgcccagccgccgcgtccgcctggtgttcctgggggtcatcctggtggtggcggtggcaggcaacaccacagtgctgtgccgcctgtgcggcggcggcgggccctgggcgggccccaagcgtcgcaagatggacttcctgctggtgcagctggccctggcggacctgtacgcgtgcgggggcacggcgctgtcacagctggcctgggaactgctgggcgagccccgcgcggccacgggggacctggcgtgccgcttcctgcagctgctgcaggcatccgggcggggcgcctcggcccacctcgtggtgctcatcgccctcgagcgccggcgcgcggtgcgtcttccgcacggccggccgctgcccgcgcgtgccctcgccgccctgggctggctgctggcactgctgctggcgctgcccccggccttcgtggtgcgcggggactccccctcgccgctgccgccgccgccgccgccaacgtccctgcagccaggcgcgcccccggccgcccgcgcctggccgggggagcgtcgctgccacgggatcttcgcgcccctgccgcgctggcacctgcaggtctacgcgttctacgaggccgtcgcgggcttcgtcgcgcctgttacggtcctgggcgtcgcttgcggccacctactctccgtctggtggcggcaccggccgcaggcccccgcggctgcagcgccctggtcggcgagcccaggtcgagcccctgcgcccagcgcgctgccccgcgccaaggtgcagagcctgaagatgagcctgctgctggcgctgctgttcgtgggctgcgagctgccctactttgccgcccggctggcggccgcgtggtcgtccgggcccgcgggagactgggagggagagggcctgtcggcggcgctgcgcgtggtggcgatggccaacagcgctctcaatcccttcgtctacctcttcttccaggcgggcgactgccggctccggcgacagctgcggaagcggctgggctctctgtgctgcgcgccgcagggaggcgcggaggacgaggaggggccccggggccaccaggcgctctaccgccaacgctggccccaccctcattatcaccatgctcggcgggaaccgctggacgagggcggcttgcgcccaccccctccgcgccccagacccctgccttgctcctgcgaaagtgccttctag

SEQ ID NO: 22 - protein, 434 aa, Homo sapiens
MEDLFSPSILPPAPNISVPILLGWGLNLTLGQGAPASGPPSRRVRLVFLGVILVVAVAGNTTVLCRLCGGGGPWAGPKRRKMDFLLVQLALADLYACGGTALSQLAWELLGEPRAATGDLACRFLQLLQASGRGASAHLVVLIALERRRAVRLPHGRPLPARALAALGWLLALLLALPPAFVVRGDSPSPLPPPPPPTSLQPGAPPAARAWPGERRCHGIFAPLPRWHLQVYAFYEAVAGFVAPVTVLGVACGHLLSVWWRHRPQAPAAAAPWSASPGRAPAPSALPRAKVQSLKMSLLLALLFVGCELPYFAARLAAAWSSGPAGDWEGEGLSAALRVVAMANSALNPFVYLFFQAGDCRLRRQLRKRLGSLCCAPQGGAEDEEGPRGHQALYRQRWPHPHYHHARREPLDEGGLRPPPPRPRPLPCSCESAF

SEQ ID NO: 23 - DNA, 1041 bp, Homo sapiens
atgtacaacgggtcgtgctgccgcatcgagggggacaccatctcccaggtgatgccgccgctgctcattgtggcctttgtgctgggcgcactaggcaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcggacaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccatggggccctgcacataaccctcagcttcacctacatgaacagcatgctggatcccctggtgtattatttttcaagcccctcctttcccaaattctacaacaagctcaaaatctgcagtctgaaacccaagcagccaggacactcaaaaacacaaaggccggaagagatgccaatttcgaacctcggtcgcaggagttgcatcagtgtggcaaatagtttccaaagccagtctgatgggcaatgggatccccacattgttgagtggcactga

SEQ ID NO: 24 - protein, 346 aa, Homo sapiens
MYNGSCCRIEGDTISQVMPPLLIVAFVLGALGNGVALCGFCFHMKTWKPSTVYLFNLAVADFLLMICLPFRTDYYLRRRHWAFGDIPCRVGLFTLAMNRAGSIVFLTVVAADRYFKVVHPHHAVNTISTRVAAGIVCTLWALVILGTVYLLLENHLCVQETAVSCESFIMESANGWHDIMFQLEFFMPLGIILFCSFKIVWSLRRRQQLARQARMKKATRFIMVVAIVFITCYLPSVSARLYFLWTVPSSACDPSVHGALHITLSFTYMNSMLDPLVYYFSSPSFPKFYNKLKICSLKPKQPGHSKTQRPEEMPISNLGRRSCISVANSFQSQSDGQWDPHIVEWH

SEQ ID NO: 25 - DNA, 1011 bp, Homo  sapiens
atgaacaacaatacaacatgtattcaaccatctatgatctcttccatggctttaccaatcatttacatcctcctttgtattgttggtgtttttggaaacactctctctcaatggatatttttaacaaaaataggtaaaaaaacatcaacgcacatctacctgtcacaccttgtgactgcaaacttacttgtgtgcagtgccatgcctttcatgagtatctatttcctgaaaggtttccaatgggaatatcaatctgctcaatgcagagtggtcaattttctgggaactctatccatgcatgcaagtatgtttgtcagtctcttaattttaagttggattgccataagccgctatgctaccttaatgcaaaaggattcctcgcaagagactacttcatgctatgagaaaatattttatggccatttactgaaaaaatttcgccagcccaactttgctagaaaactatgcatttacatatggggagttgtactgggcataatcattccagttaccgtatactactcagtcatagaggctacagaaggagaagagagcctatgctacaatcggcagatggaactaggagccatgatctctcagattgcaggtctcattggaaccacatttattggattttcctttttagtagtactaacatcatactactcttttgtaagccatctgagaaaaataagaacctgtacgtccattatggagaaagatttgacttacagttctgtgaaaagacatcttttggtcatccagattctactaatagtttgcttccttccttatagtatttttaaacccattttttatgttctacaccaaagagataactgtcagcaattgaattatttaatagaaacaaaaaacattctcacctgtcttgcttcggccagaagtagcacagaccccattatatttcttttattagataaaacattcaagaagacactatataatctctttacaaagtctaattcagcacatatgcaatcatatggttga

SEQ ID NO: 26 - protein, 336 aa, Homo sapiens
MNNNTTCIQPSMISSMALPIIYILLCIVGVFGNTLSQWIFLTKIGKKTSTHIYLSHLVTANLLVCSAMPFMSIYFLKGFQWEYQSAQCRVVNFLGTLSMHASMFVSLLILSWIAISRYATLMQKDSSQETTSCYEKIFYGHLLKKFRQPNFARKLCIYIWGVVLGIIIPVTVYYSVIEATEGEESLCYNRQMELGAMISQIAGLIGTTFIGFSFLVVLTSYYSFVSHLRKIRTCTSIMEKDLTYSSVKRHLLVIQILLIVCFLPYSIFKPIFYVLHQRDNCQQLNYLIETKNILTCLASARSSTDPIIFLLLDKTFKKTLYNLFTKSNSAHMQSYG

SEQ ID NO: 27 - DNA, 1014 bp, Homo sapiens
atgaatgagccactagactatttagcaaatgcttctgatttccccgattatgcagctgcttttggaaattgcactgatgaaaacatcccactcaagatgcactacctccctgttatttatggcattatcttcctcgtgggatttccaggcaatgcagtagtgatatccacttacattttcaaaatgagaccttggaagagcagcaccatcattatgctgaacctggcctgcacagatctgctgtatctgaccagcctccccttcctgattcactactatgccagtggcgaaaactggatctttggagatttcatgtgtaagtttatccgcttcagcttccatttcaacctgtatagcagcatcctcttcctcacctgtttcagcatcttccgctactgtgtgatcattcacccaatgagctgcttttccattcacaaaactcgatgtgcagttgtagcctgtgctgtggtgtggatcatttcactggtagctgtcattccgatgaccttcttgatcacatcaaccaacaggaccaacagatcagcctgtctcgacctcaccagttcggatgaactcaatactattaagtggtacaacctgattttgactgcaactactttctgcctccccttggtgatagtgacactttgctataccacgattatccacactctgacccatggactgcaaactgacagctgccttaagcagaaagcacgaaggctaaccattctgctactccttgcattttacgtatgttttttacccttccatatcttgagggtcattcggatcgaatctcgcctgctttcaatcagttgttccattgagaatcagatccatgaagcttacatcgtttctagaccattagctgctctgaacacctttggtaacctgttactatatgtggtggtcagcgacaactttcagcaggctgtctgctcaacagtgagatgcaaagtaagcgggaaccttgagcaagcaaagaaaattagttactcaaacaacccttga

SEQ ID NO: 28 - protein, 337 aa, Homo sapiens
MNEPLDYLANASDFPDYAAAFGNCTDENIPLKMHYLPVIYGIIFLVGFPGNAVVISTYIFKMRPWKSSTIIMLNLACTDLLYLTSLPFLIHYYASGENWIFGDFMCKFIRFSFHFNLYSSILFLTCFSIFRYCVIIHPMSCFSIHKTRCAVVACAVVWIISLVAVIPMTFLITSTNRTNRSACLDLTSSDELNTIKWYNLILTATTFCLPLVIVTLCYTTIIHTLTHGLQTDSCLKQKARRLTILLLLAFYVCFLPFHILRVIRIESRLLSISCSIENQIHEAYIVSRPLAALNTFGNLLLYVVVSDNFQQAVCSTVRCKVSGNLEQAKKISYSNNP

SEQ ID NO: 29 - DNA, 993 bp, Homo sapiens
atggatccaaccaccccggcctggggaacagaaagtacaacagtgaatggaaatgaccaagcccttcttctgctttgtggcaaggagaccctgatcccggtcttcctgatccttttcattgccctggtcgggctggtaggaaacgggtttgtgctctggctcctgggcttccgcatgcgcaggaacgccttctctgtctacgtcctcagcctggccggggccgacttcctcttcctctgcttccagattataaattgcctggtgtacctcagtaacttcttctgttccatctccatcaatttccctagcttcttcaccactgtgatgacctgtgcctaccttgcaggcctgagcatgctgagcaccgtcagcaccgagcgctgcctgtccgtcctgtggcccatctggtatcgctgccgccgccccagacacctgtcagcggtcgtgtgtgtcctgctctgggccctgtccctactgctgagcatcttggaagggaagttctgtggcttcttatttagtgatggtgactctggttggtgtcagacatttgatttcatcactgcagcgtggctgatttttttattcatggttctctgtgggtccagtctggccctgctggtcaggatcctctgtggctccaggggtctgccactgaccaggctgtacctgaccatcctgctcacagtgctggtgttcctcctctgcggcctgccctttggcattcagtggttcctaatattatggatctggaaggattctgatgtcttattttgtcatattcatccagtttcagttgtcctgtcatctcttaacagcagtgccaaccccatcatttacttcttcgtgggctcttttaggaagcagtggcggctgcagcagccgatcctcaagctggctctccagagggctctgcaggacattgctgaggtggatcacagtgaaggatgcttccgtcagggcaccccggagatgtcgagaagcagtctggtgtag

SEQ ID NO: 30 - protein, 330 aa, Homo sapiens
MDPTTPAWGTESTTVNGNDQALLLLCGKETLIPVFLILFIALVGLVGNGFVLWLLGFRMRRNAFSVYVLSLAGADFLFLCFQIINCLVYLSNFFCSISINFPSFFTTVMTCAYLAGLSMLSTVSTERCLSVLWPIWYRCRRPRHLSAVVCVLLWALSLLLSILEGKFCGFLFSDGDSGWCQTFDFITAAWLIFLFMVLCGSSLALLVRILCGSRGLPLTRLYLTILLTVLVFLLCGLPFGIQWFLILWIWKDSDVLFCHIHPVSVVLSSLNSSANPIIYFFVGSFRKQWRLQQPILKLALQRALQDIAEVDHSEGCFRQGTPEMSRSSLV

SEQ ID NO: 31 - DNA, 1092 bp, Homo sapiens
atgggccccggcgaggcgctgctggcgggtctcctggtgatggtactggccgtggcgctgctatccaacgcactggtgctgctttgttgcgcctacagcgctgagctccgcactcgagcctcaggcgtcctcctggtgaatctgtcgctgggccacctgctgctggcggcgctggacatgcccttcacgctgctcggtgtgatgcgcgggcggacaccgtcggcgcccggcgcatgccaagtcattggcttcctggacaccttcctggcgtccaacgcggcgctgagcgtggcggcgctgagcgcagaccagtggctggcagtgggcttcccactgcgctacgccggacgcctgcgaccgcgctatgccggcctgctgctgggctgtgcctggggacagtcgctggccttctcaggcgctgcacttggctgctcgtggcttggctacagcagcgccttcgcgtcctgttcgctgcgcctgccgcccgagcctgagcgtccgcgcttcgcagccttcaccgccacgctccatgccgtgggcttcgtgctgccgctggcggtgctctgcctcacctcgctccaggtgcaccgggtggcacgcagccactgccagcgcatggacaccgtcaccatgaaggcgctcgcgctgctcgccgacctgcaccccagtgtgcggcagcgctgcctcatccagcagaagcggcgccgccaccgcgccaccaggaagattggcattgctattgcgaccttcctcatctgctttgccccgtatgtcatgaccaggctggcggagctcgtgcccttcgtcaccgtgaacgcccagtggggcatcctcagcaagtgcctgacctacagcaaggcggtggccgacccgttcacgtactctctgctccgccggccgttccgccaagtcctggccggcatggtgcaccggctgctgaagagaaccccgcgcccagcatccacccatgacagctctctggatgtggccggcatggtgcaccagctgctgaagagaaccccgcgcccagcgtccacccacaacggctctgtggacacagagaatgattcctgcctgcagcagacacactga

SEQ ID NO: 32 - protein, 363 aa, Homo sapiens
MGPGEALLAGLLVMVLAVALLSNALVLLCCAYSAELRTRASGVLLVNLSLGHLLLAALDMPFTLLGVMRGRTPSAPGACQVIGFLDTFLASNAALSVAALSADQWLAVGFPLRYAGRLRPRYAGLLLGCAWGQSLAFSGAALGCSWLGYSSAFASCSLRLPPEPERPRFAAFTATLHAVGFVLPLAVLCLTSLQVHRVARSHCQRMDTVTMKALALLADLHPSVRQRCLIQQKRRRHRATRKIGIAIATFLICFAPYVMTRLAELVPFVTVNAQWGILSKCLTYSKAVADPFTYSLLRRPFRQVLAGMVHRLLKRTPRPASTHDSSLDVAGMVHQLLKRTPRPASTHNGSVDTENDSCLQQTH

SEQ ID NO: 33 - DNA, 1125 bp, Homo sapiens
atgcccacactcaatacttctgcctctccacccacattcttctgggccaatgcctccggaggcagtgtgctgagtgctgatgatgctccgatgcctgtcaaattcctagccctgaggctcatggttgccctggcctatgggcttgtgggggccattggcttgctgggaaatttggcggtgctgtgggtactgagtaactgtgcccggagagcccctggcccaccttcagacaccttcgtcttcaacctggctctggcggacctgggactggcactcactctccccttttgggcagccgagtcggcactggactttcactggcccttcggaggtgccctctgcaagatggttctgacggccactgtcctcaacgtctatgccagcatcttcctcatcacagcgctgagcgttgctcgctactgggtggtggccatggctgcggggccaggcacccacctctcactcttctgggcccgaatagccaccctggcagtgtgggcggcggctgccctggtgacggtgcccacagctgtcttcggggtggagggtgaggtgtgtggtgtgcgcctttgcctgctgcgtttccccagcaggtactggctgggggcctaccagctgcagagggtggtgctggctttcatggtgcccttgggcgtcatcaccaccagctacctgctgctgctggccttcctgcagcggcggcaacggcggcggcaggacagcagggtcgtggcccgctctgtccgcatcctggtggcttccttcttcctctgctggtttcccaaccatgtggtcactctctggggtgtcctggtgaagtttgacctggtgccctggaacagtactttctatactatccagacgtatgtcttccctgtcactacttgcttggcacacagcaatagctgcctcaaccctgtgctgtactgtctcctgaggcgggagccccggcaggctctggcaggcaccttcagggatctgcggtcgaggctgtggccccagggcggaggctgggtgcaacaggtggccctaaagcaggtaggcaggcggtgggtcgcaagcaacccccgggagagccgcccttctaccctgctcaccaacctggacagagggacacccgggtga

SEQ ID NO: 34 - protein, 374 aa, Homo sapiens
MPTLNTSASPPTFFWANASGGSVLSADDAPMPVKFLALRLMVALAYGLVGAIGLLGNLAVLWVLSNCARRAPGPPSDTFVFNLALADLGLALTLPFWAAESALDFHWPFGGALCKMVLTATVLNVYASIFLITALSVARYWVVAMAAGPGTHLSLFWARIATLAVWAAAALVTVPTAVFGVEGEVCGVRLCLLRFPSRYWLGAYQLQRVVLAFMVPLGVITTSYLLLLAFLQRRQRRRQDSRVVARSVRILVASFFLCWFPNHVVTLWGVLVKFDLVPWNSTFYTIQTYVFPVTTCLAHSNSCLNPVLYCLLRREPRQALAGTFRDLRSRLWPQGGGWVQQVALKQVGRRWVASNPRESRPSTLLTNLDRGTPG

SEQ ID NO: 35 - DNA, 1092 bp, Homo sapiens
atgaatcggcaccatctgcaggatcactttctggaaatagacaagaagaactgctgtgtgttccgagatgacttcattgtcaaggtgttgccgccggtgttggggctggagtttatcttcgggcttctgggcaatggccttgccctgtggattttctgtttccacctcaagtcctggaaatccagccggattttcctgttcaacctggcagtggctgactttctactgatcatctgcctgcccttcctgatggacaactatgtgaggcgttgggactggaagtttggggacatcccttgccggctgatgctcttcatgttggctatgaaccgccagggcagcatcatcttcctcacggtggtggcggtagacaggtatttccgggtggtccatccccaccacgccctgaacaagatctccaatcggacagcagccatcatctcttgccttctgtggggcatcactattggcctgacagtccacctcctgaagaagaagatgccgatccagaatggcggtgcaaatttgtgcagcagcttcagcatctgccataccttccagtggcacgaagccatgttcctcctggagttcttcctgcccctgggcatcatcctgttctgctcagccagaattatctggagcctgcggcagagacaaatggaccggcatgccaagatcaagagagccatcaccttcatcatggtggtggccatcgtctttgtcatctgcttccttcccagcgtggttgtgcggatccgcatcttctggctcctgcacacttcgggcacgcagaattgtgaagtgtaccgctcggtggacctggcgttctttatcactctcagcttcacctacatgaacagcatgctggaccccgtggtgtactacttctccagcccatcctttcccaacttcttctccactttgatcaaccgctgcctccagaggaagatgacaggtgagccagataataaccgcagcacgagcgtcgagctcacaggggaccccaacaaaaccagaggcgctccagaggcgttaatggccaactccggtgagccatggagcccctcttatctgggcccaacctctccttaa

SEQ ID NO: 36 - protein, 363 aa, Homo sapiens [sequence omitted]

SEQ ID NO: 37 - DNA, 1044 bp, Homo sapiens
atgggggatgagctggcaccttgccctgtgggcactacagcttggccggccctgatccagctcatcagcaagacaccctgcatgccccaagcagccagcaacacttccttgggcctgggggacctcagggtgcccagctccatgctgtactggcttttccttccctcaagcctgctggctgcagccacactggctgtcagccccctgctgctggtgaccatcctgcggaaccaacggctgcgacaggagccccactacctgctcccggctaacatcctgctctcagacctggcctacattctcctccacatgctcatctcctccagcagcctgggtggctgggagctgggccgcatggcctgtggcattctcactgatgctgtcttcgccgcctgcaccagcaccatcctgtccttcaccgccattgtgctgcacacctacctggcagtcatccatccactgcgctacctctccttcatgtcccatggggctgcctggaaggcagtggccctcatctggctggtggcctgctgcttccccacattccttatttggctcagcaagtggcaggatgcccagctggaggagcaaggagcttcatacatcctaccaccaagcatgggcacccagccgggatgtggcctcctggtcattgttacctacacctccattctgtgcgttctgttcctctgcacagctctcattgccaactgtttctggaggatctatgcagaggccaagacttcaggcatctgggggcagggctattcccgggccaggggcaccctgctgatccactcagtgctgatcacattgtacgtgagcacaggggtggtgttctccctggacatggtgctgaccaggtaccaccacattgactctgggactcacacatggctcctggcagctaacagtgaggtactcatgatgcttccccgtgccatgctcccatacctgtacctgctccgctaccggcagctgttgggcatggtccggggccacctcccatccaggaggcaccaggccatctttaccatttcctag

SEQ ID NO: 38 - protein, 347 aa, Homo sapiens
MGDELAPCPVGTTAWPALIQLISKTPCMPQAASNTSLGLGDLRVPSSMLYWLFLPSSLLAAATLAVSPLLLVTILRNQRLRQEPHYLLPANILLSDLAYILLHMLISSSSLGGWELGRMACGILTDAVFAACTSTILSFTAIVLHTYLAVIHPLRYLSFMSHGAAWKAVALIWLVACCFPTFLIWLSKWQDAQLEEQGASYILPPSMGTQPGCGLLVIVTYTSILCVLFLCTALIANCFWRIYAEAKTSGIWGQGYSRARGTLLIHSVLITLYVSTGVVFSLDMVLTRYHHIDSGTHTWLLAANSEVLMMLPRAMLPYLYLLRYRQLLGMVRGHLPSRRHQAIFTIS

SEQ ID NO: 39 - DNA, 1023 bp, Homo sapiens
atgaatccatttcatgcatcttgttggaacacctctgccgaacttttaaacaaatcctggaataaagagtttgcttatcaaactgccagtgtggtagatacagtcatcctcccttccatgattgggattatctgttcaacagggctggttggcaacatcctcattgtattcactataataagatccaggaaaaaaacagtccctgacatctatatctgcaacctggctgtggctgatttggtccacatagttggaatgccttttcttattcaccaatgggcccgagggggagagtgggtgtttggggggcctctctgcaccatcatcacatccctggatacttgtaaccaatttgcctgtagtgccatcatgactgtaatgagtgtggacaggtactttgccctcgtccaaccatttcgactgacacgttggagaacaaggtacaagaccatccggatcaatttgggcctttgggcagcttcctttatcctggcattgcctgtctgggtctactcgaaggtcatcaaatttaaagacggtgttgagagttgtgcttttgatttgacatcccctgacgatgtactctggtatacactttatttgacgataacaactttttttttccctctacccttgattttggtgtgctatattttaattttatgctatacttgggagatgtatcaacagaataaggatgccagatgctgcaatcccagtgtaccaaaacagagagtgatgaagttgacaaagatggtgctggtgctggtggtagtctttatcctgagtgctgccccttatcatgtgatacaactggtgaacttacagatggaacagcccacactggccttctatgtgggttattacctctccatctgtctcagctatgccagcagcagcattaacccttttctctacatcctgctgagtggaaatttccagaaacgtctgcctcaaatccaaagaagagcgactgagaaggaaatcaacaatatgggaaacactctgaaatcacacttttag

SEQ ID NO: 40 - protein, 340 aa, Homo sapiens
MNPFHASCWNTSAELLNKSWNKEFAYQTASVVDTVILPSMIGIICSTGLVGNILIVFTIIRSRKKTVPDIYICNLAVADLVHIVGMPFLIHQWARGGEWVFGGPLCTIITSLDTCNQFACSAIMTVMSVDRYFALVQPFRLTRWRTRYKTIRINLGLWAASFILALPVWVYSKVIKFKDGVESCAFDLTSPDDVLWYTLYLTITTFFFPLPLILVCYILILCYTWEMYQQNKDARCCNPSVPKQRVMKLTKMVLVLVVVFILSAAPYHVIQLVNLQMEQPTLAFYVGYYLSICLSYASSSINPFLYILLSGNFQKRLPQIQRRATEKEINNMGNTLKSHF

SEQ ID NO: 41 - DNA, 24 bp, Artificial Sequence
  misc_feature: Novel Sequence
cttgcagacatcaccatggcagcc

SEQ ID NO: 42 - DNA, 24 bp, Artificial Sequence
  misc_feature: Novel Sequence
gtgatgctctgagtactggactgg

SEQ ID NO: 43 - DNA, 20 bp, Artificial Sequence
  misc_feature: Novel Sequence
gaagctgtgaagagtgatgc

SEQ ID NO: 44 - DNA, 24 bp, Artificial Sequence
  misc_feature: Novel Sequence
gtcagcaatattgataagcagcag

SEQ ID NO: 45 - DNA, 27 bp, Artificial Sequence
  misc_feature: Novel Sequence
ccatggggaacgattctgtcagctacg

SEQ ID NO: 46 - DNA, 24 bp, Artificial Sequence
  misc_feature: Novel Sequence
gctatgcctgaagccagtcttgtg

SEQ ID NO: 47 - DNA, 26 bp, Artificial Sequence
  misc_feature: Novel Sequence
ccaggatgttgtgtcaccgtggtggc

SEQ ID NO: 48 - DNA, 26 bp, Artificial Sequence
  misc_feature: Novel Sequence
cacagcgctgcagccctgcagctggc

SEQ ID NO: 49 - DNA, 26 bp, Artificial Sequence
  misc_feature: Novel Sequence
cttcctctcgtagggatgaaccagac

SEQ ID NO: 50 - DNA, 26 bp, Artificial Sequence
  misc_feature: Novel Sequence
ctcgcacaggtgggaagcacctgtgg

SEQ ID NO: 51 - DNA, 23 bp, Artificial Sequence
  misc_feature: Novel Sequence
gcctgtgacaggaggtaccctgg

SEQ ID NO: 52 - DNA, 25 bp, Artificial Sequence
  misc_feature: Novel Sequence
catatccctccgagtgtccagcggc

SEQ ID NO: 53 - DNA, 31 bp, Artificial Sequence
  misc_feature: Novel Sequence
gcatggagagaaaatttatgtccttgcaacc

SEQ ID NO: 54 - DNA, 27 bp, Artificial Sequence
  misc_feature: Novel Sequence
caagaacaggtctcatctaagagctcc

SEQ ID NO: 55 - DNA, 26 bp, Artificial Sequence
  misc_feature: Novel Sequence
gctgttgccatgacgtccacctgcac

SEQ ID NO: 56 - DNA, 26 bp, Artificial Sequence
  misc_feature: Novel Sequence
ggacagttcaaggtttgccttagaac

SEQ ID NO: 57 - DNA, 23 bp, Artificial Sequence
  misc_feature: Novel Sequence
ctttcgatactgctcctatgctc

SEQ ID NO: 58 - DNA, 26 bp, Artificial Sequence
  misc_feature: Novel Sequence
gtagtccactgaaagtccagtgatcc

SEQ ID NO: 59 - DNA, 26 bp, Artificial Sequence
  misc_feature: Novel Sequence
tttctgagcatggatccaaccatctc

SEQ ID NO: 60 - DNA, 25 bp, Artificial Sequence
  misc_feature: Novel Sequence
ctgtctgacagggcagaggctcttc

SEQ ID NO: 61 - DNA, 28 bp, Artificial Sequence
  misc_feature: Novel Sequence
ggaactcgtatagacccagcgtcgctcc

SEQ ID NO: 62 - DNA, 28 bp, Artificial Sequence
  misc_feature: Novel Sequence
ggaggttgcgccttagcgacagatgacc

SEQ ID NO: 63 - DNA, 22 bp, Artificial Sequence
  misc_feature: Novel Sequence
ctgcacccggacacttgctctg

SEQ ID NO: 64 - DNA, 25 bp, Artificial Sequence
  misc_feature: Novel Sequence
gtctgcttgttcagtgccactcaac

SEQ ID NO: 65 - DNA, 26 bp, Artificial Sequence
  misc_feature: Novel Sequence
tatctgcaattctattctagctcctg

SEQ ID NO: 66 - DNA, 26 bp, Artificial Sequence
  misc_feature: Novel Sequence
tgtccctaataaagtcacatgaatgc

SEQ ID NO: 67 - DNA, 23 bp, Artificial Sequence
  misc_feature: Novel Sequence
ggagacaaccatgaatgagccac

SEQ ID NO: 68 - DNA, 24 bp, Artificial Sequence
  misc_feature: Novel Sequence
tatttcaagggttgtttgagtaac

SEQ ID NO: 69 - DNA, 27 bp, Artificial Sequence
  misc_feature: Novel Sequence
ggcaccagtggaggttttctgagcatg

SEQ ID NO: 70 - DNA, 27 bp, Artificial Sequence
  misc_feature: Novel Sequence
ctgatggaagtagaggctgtccatctc

SEQ ID NO: 71 - DNA, 23 bp, Artificial Sequence
  misc_feature: Novel Sequence
cctggcgagccgctagcgccatg

SEQ ID NO: 72 - DNA, 23 bp, Artificial Sequence
  misc_feature: Novel Sequence
atgagccctgccaggccctcagt

SEQ ID NO: 73 - DNA, 27 bp, Artificial Sequence
  misc_feature: Novel Sequence
ctgcgatgcccacactcaatacttctg

SEQ ID NO: 74 - DNA, 27 bp, Artificial Sequence
  misc_feature: Novel Sequence
aaggatcctacacttggtggatctcag

SEQ ID NO: 75 - DNA, 22 bp, Artificial Sequence
  misc_feature: Novel Sequence
gctggagcattcactaggcgag

SEQ ID NO: 76 - DNA, 24 bp, Artificial Sequence
  misc_feature: Novel Sequence
agatcctggttcttggtgacaatg

SEQ ID NO: 77 - DNA, 24 bp, Artificial Sequence
  misc_feature: Novel Sequence
agccatccctgccaggaagcatgg

SEQ ID NO: 78 - DNA, 27 bp, Artificial Sequence
  misc_feature: Novel Sequence
ccagactgtggactcaagaactctagg

SEQ ID NO: 79 - DNA, 28 bp, Artificial Sequence
  misc_feature: Novel Sequence
agtccacgaacaatgaatccatttcatg

SEQ ID NO: 80 - DNA, 25 bp, Artificial Sequence
  misc_feature: Novel Sequence
atcatgtctagactcatggtgatcc

SEQ ID NO: 81 - DNA, 30 bp, Artificial Sequence
  misc_feature: Novel Sequence
ggggagggaaagcaaaggtggtcctcctgg

SEQ ID NO: 82 - DNA, 30 bp, Artificial Sequence
  misc_feature: Novel Sequence
ccaggagaaccacctttgctttccctcccc

SEQ ID NO: 83 - DNA, 1356 bp, Homo sapiens
atggagtcctcacccatcccccagtcatcagggaactcttccactttggggagggtccctcaaaccccaggtccctctactgccagtggggtcccggaggtggggctacgggatgttgcttcggaatctgtggccctcttcttcatgctcctgctggacttgactgctgtggctggcaatgccgctgtgatggccgtgatcgccaagacgcctgccctccgaaaatttgtcttcgtcttccacctctgcctggtggacctgctggctgccctgaccctcatgcccctggccatgctctccagctctgccctctttgaccacgccctctttggggaggtggcctgccgcctctacttgtttctgagcgtgtgctttgtcagcctggccatcctctcggtgtcagccatcaatgtggagcgctactattacgtagtccaccccatgcgctacgaggtgcgcatgacgctggggctggtggcctctgtgctggtgggtgtgtgggtgaaggccttggccatggcttctgtgccagtgttgggaagggtctcctgggaggaaggagctcccagtgtccccccaggctgttcactccagtggagccacagtgcctactgccagctttttgtggtggtctttgctgtcctttactttctgttgcccctgctcctcatacttgtggtctactgcagcatgttccgagtggcccgcgtggctgccatgcagcacgggccgctgcccacgtggatggagacaccccggcaacgctccgaatctctcagcagccgctccacgatggtcaccagctcgggggccccccagaccaccccacaccggacgtttgggggagggaaagcaaaggtggttctcctggctgtggggggacagttcctgctctgttggttgccctacttctctttccacctctatgttgccctgagtgctcagcccatttcaactgggcaggtggagagtgtggtcacctggattggctacttttgcttcacttccaaccctttcttctatggatgtctcaaccggcagatccggggggagctcagcaagcagtttgtctgcttcttcaagccagctccagaggaggagctgaggctgcctagccgggagggctccattgaggagaacttcctgcagttccttcaggggactggctgtccttctgagtcctgggtttcccgacccctacccagccccaagcaggagccacctgctgttgactttcgaatcccaggccagatagctgaggagacctctgagttcctggagcagcaactcaccagcgacatcatcatgtcagacagctacctccgtcctgccgcctcaccccggctggagtcatga

SEQ ID NO: 84 - protein, 451 aa, Homo sapiens
MESSPIPQSSGNSSTLGRVPQTPGPSTASGVPEVGLRDVASESVALFFMLLLDLTAVAGNAAVMAVIAKTPALRKFVFVFHLCLVDLLAALTLMPLAMLSSSALFDHALFGEVACRLYLFLSVCFVSLAILSVSAINVERYYYVVHPMRYEVRMTLGLVASVLVGVWVKALAMASVPVLGRVSWEEGAPSVPPGCSLQWSHSAYCQLFVVVFAVLYFLLPLLLILVVYCSMFRVARVAAMQHGPLPTWMETPRQRSESLSSRSTMVTSSGAPQTTPHRTFGGGKAKVVLLAVGGQFLLCWLPYFSFHLYVALSAQPISTGQVESVVTWIGYFCFTSNPFFYGCLNRQIRGELSKQFVCFFKPAPEEELRLPSREGSIEENFLQFLQGTGCPSESWVSRPLPSPKQEPPAVDFRIPGQIAEETSEFLEQQLTSDIIMSDSYLRPAASPRLES

SEQ ID NO: 85 - DNA, 28 bp, Homo sapiens
caggaaggcaaagaccaccatcatcatc

SEQ ID NO: 86 - DNA, 28 bp, Homo sapiens
gatgatgatggtggtctttgccttcctg

SEQ ID NO: 87 - DNA, 1041 bp, Homo sapiens
atggagagaaaatttatgtccttgcaaccatccatctccgtatcagaaatggaaccaaatggcaccttcagcaataacaacagcaggaactgcacaattgaaaacttcaagagagaatttttcccaattgtatatctgataatatttttctggggagtcttgggaaatgggttgtccatatatgttttcctgcagccttataagaagtccacatctgtgaacgttttcatgctaaatctggccatttcagatctcctgttcataagcacgcttcccttcagggctgactattatcttagaggctccaattggatatttggagacctggcctgcaggattatgtcttattccttgtatgtcaacatgtacagcagtatttatttcctgaccgtgctgagtgttgtgcgtttcctggcaatggttcacccctttcggcttctgcatgtcaccagcatcaggagtgcctggatcctctgtgggatcatatggatccttatcatggcttcctcaataatgctcctggacagtggctctgagcagaacggcagtgtcacatcatgcttagagctgaatctctataaaattgctaagctgcagaccatgaactatattgccttggtggtgggctgcctgctgccatttttcacactcagcatctgttatctgctgatcattcgggttctgttaaaagtggaggtcccagaatcggggctgcgggtttctcacaggaaggcaaagaccaccatcatcatcaccttgatcatcttcttcttgtgtttcctgccctatcacacactgaggaccgtccacttgacgacatggaaagtgggtttatgcaaagacagactgcataaagctttggttatcacactggccttggcagcagccaatgcctgcttcaatcctctgctctattactttgctggggagaattttaaggacagactaaagtctgcactcagaaaaggccatccacagaaggcaaagacaaagtgtgttttccctgttagtgtgtggttgagaaaggaaacaagagtataa

SEQ ID NO: 88 - protein, 346 aa, Homo sapiens
MERKFMSLQPSISVSEMEPNGTFSNNNSRNCTIENFKREFFPIVYLIIFFWGVLGNGLSIYVFLQPYKKSTSVNVFMLNLAISDLLFISTLPFRADYYLRGSNWIFGDLACRIMSYSLYVNMYSSIYFLTVLSVVRFLAMVHPFRLLHVTSIRSAWILCGIIWILIMASSIMLLDSGSEQNGSVTSCLELNLYKIAKLQTMNYIALVVGCLLPFFTLSICYLLIIRVLLKVEVPESGLRVSHRKAKTTIIITLIIFFLCFLPYHTLRTVHLTTWKVGLCKDRLHKALVITLALAAANACFNPLLYYFAGENFKDRLKSALRKGHPQKAKTKCVFPVSVWLRKETRV

SEQ ID NO: 89 - DNA, 28 bp, Artificial Sequence
  misc_feature: Novel Sequence
ccagtgcaaagctaagaaagtgatcttc

SEQ ID NO: 90 - DNA, 28 bp, Artificial Sequence
  misc_feature: Novel Sequence
gaagatcactttcttagctttgcactgg

SEQ ID NO: 91 - DNA, 1527 bp, Homo sapiens
atgacgtccacctgcaccaacagcacgcgcgagagtaacagcagccacacgtgcatgcccctctccaaaatgcccatcagcctggcccacggcatcatccgctcaaccgtgctggttatcttcctcgccgcctctttcgtcggcaacatagtgctggcgctagtgttgcagcgcaagccgcagctgctgcaggtgaccaaccgttttatctttaacctcctcgtcaccgacctgctgcagatttcgctcgtggccccctgggtggtggccacctctgtgcctctcttctggcccctcaacagccacttctgcacggccctggttagcctcacccacctgttcgccttcgccagcgtcaacaccattgtcgtggtgtcagtggatcgctacttgtccatcatccaccctctctcctacccgtccaagatgacccagcgccgcggttacctgctcctctatggcacctggattgtggccatcctgcagagcactcctccactctacggctggggccaggctgcctttgatgagcgcaatgctctctgctccatgatctggggggccagccccagctacactattctcagcgtggtgtccttcatcgtcattccactgattgtcatgattgcctgctactccgtggtgttctgtgcagcccggaggcagcatgctctgctgtacaatgtcaagagacacagcttggaagtgcgagtcaaggactgtgtggagaatgaggatgaagagggagcagagaagaaggaggagttccaggatgagagtgagtttcgccgccagcatgaaggtgaggtcaaggccaaggagggcagaatggaagccaaggacggcagcctgaaggccaaggaaggaagcacggggaccagtgagagtagtgtagaggccaggggcagcgaggaggtcagagagagcagcacggtggccagcgacggcagcatggagggtaaggaaggcagcaccaaagttgaggagaacagcatgaaggcagacaagggtcgcacagaggtcaaccagtgcagcattgacttgggtgaagatgacatggagtttggtgaagacgacatcaatttcagtgaggatgacgtcgaggcagtgaacatcccggagagcctcccacccagtcgtcgtaacagcaacagcaaccctcctctgcccaggtgctaccagtgcaaagctaagaaagtgatcttcatcatcattttctcctatgtgctatccctggggccctactgctttttagcagtcctggccgtgtgggtggatgtcgaaacccaggtaccccagtgggtgatcaccataatcatctggcttttcttcctgcagtgctgcatccacccctatgtctatggctacatgcacaagaccattaagaaggaaatccaggacatgctgaagaagttcttctgcaaggaaaagcccccgaaagaagatagccacccagacctgcccggaacagagggtgggactgaaggcaagattgtcccttcctacgattctgctacttttccttga

SEQ ID NO: 92 - protein, 508 aa, Homo sapiens
MTSTCTNSTRESNSSHTCMPLSKMPISLAHGIIRSTVLVIFLAASFVGNIVLALVLQRKPQLLQVTNRFIFNLLVTDLLQISLVAPWVVATSVPLFWPLNSHFCTALVSLTHLFAFASVNTIVVVSVDRYLSIIHPLSYPSKMTQRRGYLLLYGTWIVAILQSTPPLYGWGQAAFDERNALCSMIWGASPSYTILSVVSFIVIPLIVMIACYSVVFCAARRQHALLYNVKRHSLEVRVKDCVENEDEEGAEKKEEFQDESEFRRQHEGEVKAKEGRMEAKDGSLKAKEGSTGTSESSVEARGSEEVRESSTVASDGSMEGKEGSTKVEENSMKADKGRTEVNQCSIDLGEDDMEFGEDDINFSEDDVEAVNIPESLPPSRRNSNSNPPLPRCYQCKAKKVIFIIIFSYVLSLGPYCFLAVLAVWVDVETQVPQWVITIIIWLFFLQCCIHPYVYGYMHKTIKKEIQDMLKKFFCKEKPPKEDSHPDLPGTEGGTEGKIVPSYDSATFP

SEQ ID NO: 93 - DNA, 29 bp, Artificial Sequence
  misc_feature: Novel Sequence
gccgccaccgcgccaagaggaagattggc

SEQ ID NO: 94 - DNA, 29 bp, Artificial Sequence
  misc_feature: Novel Sequence
gccaatcttcctcttggcgcggtggcggc

SEQ ID NO: 95 - DNA, 1092 bp, Homo sapiens
atgggccccggcgaggcgctgctggcgggtctcctggtgatggtactggccgtggcgctgctatccaacgcactggtgctgctttgttgcgcctacagcgctgagctccgcactcgagcctcaggcgtcctcctggtgaatctgtcgctgggccacctgctgctggcggcgctggacatgcccttcacgctgctcggtgtgatgcgcgggcggacaccgtcggcgcccggcgcatgccaagtcattggcttcctggacaccttcctggcgtccaacgcggcgctgagcgtggcggcgctgagcgcagaccagtggctggcagtgggcttcccactgcgctacgccggacgcctgcgaccgcgctatgccggcctgctgctgggctgtgcctggggacagtcgctggccttctcaggcgctgcacttggctgctcgtggcttggctacagcagcgccttcgcgtcctgttcgctgcgcctgccgcccgagcctgagcgtccgcgcttcgcagccttcaccgccacgctccatgccgtgggcttcgtgctgccgctggcggtgctctgcctcacctcgctccaggtgcaccgggtggcacgcagccactgccagcgcatggacaccgtcaccatgaaggcgctcgcgctgctcgccgacctgcaccccagtgtgcggcagcgctgcctcatccagcagaagcggcgccgccaccgcgccaccaggaagattggcattgctattgcgaccttcctcatctgctttgccccgtatgtcatgaccaggctggcggagctcgtgcccttcgtcaccgtgaacgcccagaagggcatcctcagcaagtgcctgacctacagcaaggcggtggccgacccgttcacgtactctctgctccgccggccgttccgccaagtcctggccggcatggtgcaccggctgctgaagagaaccccgcgcccagcatccacccatgacagctctctggatgtggccggcatggtgcaccagctgctgaagagaaccccgcgcccagcgtccacccacaacggctctgtggacacagagaatgattcctgcctgcagcagacacactga

SEQ ID NO: 96 - protein, 363 aa, Homo sapiens
MGPGEALLAGLLVMVLAVALLSNALVLLCCAYSAELRTRASGVLLVNLSLGHLLLAALDMPFTLLGVMRGRTPSAPGACQVIGFLDTFLASNAALSVAALSADQWLAVGFPLRYAGRLRPRYAGLLLGCAWGQSLAFSGAALGCSWLGYSSAFASCSLRLPPEPERPRFAAFTATLHAVGFVLPLAVLCLTSLQVHRVARSHCQRMDTVTMKALALLADLHPSVRQRCLIQQKRRRHRATRKIGIAIATFLICFAPYVMTRLAELVPFVTVNAQKGILSKCLTYSKAVADPFTYSLLRRPFRQVLAGMVHRLLKRTPRPASTHDSSLDVAGMVHQLLKRTPRPASTHNGSVDTENDSCLQQTH

SEQ ID NO: 97 - DNA, 34 bp, Artificial Sequence
  misc_feature: Novel Sequence
gatctctagaatggagtcctcacccatcccccag

SEQ ID NO: 98 - DNA, 36 bp, Artificial Sequence
  misc_feature: Novel Sequence
gatcgatatccgtgactccagccggggtgaggcggc

SEQ ID NO: 99 - DNA, 2610 bp, Homo sapiens and Rat
atggagtcctcacccatcccccagtcatcagggaactcttccactttggggagggtccctcaaaccccaggtccctctactgccagtggggtcccggaggtggggctacgggatgttgcttcggaatctgtggccctcttcttcatgctcctgctggacttgactgctgtggctggcaatgccgctgtgatggccgtgatcgccaagacgcctgccctccgaaaatttgtcttcgtcttccacctctgcctggtggacctgctggctgccctgaccctcatgcccctggccatgctctccagctctgccctctttgaccacgccctctttggggaggtggcctgccgcctctacttgtttctgagcgtgtgctttgtcagcctggccatcctctcggtgtcagccatcaatgtggagcgctactattacgtagtccaccccatgcgctacgaggtgcgcatgacgctggggctggtggcctctgtgctggtgggtgtgtgggtgaaggccttggccatggcttctgtgccagtgttgggaagggtctcctgggaggaaggagctcccagtgtccccccaggctgttcactccagtggagccacagtgcctactgccagctttttgtggtggtctttgctgtcctttactttctgttgcccctgctcctcatacttgtggtctactgcagcatgttccgagtggcccgcgtggctgccatgcagcacgggccgctgcccacgtggatggagacaccccggcaacgctccgaatctctcagcagccgctccacgatggtcaccagctcgggggccccccagaccaccccacaccggacgtttgggggagggaaagcagcagtggttctcctggctgtggggggacagttcctgctctgttggttgccctacttctctttccacctctatgttgccctgagtgctcagcccatttcaactgggcaggtggagagtgtggtcacctggattggctacttttgcttcacttccaaccctttcttctatggatgtctcaaccggcagatccggggggagctcagcaagcagtttgtctgcttcttcaagccagctccagaggaggagctgaggctgcctagccgggagggctccattgaggagaacttcctgcagttccttcaggggactggctgtccttctgagtcctgggtttcccgacccctacccagccccaagcaggagccacctgctgttgactttcgaatcccaggccagatagctgaggagacctctgagttcctggagcagcaactcaccagcgacatcatcatgtcagacagctacctccgtcctgccgcctcaccccggctggagtcagcgatatctgcagaattccaccacactggactagtggatccgagctcggtaccaagcttgggctgcaggtcgatgggctgcctcggcaacagtaagaccgaggaccagcgcaacgaggagaaggcgcagcgcgaggccaacaaaaagatcgagaagcagctgcagaaggacaagcaggtctaccgggccacgcaccgcctgctgctgctgggtgctggagagtctggcaaaagcaccattgtgaagcagatgaggatcctacatgttaatgggtttaacggagagggcggcgaagaggacccgcaggctgcaaggagcaacagcgatggtgagaaggccaccaaagtgcaggacatcaaaaacaacctgaaggaggccattgaaaccattgtggccgccatgagcaacctggtgccccccgtggagctggccaaccctgagaaccagttcagagtggactacattctgagcgtgatgaacgtgccaaactttgacttcccacctgaattctatgagcatgccaaggctctgtgggaggatgagggagttcgtgcctgctacgagcgctccaacgagtaccagctgatcgactgtgcccagtacttcctggacaagattgatgtgatcaagcaggccgactacgtgccaagtgaccaggacctgcttcgctgccgcgtcctgacctctggaatctttgagaccaagttccaggtggacaaagtcaacttccacatgttcgatgtgggcggccagcgcgatgaacgccgcaagtggatccagtgcttcaatgatgtgactgccatcatcttcgtggtggccagcagcagctacaacatggtcatccgggaggacaaccagaccaaccgtctgcaggaggctctgaacctcttcaagagcatctggaacaacagatggctgcgtaccatctctgtgatcctcttcctcaacaagcaagatctgcttgctgagaaggtcctcgctgggaaatcgaagattgaggactactttccagagttcgctcgctacaccactcctgaggatgcgactcccgagcccggagaggacccacgcgtgacccgggccaagtacttcatccgggatgagtttctgagaatcagcactgctagtggagatggacgtcactactgctaccctcactttacctgcgccgtggacactgagaacatccgccgtgtcttcaacgactgccgtgacatcatccagcgcatgcatcttcgccaatacgagctgctctaa

SEQ ID NO: 100 - protein, 869 aa, Homo sapiens and Rat [sequence omitted]

SEQ ID NO: 101 - DNA, 30 bp, Artificial Sequence
  misc_feature: Novel Sequence
tctagaatgacgtccacctgcaccaacagc

SEQ ID NO: 102 - DNA, 34 bp, Artificial Sequence
  misc_feature: Novel Sequence
gatatcgcaggaaaagtagcagaatcgtaggaag

SEQ ID NO: 103 - DNA, 2781 bp, Homo Sapiens and Rat
atgacgtccacctgcaccaacagcacgcgcgagagtaacagcagccacacgtgcatgcccctctccaaaatgcccatcagcctggcccacggcatcatccgctcaaccgtgctggttatcttcctcgccgcctctttcgtcggcaacatagtgctggcgctagtgttgcagcgcaagccgcagctgctgcaggtgaccaaccgttttatctttaacctcctcgtcaccgacctgctgcagatttcgctcgtggccccctgggtggtggccacctctgtgcctctcttctggcccctcaacagccacttctgcacggccctggttagcctcacccacctgttcgccttcgccagcgtcaacaccattgtcgtggtgtcagtggatcgctacttgtccatcatccaccctctctcctacccgtccaagatgacccagcgccgcggttacctgctcctctatggcacctggattgtggccatcctgcagagcactcctccactctacggctggggccaggctgcctttgatgagcgcaatgctctctgctccatgatctggggggccagccccagctacactattctcagcgtggtgtccttcatcgtcattccactgattgtcatgattgcctgctactccgtggtgttctgtgcagcccggaggcagcatgctctgctgtacaatgtcaagagacacagcttggaagtgcgagtcaaggactgtgtggagaatgaggatgaagagggagcagagaagaaggaggagttccaggatgagagtgagtttcgccgccagcatgaaggtgaggtcaaggccaaggagggcagaatggaagccaaggacggcagcctgaaggccaaggaaggaagcacggggaccagtgagagtagtgtagaggccaggggcagcgaggaggtcagagagagcagcacggtggccagcgacggcagcatggagggtaaggaaggcagcaccaaagttgaggagaacagcatgaaggcagacaagggtcgcacagaggtcaaccagtgcagcattgacttgggtgaagatgacatggagtttggtgaagacgacatcaatttcagtgaggatgacgtcgaggcagtgaacatcccggagagcctcccacccagtcgtcgtaacagcaacagcaaccctcctctgcccaggtgctaccagtgcaaagctgctaaagtgatcttcatcatcattttctcctatgtgctatccctggggccctactgctttttagcagtcctggccgtgtgggtggatgtcgaaacccaggtaccccagtgggtgatcaccataatcatctggcttttcttcctgcagtgctgcatccacccctatgtctatggctacatgcacaagaccattaagaaggaaatccaggacatgctgaagaagttcttctgcaaggaaaagcccccgaaagaagatagccacccagacctgcccggaacagagggtgggactgaaggcaagattgtcccttcctacgattctgctacttttcctgcgatatctgcagaattccaccacactggactagtggatccgagctcggtaccaagcttgggctgcaggtcgatgggctgcctcggcaacagtaagaccgaggaccagcgcaacgaggagaaggcgcagcgcgaggccaacaaaaagatcgagaagcagctgcagaaggacaagcaggtctaccgggccacgcaccgcctgctgctgctgggtgctggagagtctggcaaaagcaccattgtgaagcagatgaggatcctacatgttaatgggtttaacggagagggcggcgaagaggacccgcaggctgcaaggagcaacagcgatggtgagaaggccaccaaagtgcaggacatcaaaaacaacctgaaggaggccattgaaaccattgtggccgccatgagcaacctggtgccccccgtggagctggccaaccctgagaaccagttcagagtggactacattctgagcgtgatgaacgtgccaaactttgacttcccacctgaattctatgagcatgccaaggctctgtgggaggatgagggagttcgtgcctgctacgagcgctccaacgagtaccagctgatcgactgtgcccagtacttcctggacaagattgatgtgatcaagcaggccgactacgtgccaagtgaccaggacctgcttcgctgccgcgtcctgacctctggaatctttgagaccaagttccaggtggacaaagtcaacttccacatgttcgatgtgggcggccagcgcgatgaacgccgcaagtggatccagtgcttcaatgatgtgactgccatcatcttcgtggtggccagcagcagctacaacatggtcatccgggaggacaaccagaccaaccgtctgcaggaggctctgaacctcttcaagagcatctggaacaacagatggctgcgtaccatctctgtgatcctcttcctcaacaagcaagatctgcttgctgagaaggtcctcgctgggaaatcgaagattgaggactactttccagagttcgctcgctacaccactcctgaggatgcgactcccgagcccggagaggacccacgcgtgacccgggccaagtacttcatccgggatgagtttctgagaatcagcactgctagtggagatggacgtcactactgctaccctcactttacctgcgccgtggacactgagaacatccgccgtgtcttcaacgactgccgtgacatcatccagcgcatgcatcttcgccaatacgagctgctctaa

SEQ ID NO: 104 - protein, 926 aa, Homo sapiens and Rat [sequence omitted]

SEQ ID NO: 105 - DNA, 23 bp, Artificial Sequence
  misc_feature: Novel Sequence
catgtatgccagcgtcctgctcc

SEQ ID NO: 106 - DNA, 24 bp, Artificial Sequence
  misc_feature: Novel Sequence
gctatgcctgaagccagtcttgtg

SEQ ID NO: 107 - DNA, 25 bp, Artificial Sequence
  misc_feature: Novel Sequence
gcacctgctcctgagcaccttctcc

SEQ ID NO: 108 - DNA, 26 bp, Artificial Sequence
  misc_feature: Novel Sequence
cacagcgctgcagccctgcagctggc

SEQ ID NO: 109 - DNA, 24 bp, Artificial Sequence
  misc_feature: Novel Sequence
ccagtgatgactctgtccagcctg

SEQ ID NO: 110 - DNA, 24 bp, Artificial Sequence
  misc_feature: Novel Sequence
cagacacttggcagggacgaggtg

SEQ ID NO: 111 - DNA, 26 bp, Artificial Sequence
  misc_feature: Novel Sequence
cttgtggtctactgcagcatgttccg

SEQ ID NO: 112 - DNA, 25 bp, Artificial Sequence
  misc_feature: Novel Sequence
catatccctccgagtgtccagcggc

SEQ ID NO: 113 - DNA, 24 bp, Artificial Sequence
  misc_feature: Novel Sequence
atggatccttatcatggcttcctc

SEQ ID NO: 114 - DNA, 27 bp, Artificial Sequence
  misc_feature: Novel Sequence
caagaacaggtctcatctaagagctcc

SEQ ID NO: 115 - DNA, 26 bp, Artificial Sequence
  misc_feature: Novel Sequence
ctctgatgccatctgctggattcctg

SEQ ID NO: 116 - DNA, 26 bp, Artificial Sequence
  misc_feature: Novel Sequence
gtagtccactgaaagtccagtgatcc

SEQ ID NO: 117 - DNA, 24 bp, Artificial Sequence
  misc_feature: Novel Sequence
tggtggcgatggccaacagcgctc

SEQ ID NO: 118 - DNA, 24 bp, Artificial Sequence
  misc_feature: Novel Sequence
gttgcgccttagcgacagatgacc

SEQ ID NO: 119 - DNA, 23 bp, Artificial Sequence
  misc_feature: Novel Sequence
tcaacctgtatagcagcatcctc

SEQ ID NO: 120 - DNA, 23 bp, Artificial Sequence
  misc_feature: Novel Sequence
aaggagtagcagaatggttagcc

SEQ ID NO: 121 - DNA, 24 bp, Artificial Sequence
  misc_feature: Novel Sequence
gacacctgtcagcggtcgtgtgtg

SEQ ID NO: 122 - DNA, 27 bp, Artificial Sequence
  misc_feature: Novel Sequence
ctgatggaagtagaggctgtccatctc

SEQ ID NO: 123 - DNA, 24 bp, Artificial Sequence
  misc_feature: Novel Sequence
gcgctgagcgcagaccagtggctg

SEQ ID NO: 124 - DNA, 24 bp, Artificial Sequence
  misc_feature: Novel Sequence
cacggtgacgaagggcacgagctc

SEQ ID NO: 125 - DNA, 24 bp, Artificial Sequence
  misc_feature: Novel Sequence
agccatccctgccaggaagcatgg

SEQ ID NO: 126 - DNA, 25 bp, Artificial Sequence
  misc_feature: Novel Sequence
ccaggtaggtgtgcagcacaatggc

SEQ ID NO: 127 - DNA, 25 bp, Artificial Sequence
  misc_feature: Novel Sequence
ctgttcaacagggctggttggcaac

SEQ ID NO: 128 - DNA, 25 bp, Artificial Sequence
  misc_feature: Novel Sequence
atcatgtctagactcatggtgatcc

SEQ ID NO: 129 - protein, 6 aa, Artificial Sequence
  misc_feature: Novel Sequence
TLESIM

SEQ ID NO: 130 - protein, 5 aa, Artificial Sequence
  misc_feature: Novel Sequence
EYNLV

SEQ ID NO: 131 - protein, 5 aa, Artificial Sequence
  misc_feature: Novel Sequence
DCGLF

SEQ ID NO: 132 - protein, 36 aa, Artificial Sequence
  misc_feature: Novel Sequence
GATCAAGCTTCCATGGCGTGCTGCCTGAGCGAGGAG

SEQ ID NO: 133 - protein, 53 aa, Artificial Sequence
  misc_feature: Novel Sequence
GATCGGATCCTTAGAACAGGCCGCAGTCCTTCAGGTTCAGCTGCAGGATGGTG

(19) United States
(12) Patent Application Publication
(10) Pub. No.: US 2013/0165633 A1
(43) Pub. Date: Jun. 27, 2013

(54) Endogenous and Non-Endogenous Versions of Human G Protein-Coupled Receptors

(71) Applicants: RUOPING CHEN, SAN DIEGO, CA (US); HUONG T. DANG, SAN DIEGO, CA (US); KEVIN P. LOWITZ, SAN DIEGO, CA (US)

(72) Inventors: RUOPING CHEN, SAN DIEGO, CA (US); HUONG T. DANG, SAN DIEGO, CA (US); KEVIN P. LOWITZ, SAN DIEGO, CA (US)

(21) Appl. No.: 13/494,750

(22) Filed: Jun. 12, 2012

Related U.S. Application Data
(63) Continuation of application No. 12/536,371, filed on Aug. 5, 2009, now abandoned.
Application No. 12/536,371 is a continuation of application No. 10/321,807, filed on Dec. 16, 2002, now abandoned.
Application No. 10/321,807 is a continuation of application No. 09/714,008, filed on Nov. 16, 2000, now abandoned.
(60) Provisional application No. 60/166,088, filed on Nov. 17, 1999.
(60) Provisional application No. 60/166,099, filed on Nov. 17, 1999.
(60) Provisional application No. 60/166,369, filed on Nov. 17, 1999.
(60) Provisional application No. 60/171,902, filed on Dec. 23, 1999.
(60) Provisional application No. 60/171,901, filed on Dec. 23, 1999.
(60) Provisional application No. 60/171,900, filed on Dec. 23, 1999.
(60) Provisional application No. 60/181,749, filed on Feb. 11, 2000.
(60) Provisional application No. 60/189,258, filed on Mar. 14, 2000.
(60) Provisional application No. 60/189,259, filed on Mar. 14, 2000.
(60) Provisional application No. 60/195,899, filed on Apr. 10, 2000.
(60) Provisional application No. 60/196,078, filed on Apr. 10, 2000.
(60) Provisional application No. 60/195,898, filed on Apr. 10, 2000.
(60) Provisional application No. 60/200,419, filed on Apr. 28, 2000.
(60) Provisional application No. 60/203,630, filed on May 12, 2000.
(60) Provisional application No. 60/210,741, filed on Jun. 12, 2000.
(60) Provisional application No. 60/210,982, filed on Jun. 12, 2000.
(60) Provisional application No. 60/226,760, filed on Aug. 21, 2000.
(60) Provisional application No. 60/235,779, filed on Sep. 26, 2000.
(60) Provisional application No. 60/235,418, filed on Sep. 26, 2000.
(60) Provisional application No. 60/242,332, filed on Oct. 20, 2000.
(60) Provisional application No. 60/242,343, filed on Oct. 20, 2000.
(60) Provisional application No. 60/243,019, filed on Oct. 24, 2000.

(51) Int. Cl. C07K 14/705

(52) U.S. Cl. CPC C07K 14/705
USPC 530/350; 536/23.5; 435/320.1; 435/325; 435/455; 435/29; 435/365

(57) ABSTRACT

The invention disclosed in this patent document relates to transmembrane receptors, more particularly to a human G protein-coupled receptor for which the endogenous ligand is unknown (“orphan GPCR receptors”), and most particularly to mutated (non-endogenous) versions of the human GPCRs for evidence of constitutive activity.

This application is a continuation-in-part of U.S. Ser. No. 09/170,496, filed with the United States Patent and Trademark Office on Oct. 13, 1998 and its corresponding PCT application number PCT/US99/23938, published as WO 00/22129 on Apr. 20, 2000. This document claims the benefit of priority from the following provisional applications, all filed via U.S. Express Mail with the United States Patent and Trademark Office on the indicated dates: U.S. Provisional No. 60/166,088, filed Nov. 17, 1999; U.S. Provisional No. 60/166,369, filed Nov. 17, 1999; U.S. Provisional No. 60/166,099 filed Nov. 17, 1999; U.S. Provisional No. 60/171,902, filed Dec. 23, 1999; U.S. Provisional No. 60/171,901, filed Dec. 23, 1999; U.S. Provisional No. 60/171,900, filed Dec. 23, 1999; U.S. Provisional No. 60/181,749, filed Feb. 11, 2000; U.S. Provisional No. 60/189,258, filed Mar. 14, 2000; U.S. Provisional No. 60/189,259, filed Mar. 14, 2000; U.S. Provisional No. 60/195,899, filed Apr. 10, 2000; U.S. Provisional No. 60/196,078, filed Apr. 10, 2000; U.S. Provisional No. 60/195,898, filed Apr. 10, 2000; U.S. Provisional No. 60/200,419, filed Apr. 28, 2000; U.S. Provisional No. 60/203,630, filed May 12, 2000; U.S. Provisional No. 60/210,741, filed Jun. 12, 2000; U.S. Provisional No. 60/210,982, filed Jun. 12, 2000; U.S. Provisional No. 60/226,760, filed Aug. 21, 2000, claiming priority from U.S. Provisional No. 60/171,900, filed Dec. 23, 1999; U.S. Provisional No. 60/235,779, filed Sep. 26, 2000; U.S. Provisional No. 60/235,418, filed Sep. 26, 2000; U.S. Provisional No. 60/242,332, filed Oct. 20, 2000; and U.S. Provisional No. 60/243,019, filed Oct. 24, 2000 claiming priority from U.S. Provisional No. 60/242,343, filed Oct. 20, 2000.

### FIELD OF THE INVENTION

The invention disclosed in this patent document relates to transmembrane receptors, and more particularly to human G protein-coupled receptors, and specifically to endogenous human GPCRs with particular emphasis on non-endogenous versions of the GPCRs that have been altered to establish or enhance constitutive activity of the receptor. Preferably, the altered GPCRs are used for the direct identification of candidate compounds as receptor agonists, inverse agonists or partial agonists having potential applicability as therapeutic agents.

### BACKGROUND OF THE INVENTION

Although a number of receptor classes exist in humans, by far the most abundant and therapeutically relevant is represented by the G protein-coupled receptor (GPCR or GPCRs) class. It is estimated that there are some 100,000 genes within the human genome, and of these, approximately 2%, or 2,000 genes, are estimated to code for GPCRs. Receptors, including GPCRs, for which the endogenous ligand has been identified are referred to as “known” receptors, while receptors for which the endogenous ligand has not been identified are referred to as “orphan” receptors. GPCRs represent an important area for the development of pharmaceutical products: from approximately 20 of the 100 known GPCRs, approximately 60% of all prescription pharmaceuticals have been developed.

GPCRs share a common structural motif. All these receptors have seven sequences of between 22 to 24 hydrophobic amino acids that form seven alpha helices, each of which spans the membrane (each span is identified by number, i.e., transmembrane-1 (TM-1), transmebrane-2 (TM-2), etc.). The transmembrane helices are joined by strands of amino acids between transmembrane-2 and transmembrane-3, transmembrane-4 and transmembrane-5, and transmembrane-6 and transmembrane-7 on the exterior, or “extracellular” side, of the cell membrane (these are referred to as “extracellular” regions 1, 2 and 3 (EC-1, EC-2 and EC-3), respectively). The transmembrane helices are also joined by strands of amino acids between transmembrane-1 and transmembrane-2, transmembrane-3 and transmembrane-4, and transmembrane-5 and transmembrane-6 on the interior, or “intracellular” side, of the cell membrane (these are referred to as “intracellular” regions 1, 2 and 3 (IC-1, IC-2 and IC-3), respectively). The “carboxy” (“C”) terminus of the receptor lies in the intracellular space within the cell, and the “amino” (“N”) terminus of the receptor lies in the extracellular space outside of the cell.

Generally, when an endogenous ligand binds with the receptor (often referred to as “activation” of the receptor), there is a change in the conformation of the intracellular region that allows for coupling between the intracellular region and an intracellular “G-protein.” It has been reported that GPCRs are “promiscuous” with respect to G proteins, i.e., that a GPCR can interact with more than one G protein. See, Kenakin, T., 43 Life Sciences 1095 (1988). Although other G proteins exist, currently, Gq, Gs, Gi, Gz and Go are G proteins that have been identified. Endogenous ligand-activated GPCR coupling with the G-protein begins a signaling cascade process (referred to as “signal transduction”). Under normal conditions, signal transduction ultimately results in cellular activation or cellular inhibition. It is thought that the IC-3 loop as well as the carboxy terminus of the receptor interact with the G protein.

Under physiological conditions, GPCRs exist in the cell membrane in equilibrium between two different conformations: an “inactive” state and an “active” state. A receptor in an inactive state is unable to link to the intracellular signaling transduction pathway to produce a biological response. Changing the receptor conformation to the active state allows linkage to the transduction pathway (via the G-protein) and produces a biological response.

A receptor may be stabilized in an active state by an endogenous ligand or a compound such as a drug. Recent discoveries, including but not exclusively limited to modifications to the amino acid sequence of the receptor, provide means other than endogenous ligands or drugs to promote and stabilize the receptor in the active state conformation. These means effectively stabilize the receptor in an active state by simulating the effect of an endogenous ligand binding to the receptor. Stabilization by such ligand-independent means is termed “constitutive receptor activation.”

### SUMMARY OF THE INVENTION

Disclosed herein are endogenous and non-endogenous versions of human GPCRs and uses thereof.

### BRIEF DESCRIPTION OF THE DRAWINGS

FIG. 1 provides an illustration of second messenger IP₃ production from endogenous version RUP12 (“RUP12”) as compared with the control (“CMV”).

FIG. 2 is a graphic representation of the results of a second messenger cell-based cyclic AMP assay providing comparative results for constitutive signaling of endogenous RUP13 (“RUP13”) and a control vector (“CMV”).

FIG. 3 is a diagrammatic representation of the signal measured comparing CMV, endogenous RUP13 (“RUP13 wt”) and non-endogenous, constitutively activated RUP13 (“RUP13(A268K)”), utilizing 8×CRE-Luc reporter plasmid.

FIG. 4 is a graphic representation of the results of a [³⁵S]GTPγS assay providing comparative results for constitutive signaling by RUP13:Gs Fusion Protein (“RUP13-Gs”) and a control vector (“CMV”).

FIG. 5 is a diagrammatic representation of the signal measured comparing CMV, endogenous RUP14 (“RUP14 wt”) and non-endogenous, constitutively activated RUP13 (“RUP14(L246K)”), utilizing 8×CRE-Luc reporter plasmid.

FIG. 6 is a diagrammatic representation of the signal measured comparing CMV, endogenous RUP15 (“RUP15 wt”) and non-endogenous, constitutively activated RUP15 (“RUP15(A398K)”), utilizing 8×CRE-Luc reporter plasmid.

FIG. 7 is a graphic representation of the results of a second messenger cell-based cyclic AMP assay providing comparative results for constitutive signaling of endogenous RUP15 (“RUP15 wt”), non-endogenous, constitutively activated version of RUP15 (“RUP15(A398K)”) and a control vector (“CMV”).

FIG. 8 is a graphic representation of the results of a [³⁵S]GTPγS assay providing comparative results for constitutive signaling by RUP15:Gs Fusion Protein (“RUP15-Gs”) and a control vector (“CMV”).

FIG. 9 provides an illustration of second messenger IP₃ production from endogenous version RUP17 (“RUP17”) as compared with the control (“CMV”).

FIG. 10 provides an illustration of second messenger IP₃ production from endogenous version RUP21 (“RUP21”) as compared with the control (“CMV”).

FIG. 11 is a diagrammatic representation of the signal measured comparing CMV, endogenous RUP23 (“RUP23 wt”) and non-endogenous, constitutively activated RUP23 (“RUP23(W275K)”), utilizing 8×CRE-Luc reporter plasmid.

FIG. 12 is a graphic representation of results from a primary screen of several candidate compounds against RUP13; results for “Compound A” are provided in well A2 and “Compound “B” are provided in well G9.

### DETAILED DESCRIPTION

The scientific literature that has evolved around receptors has adopted a number of terms to refer to ligands having various effects on receptors. For clarity and consistency, the following definitions will be used throughout this patent document. To the extent that these definitions conflict with other definitions for these terms, the following definitions shall control:

AGONISTS shall mean materials (e.g., ligands, candidate compounds) that activate the intracellular response when they bind to the receptor, or enhance GTP binding to membranes.

AMINO ACID ABBREVIATIONS used herein are set out in Table A:

TABLE A

| | | |
|---|---|---|
| ALANINE | ALA | A |
| ARGININE | ARG | R |
| ASPARAGINE | ASN | N |
| ASPARTIC ACID | ASP | D |
| CYSTEINE | CYS | C |
| GLUTAMIC ACID | GLU | E |
| GLUTAMINE | GLN | Q |
| GLYCINE | GLY | G |
| HISTIDINE | HIS | H |
| ISOLEUCINE | ILE | I |
| LEUCINE | LEU | L |
| LYSINE | LYS | K |
| METHIONINE | MET | M |
| PHENYLALANINE | PHE | F |
| PROLINE | PRO | P |
| SERINE | SER | S |
| THREONINE | THR | T |
| TRYPTOPHAN | TRP | W |
| TYROSINE | TYR | Y |
| VALINE | VAL | V |

PARTIAL AGONISTS shall mean materials (e.g., ligands, candidate compounds) that activate the intracellular response when they bind to the receptor to a lesser degree/extent than do agonists, or enhance GTP binding to membranes to a lesser degree/extent than do agonists.

ANTAGONIST shall mean materials (e.g., ligands, candidate compounds) that competitively bind to the receptor at the same site as the agonists but which do not activate the intracellular response initiated by the active form of the receptor, and can thereby inhibit the intracellular responses by agonists or partial agonists. ANTAGONISTS do not diminish the baseline intracellular response in the absence of an agonist or partial agonist.

CANDIDATE COMPOUND shall mean a molecule (for example, and not limitation, a chemical compound) that is amenable to a screening technique. Preferably, the phrase “candidate compound” does not include compounds which were publicly known to be compounds selected from the group consisting of inverse agonist, agonist or antagonist to a receptor, as previously determined by an indirect identification process (“indirectly identified compound”); more preferably, not including an indirectly identified compound which has previously been determined to have therapeutic efficacy in at least one mammal; and, most preferably, not including an indirectly identified compound which has previously been determined to have therapeutic utility in humans.

COMPOSITION means a material comprising at least one component; a “pharmaceutical composition” is an example of a composition.

COMPOUND EFFICACY shall mean a measurement of the ability of a compound to inhibit or stimulate receptor functionality, as opposed to receptor binding affinity. Exemplary means of detecting compound efficacy are disclosed in the Example section of this patent document.

CODON shall mean a grouping of three nucleotides (or equivalents to nucleotides) which generally comprise a nucleoside (adenosine (A), guanosine (G), cytidine (C), uridine (U) and thymidine (T)) coupled to a phosphate group and which, when translated, encodes an amino acid.

CONSTITUTIVELY ACTIVATED RECEPTOR shall mean a receptor subject to constitutive receptor activation. A constitutively activated receptor can be endogenous or non-endogenous.

CONSTITUTIVE RECEPTOR ACTIVATION shall mean stabilization of a receptor in the active state by means other than binding of the receptor with its endogenous ligand or a chemical equivalent thereof.

CONTACT or CONTACTING shall mean bringing at least two moieties together, whether in an in vitro system or an in vivo system.

DIRECTLY IDENTIFYING or DIRECTLY IDENTIFIED, in relationship to the phrase “candidate compound”, shall mean the screening of a candidate compound against a constitutively activated receptor, preferably a constitutively activated orphan receptor, and most preferably against a constitutively activated G protein-coupled cell surface orphan receptor, and assessing the compound efficacy of such compound. This phrase is, under no circumstances, to be interpreted or understood to be encompassed by or to encompass the phrase “indirectly identifying” or “indirectly identified.”

ENDOGENOUS shall mean a material that a mammal naturally produces. ENDOGENOUS in reference to, for example and not limitation, the term “receptor,” shall mean that which is naturally produced by a mammal (for example, and not limitation, a human) or a virus. By contrast, the term NON-ENDOGENOUS in this context shall mean that which is not naturally produced by a mammal (for example, and not limitation, a human) or a virus. For example, and not limitation, a receptor which is not constitutively active in its endogenous form, but when manipulated becomes constitutively active, is most preferably referred to herein as a “non-endogenous, constitutively activated receptor.” Both terms can be utilized to describe both “in vivo” and “in vitro” systems. For example, and not limitation, in a screening approach, the endogenous or non-endogenous receptor may be in reference to an in vitro screening system. As a further example and not limitation, where the genome of a mammal has been manipulated to include a non-endogenous constitutively activated receptor, screening of a candidate compound by means of an in vivo system is viable.

G PROTEIN COUPLED RECEPTOR FUSION PROTEIN and GPCR FUSION PROTEIN, in the context of the invention disclosed herein, each mean a non-endogenous protein comprising an endogenous, constitutively activate GPCR or a non-endogenous, constitutively activated GPCR fused to at least one G protein, most preferably the alpha (α) subunit of such G protein (this being the subunit that binds GTP), with the G protein preferably being of the same type as the G protein that naturally couples with endogenous orphan GPCR. For example, and not limitation, in an endogenous state, if the G protein “Gsα” is the predominate G protein that couples with the GPCR, a GPCR Fusion Protein based upon the specific GPCR would be a non-endogenous protein comprising the GPCR fused to Gsα; in some circumstances, as will be set forth below, a non-predominant G protein can be fused to the GPCR. The G protein can be fused directly to the c-terminus of the constitutively active GPCR or there may be spacers between the two.

HOST CELL shall mean a cell capable of having a Plasmid and/or Vector incorporated therein. In the case of a prokaryotic Host Cell, a Plasmid is typically replicated as a autonomous molecule as the Host Cell replicates (generally, the Plasmid is thereafter isolated for introduction into a eukaryotic Host Cell); in the case of a eukaryotic Host Cell, a Plasmid is integrated into the cellular DNA of the Host Cell such that when the eukaryotic Host Cell replicates, the Plasmid replicates. Preferably, for the purposes of the invention disclosed herein, the Host Cell is eukaryotic, more preferably, mammalian, and most preferably selected from the group consisting of 293, 293T and COS-7 cells.

INDIRECTLY IDENTIFYING or INDIRECTLY IDENTIFIED means the traditional approach to the drug discovery process involving identification of an endogenous ligand specific for an endogenous receptor, screening of candidate compounds against the receptor for determination of those which interfere and/or compete with the ligand-receptor interaction, and assessing the efficacy of the compound for affecting at least one second messenger pathway associated with the activated receptor.

INHIBIT or INHIBITING, in relationship to the term “response” shall mean that a response is decreased or prevented in the presence of a compound as opposed to in the absence of the compound.

INVERSE AGONISTS shall mean materials (e.g., ligand, candidate compound) which bind to either the endogenous form of the receptor or to the constitutively activated form of the receptor, and which inhibit the baseline intracellular response initiated by the active form of the receptor below the normal base level of activity which is observed in the absence of agonists or partial agonists, or decrease GTP binding to membranes. Preferably, the baseline intracellular response is inhibited in the presence of the inverse agonist by at least 30%, more preferably by at least 50%, and most preferably by at least 75%, as compared with the baseline response in the absence of the inverse agonist.

KNOWN RECEPTOR shall mean an endogenous receptor for which the endogenous ligand specific for that receptor has been identified.

LIGAND shall mean an endogenous, naturally occurring molecule specific for an endogenous, naturally occurring receptor.

MUTANT or MUTATION in reference to an endogenous receptor's nucleic acid and/or amino acid sequence shall mean a specified change or changes to such endogenous sequences such that a mutated form of an endogenous, non-constitutively activated receptor evidences constitutive activation of the receptor. In terms of equivalents to specific sequences, a subsequent mutated form of a human receptor is considered to be equivalent to a first mutation of the human receptor if (a) the level of constitutive activation of the subsequent mutated form of a human receptor is substantially the same as that evidenced by the first mutation of the receptor; and (b) the percent sequence (amino acid and/or nucleic acid) homology between the subsequent mutated form of the receptor and the first mutation of the receptor is at least about 80%, more preferably at least about 90% and most preferably at least 95%. Ideally, and owing to the fact that the most preferred cassettes disclosed herein for achieving constitutive activation includes a single amino acid and/or codon change between the endogenous and the non-endogenous forms of the GPCR, the percent sequence homology should be at least 98%.

NON-ORPHAN RECEPTOR shall mean an endogenous naturally occurring molecule specific for an endogenous naturally occurring ligand wherein the binding of a ligand to a receptor activates an intracellular signaling pathway.

ORPHAN RECEPTOR shall mean an endogenous receptor for which the endogenous ligand specific for that receptor has not been identified or is not known.

PHARMACEUTICAL COMPOSITION shall mean a composition comprising at least one active ingredient, whereby the composition is amenable to investigation for a specified, efficacious outcome in a mammal (for example, and not limitation, a human). Those of ordinary skill in the art will understand and appreciate the techniques appropriate for determining whether an active ingredient has a desired efficacious outcome based upon the needs of the artisan.

PLASMID shall mean the combination of a Vector and cDNA. Generally, a Plasmid is introduced into a Host Cell for the purposes of replication and/or expression of the cDNA as a protein.

SECOND MESSENGER shall mean an intracellular response produced as a result of receptor activation. A second messenger can include, for example, inositol triphosphate (IP₃), diacycglycerol (DAG), cyclic AMP (cAMP), and cyclic GMP (cGMP). Second messenger response can be measured for a determination of receptor activation. In addition, second messenger response can be measured for the direct identification of candidate compounds, including for example, inverse agonists, agonists, partial agonists and antagonists.

STIMULATE or STIMULATING, in relationship to the term “response” shall mean that a response is increased in the presence of a compound as opposed to in the absence of the compound.

VECTOR in reference to cDNA shall mean a circular DNA capable of incorporating at least one cDNA and capable of incorporation into a Host Cell.

The order of the following sections is set forth for presentational efficiency and is not intended, nor should be construed, as a limitation on the disclosure or the claims to follow.

### A. Introduction

The traditional study of receptors has always proceeded from the a priori assumption (historically based) that the endogenous ligand must first be identified before discovery could proceed to find antagonists and other molecules that could affect the receptor. Even in cases where an antagonist might have been known first, the search immediately extended to looking for the endogenous ligand. This mode of thinking has persisted in receptor research even after the discovery of constitutively activated receptors. What has not been heretofore recognized is that it is the active state of the receptor that is most useful for discovering agonists, partial agonists, and inverse agonists of the receptor. For those diseases which result from an overly active receptor or an under-active receptor, what is desired in a therapeutic drug is a compound which acts to diminish the active state of a receptor or enhance the activity of the receptor, respectively, not necessarily a drug which is an antagonist to the endogenous ligand. This is because a compound that reduces or enhances the activity of the active receptor state need not bind at the same site as the endogenous ligand. Thus, as taught by a method of this invention, any search for therapeutic compounds should start by screening compounds against the ligand-independent active state.

### B. Identification of Human GPCRs

The efforts of the Human Genome project has led to the identification of a plethora of information regarding nucleic acid sequences located within the human genome; it has been the case in this endeavor that genetic sequence information has been made available without an understanding or recognition as to whether or not any particular genomic sequence does or may contain open-reading frame information that translate human proteins. Several methods of identifying nucleic acid sequences within the human genome are within the purview of those having ordinary skill in the art. For example, and not limitation, a variety of human GPCRs, disclosed herein, were discovered by reviewing the GenBank™ database. Table B, below, lists several endogenous GPCRs that we have discovered, along with other GPCR's that are homologous to the disclosed GPCR.

TABLE B

| Disclosed | | Open | | Per Cent |
|---|---|---|---|---|
| Human | Accession | Reading | Reference To | Homology |
| Orphan | Number | Frame | Homologous | To Designated |
| GPCRs | Identified | (Base Pairs) | GPCR | GPCR |
| hRUP8 | AL121755 | 1,152 bp | NPY2R | 27% |
| hRUP9 | AC0113375 | 1,260 bp | GAL2R | 22% |
| hRUP10 | AC008745 | 1,014 bp | C5aR | 40% |
| hRUP11 | AC013396 | 1,272 bp | HM74 | 36% |
| hRUP12 | AP000808 | 966 bp | Mas1 | 34% |
| hRUP13 | AC011780 | 1,356 bp | Fish GPRX- | 43% |
| | | | ORYLA | |
| hRUP14 | AL137118 | 1,041 bp | CysLT1R | 35% |
| hRUP15 | AL016468 | 1,527 bp | RE2 | 30% |
| hRUP16 | AL136106 | 1,068 bp | GLR101 | 37% |
| hRUP17 | AC023078 | 969 bp | Mas1 | 37% |
| hRUP18 | AC008547 | 1,305 bp | Oxytocin | 31% |
| hRUP19 | AC026331 | 1,041 bp | HM74 | 52% |
| hRUP20 | AL161458 | 1,011 bp | GPR34 | 25% |
| hRUP21 | AC026756 | 1,014 bp | P2Y1R | 37% |
| hRUP22 | AC027026 | 993 bp | RUP17 | 67% |
| | | | Mas1 | 37% |
| hRUP23 | AC007104 | 1,092 bp | Rat GPR26 | 31% |
| hRUP24 | AL355388 | 1,125 bp | SALPR | 44% |
| hRUP25 | AC026331 | 1,092 bp | HM74 | 95% |
| hRUP26 | AC023040 | 1,044 bp | Rabbit 5HT1D | 27% |
| hRUP27 | AC027643 | 158,700 | MCH | 38% |

Receptor homology is useful in terms of gaining an appreciation of a role of the receptors within the human body. As the patent document progresses, we will disclose techniques for mutating these receptors to establish non-endogenous, constitutively activated versions of these receptors.

The techniques disclosed herein have also been applied to other human, orphan GPCRs known to the art, as will be apparent as the patent document progresses.

### C. Receptor Screening

Screening candidate compounds against a non-endogenous, constitutively activated version of the human GPCRs disclosed herein allows for the direct identification of candidate compounds which act at this cell surface receptor, without requiring use of the receptor's endogenous ligand. Using routine, and often commercially available techniques, one can determine areas within the body where the endogenous version of human GPCRs disclosed herein is expressed and/or over-expressed. It is also possible using these techniques to determine related disease/disorder states which are associated with the expression and/or over-expression of the receptor; such an approach is disclosed in this patent document.

With respect to creation of a mutation that may evidence constitutive activation of the human GPCR disclosed herein is based upon the distance from the proline residue at which is presumed to be located within TM6 of the GPCR; this algorithmic technique is disclosed in co-pending and commonly assigned patent document PCT Application Number PCT/US99/23938, published as WO 00/22129 on Apr. 20, 2000, which, along with the other patent documents listed herein, is incorporated herein by reference. The algorithmic technique is not predicated upon traditional sequence “alignment” but rather a specified distance from the aforementioned TM6 proline residue (or, of course, endogenous constitutive substitution for such proline residue). By mutating the amino acid residue located 16 amino acid residues from this residue (presumably located in the IC3 region of the receptor) to, most preferably, a lysine residue, such activation may be obtained. Other amino acid residues may be useful in the mutation at this position to achieve this objective.

D. Disease/Disorder Identification and/or Selection

As will be set forth in greater detail below, most preferably inverse agonists and agonists to the non-endogenous, constitutively activated GPCR can be identified by the methodologies of this invention. Such inverse agonists and agonists are ideal candidates as lead compounds in drug discovery programs for treating diseases related to this receptor. Because of the ability to directly identify inverse agonists to the GPCR, thereby allowing for the development of pharmaceutical compositions, a search for diseases and disorders associated with the GPCR is relevant. For example, scanning both diseased and normal tissue samples for the presence of the GPCR now becomes more than an academic exercise or one which might be pursued along the path of identifying an endogenous ligand to the specific GPCR. Tissue scans can be conducted across a broad range of healthy and diseased tissues. Such tissue scans provide a preferred first step in associating a specific receptor with a disease and/or disorder.

Preferably, the DNA sequence of the human GPCR is used to make a probe for (a) dot-blot analysis against tissue-mRNA, and/or (b) RT-PCR identification of the expression of the receptor in tissue samples. The presence of a receptor in a tissue source, or a diseased tissue, or the presence of the receptor at elevated concentrations in diseased tissue compared to a normal tissue, can be preferably utilized to identify a correlation with a treatment regimen, including but not limited to, a disease associated with that disease. Receptors can equally well be localized to regions of organs by this technique. Based on the known functions of the specific tissues to which the receptor is localized, the putative functional role of the receptor can be deduced.

### E. Screening of Candidate Compounds

1. Generic GPCR Screening Assay Techniques

When a G protein receptor becomes constitutively active, it binds to a G protein (e.g., Gq, Gs, Gi, Gz, Go) and stimulates the binding of GTP to the G protein. The G protein then acts as a GTPase and slowly hydrolyzes the GTP to GDP, whereby the receptor, under normal conditions, becomes deactivated. However, constitutively activated receptors continue to exchange GDP to GTP. A non-hydrolyzable analog of GTP, [³⁵S]GTPγS, can be used to monitor enhanced binding to membranes which express constitutively activated receptors. It is reported that [³⁵S]GTPγS can be used to monitor G protein coupling to membranes in the absence and presence of ligand. An example of this monitoring, among other examples well-known and available to those in the art, was reported by Traynor and Nahorski in 1995. The preferred use of this assay system is for initial screening of candidate compounds because the system is generically applicable to all G protein-coupled receptors regardless of the particular G protein that interacts with the intracellular domain of the receptor.

2. Specific GPCR Screening Assay Techniques

Once candidate compounds are identified using the “generic” G protein-coupled receptor assay (i.e., an assay to select compounds that are agonists, partial agonists, or inverse agonists), further screening to confirm that the compounds have interacted at the receptor site is preferred. For example, a compound identified by the “generic” assay may not bind to the receptor, but may instead merely “uncouple” the G protein from the intracellular domain.

a. Gs, Gz and Gi.

Gs stimulates the enzyme adenylyl cyclase. Gi (and Gz and Go), on the other hand, inhibit this enzyme. Adenylyl cyclase catalyzes the conversion of ATP to cAMP; thus, constitutively activated GPCRs that couple the Gs protein are associated with increased cellular levels of cAMP. On the other hand, constitutively activated GPCRs that couple Gi (or Gz, Go) protein are associated with decreased cellular levels of cAMP. See, generally, “Indirect Mechanisms of Synaptic Transmission,” Chpt. 8, From Neuron To Brain (3^{rd} Ed.) Nichols, J. G. et al eds. Sinauer Associates, Inc. (1992). Thus, assays that detect cAMP can be utilized to determine if a candidate compound is, e.g., an inverse agonist to the receptor (i.e., such a compound would decrease the levels of cAMP). A variety of approaches known in the art for measuring cAMP can be utilized; a most preferred approach relies upon the use of anti-cAMP antibodies in an ELISA-based format. Another type of assay that can be utilized is a whole cell second messenger reporter system assay. Promoters on genes drive the expression of the proteins that a particular gene encodes. Cyclic AMP drives gene expression by promoting the binding of a cAMP-responsive DNA binding protein or transcription factor (CREB) that then binds to the promoter at specific sites called cAMP response elements and drives the expression of the gene. Reporter systems can be constructed which have a promoter containing multiple cAMP response elements before the reporter gene, e.g., β-galactosidase or luciferase. Thus, a constitutively activated Gs-linked receptor causes the accumulation of cAMP that then activates the gene and expression of the reporter protein. The reporter protein such as β-galactosidase or luciferase can then be detected using standard biochemical assays (Chen et al. 1995).

b. Go and Gq.

Gq and Go are associated with activation of the enzyme phospholipase C, which in turn hydrolyzes the phospholipid PIP₂, releasing two intracellular messengers: diacycloglycerol (DAG) and inistol 1,4,5-triphoisphate (IP₃). Increased accumulation of IP₃ is associated with activation of Gq- and Go-associated receptors. See, generally, “Indirect Mechanisms of Synaptic Transmission,” Chpt. 8, From Neuron To Brain (3^{rd} Ed.) Nichols, J. G. et al eds. Sinauer Associates, Inc. (1992). Assays that detect IP₃ accumulation can be utilized to determine if a candidate compound is, e.g., an inverse agonist to a Gq- or Go-associated receptor (i.e., such a compound would decrease the levels of IP₃). Gq-associated receptors can also been examined using an AP1 reporter assay in that Gq-dependent phospholipase C causes activation of genes containing AP1 elements; thus, activated Gq-associated receptors will evidence an increase in the expression of such genes, whereby inverse agonists thereto will evidence a decrease in such expression, and agonists will evidence an increase in such expression. Commercially available assays for such detection are available.

3. GPCR Fusion Protein

The use of an endogenous, constitutively activate orphan GPCR or a non-endogenous, constitutively activated orphan GPCR, for use in screening of candidate compounds for the direct identification of inverse agonists, agonists and partial agonists provide an interesting screening challenge in that, by definition, the receptor is active even in the absence of an endogenous ligand bound thereto. Thus, in order to differentiate between, e.g., the non-endogenous receptor in the presence of a candidate compound and the non-endogenous receptor in the absence of that compound, with an aim of such a differentiation to allow for an understanding as to whether such compound may be an inverse agonist, agonist, partial agonist or have no affect on such a receptor, it is preferred that an approach be utilized that can enhance such differentiation. A preferred approach is the use of a GPCR Fusion Protein.

Generally, once it is determined that a non-endogenous orphan GPCR has been constitutively activated using the assay techniques set forth above (as well as others), it is possible to determine the predominant G protein that couples with the endogenous GPCR. Coupling of the G protein to the GPCR provides a signaling pathway that can be assessed. Because it is most preferred that screening take place by use of a mammalian expression system, such a system will be expected to have endogenous G protein therein. Thus, by definition, in such a system, the non-endogenous, constitutively activated orphan GPCR will continuously signal. In this regard, it is preferred that this signal be enhanced such that in the presence of, e.g., an inverse agonist to the receptor, it is more likely that it will be able to more readily differentiate, particularly in the context of screening, between the receptor when it is contacted with the inverse agonist.

The GPCR Fusion Protein is intended to enhance the efficacy of G protein coupling with the non-endogenous GPCR. The GPCR Fusion Protein is preferred for screening with a non-endogenous, constitutively activated GPCR because such an approach increases the signal that is most preferably utilized in such screening techniques. This is important in facilitating a significant “signal to noise” ratio; such a significant ratio is import preferred for the screening of candidate compounds as disclosed herein.

The construction of a construct useful for expression of a GPCR Fusion Protein is within the purview of those having ordinary skill in the art. Commercially available expression vectors and systems offer a variety of approaches that can fit the particular needs of an investigator. The criteria of importance for such a GPCR Fusion Protein construct is that the endogenous GPCR sequence and the G protein sequence both be in-frame (preferably, the sequence for the endogenous GPCR is upstream of the G protein sequence) and that the “stop” codon of the GPCR must be deleted or replaced such that upon expression of the GPCR, the G protein can also be expressed. The GPCR can be linked directly to the G protein, or there can be spacer residues between the two (preferably, no more than about 12, although this number can be readily ascertained by one of ordinary skill in the art). We have a preference (based upon convenience) of use of a spacer in that some restriction sites that are not used will, effectively, upon expression, become a spacer. Most preferably, the G protein that couples to the non-endogenous GPCR will have been identified prior to the creation of the GPCR Fusion Protein construct. Because there are only a few G proteins that have been identified, it is preferred that a construct comprising the sequence of the G protein (i.e., a universal G protein construct) be available for insertion of an endogenous GPCR sequence therein; this provides for efficiency in the context of large-scale screening of a variety of different endogenous GPCRs having different sequences.

As noted above, constitutively activated GPCRs that couple to Gi, Gz and Go are expected to inhibit the formation of cAMP making assays based upon these types of GPCRs challenging (i.e., the cAMP signal decreases upon activation thus making the direct identification of, e.g, inverse agonists (which would further decrease this signal), interesting. As will be disclosed herein, we have ascertained that for these types of receptors, it is possible to create a GPCR Fusion Protein that is not based upon the endogenous GPCR's endogenous G protein, in an effort to establish a viable cyclase-based assay. Thus, for example, an endogenous Gi coupled receptor can be fused to a Gs protein—we believe that such a fusion construct, upon expression, “drives” or “forces” the endogenous GPCR to couple with, e.g., Gs rather than the “natural” Gi protein, such that a cyclase-based assay can be established. Thus, for Gi, Gz and Go coupled receptors, we prefer that that when a GPCR Fusion Protein is used and the assay is based upon detection of adenylyl cyclase activity, that the fusion construct be established with Gs (or an equivalent G protein that stimulates the formation of the enzyme adenylyl cyclase).

Equally effective is a G Protein Fusion construct that utilizes a Gq Protein fused with a Gs, Gi, Gz or Go Protein. A most preferred fusion construct can be accomplished with a Gq Protein wherein the first six (6) amino acids of the G-protein α-subunit (“Gαq”) is deleted and the last five (5) amino acids at the C-terminal end of Gαq is replaced with the corresponding amino acids of the Gα of the G protein of interest. For example, a fusion construct can have a Gq (6 amino acid deletion) fused with a Gi Protein, resulting in a “Gq/Gi Fusion Construct”. We believe that this fusion construct will force the endogenous Gi coupled receptor to couple to its non-endogenous G protein, Gq, such that the second messenger, for example, inositol triphosphate or diacylgycerol, can be measured in lieu of cAMP production.

4. Co-Transfection of a Target Gi Coupled GPCR with a Signal-Enhancer Gs Coupled GPCR (cAMP Based Assays)

A Gi coupled receptor is known to inhibit adenylyl cyclase, and, therefore, decrease the level of cAMP production, which can make assessment of cAMP levels challenging. An effective technique in measuring the decrease in production of cAMP as an indication of constitutive activation of a receptor that predominantly couples Gi upon activation can be accomplished by co-transfecting a signal enhancer, e.g., a non-endogenous, constitutively activated receptor that predominantly couples with Gs upon activation (e.g., TSHR-A623I, disclosed below), with the Gi linked GPCR. As is apparent, constitutive activation of a Gs coupled receptor can be determined based upon an increase in production of cAMP. Constitutive activation of a Gi coupled receptor leads to a decrease in production cAMP. Thus, the co-transfection approach is intended to advantageously exploit these “opposite” affects. For example, co-transfection of a non-endogenous, constitutively activated Gs coupled receptor (the “signal enhancer”) with the endogenous Gi coupled receptor (the “target receptor”) provides a baseline cAMP signal (i.e., although the Gi coupled receptor will decrease cAMP levels, this “decrease” will be relative to the substantial increase in cAMP levels established by constitutively activated Gs coupled signal enhancer). By then co-transfecting the signal enhancer with a constitutively activated version of the target receptor, cAMP would be expected to further decrease (relative to base line) due to the increased functional activity of the Gi target (i.e., which decreases cAMP).

Screening of candidate compounds using a cAMP based assay can then be accomplished, with two provisos: first, relative to the Gi coupled target receptor, “opposite” effects will result, i.e., an inverse agonist of the Gi coupled target receptor will increase the measured cAMP signal, while an agonist of the Gi coupled target receptor will decrease this signal; second, as would be apparent, candidate compounds that are directly identified using this approach should be assessed independently to ensure that these do not target the signal enhancing receptor (this can be done prior to or after screening against the co-transfected receptors).

### F. Medicinal Chemistry

Generally, but not always, direct identification of candidate compounds is preferably conducted in conjunction with compounds generated via combinatorial chemistry techniques, whereby thousands of compounds are randomly prepared for such analysis. Generally, the results of such screening will be compounds having unique core structures; thereafter, these compounds are preferably subjected to additional chemical modification around a preferred core structure(s) to further enhance the medicinal properties thereof. Such techniques are known to those in the art and will not be addressed in detail in this patent document.

### G. Pharmaceutical Compositions

Candidate compounds selected for further development can be formulated into pharmaceutical compositions using techniques well known to those in the art. Suitable pharmaceutically-acceptable carriers are available to those in the art; for example, see Remington's Pharmaceutical Sciences, 16^{th} Edition, 1980, Mack Publishing Co., (Oslo et al., eds.).

### H. Other Utility

Although a preferred use of the non-endogenous versions the human GPCRs disclosed herein may be for the direct identification of candidate compounds as inverse agonists, agonists or partial agonists (preferably for use as pharmaceutical agents), these versions of human GPCRs can also be utilized in research settings. For example, in vitro and in vivo systems incorporating GPCRs can be utilized to further elucidate and understand the roles these receptors play in the human condition, both normal and diseased, as well as understanding the role of constitutive activation as it applies to understanding the signaling cascade. The value in non-endogenous human GPCRs is that their utility as a research tool is enhanced in that, because of their unique features, non-endogenous human GPCRs can be used to understand the role of these receptors in the human body before the endogenous ligand therefore is identified. Other uses of the disclosed receptors will become apparent to those in the art based upon, inter alia, a review of this patent document.

### EXAMPLES

The following examples are presented for purposes of elucidation, and not limitation, of the present invention. While specific nucleic acid and amino acid sequences are disclosed herein, those of ordinary skill in the art are credited with the ability to make minor modifications to these sequences while achieving the same or substantially similar results reported below. The traditional approach to application or understanding of sequence cassettes from one sequence to another (e.g. from rat receptor to human receptor or from human receptor A to human receptor B) is generally predicated upon sequence alignment techniques whereby the sequences are aligned in an effort to determine areas of commonality. The mutational approach disclosed herein does not rely upon this approach but is instead based upon an algorithmic approach and a positional distance from a conserved proline residue located within the TM6 region of human GPCRs. Once this approach is secured, those in the art are credited with the ability to make minor modifications thereto to achieve substantially the same results (i.e., constitutive activation) disclosed herein. Such modified approaches are considered within the purview of this disclosure.

### Example 1

### Endogenous Human GPCRs

1. Identification of Human GPCRs

The disclosed endogenous human GPCRs were identified based upon a review of the GenBank™ database information. While searching the database, the following cDNA clones were identified as evidenced below (Table C).

TABLE C

| Disclosed | | Complete | Open | Nucleic | Amino |
|---|---|---|---|---|---|
| Human | Accession | DNA | Reading | Acid | Acid |
| Orphan | Number | Sequence | Frame | SEQ. | SEQ. |
| GPCRs | Identified | (Base Pairs) | (Base Pairs) | ID. NO. | ID. NO. |
| hRUP8 | AL121755 | 147,566 bp | 1,152 bp | 1 | 2 |
| hRUP9 | AC0113375 | 143,181 bp | 1,260 bp | 3 | 4 |
| hRUP10 | AC008745 | 94,194 bp | 1,014 bp | 5 | 6 |
| hRUP11 | AC013396 | 155,086 bp | 1,272 bp | 7 | 8 |
| hRUP12 | AP000808 | 177,764 bp | 966 bp | 9 | 10 |
| hRUP13 | AC011780 | 167,819 bp | 1,356 bp | 11 | 12 |
| hRUP14 | AL137118 | 168,297 bp | 1,041 bp | 13 | 14 |
| hRUP15 | AL016468 | 138,828 bp | 1,527 bp | 15 | 16 |
| hRUP16 | AL136106 | 208,042 bp | 1,068 bp | 17 | 18 |
| hRUP17 | AC023078 | 161,735 bp | 969 bp | 19 | 20 |
| hRUP18 | AC008547 | 117,304 bp | 1,305 bp | 21 | 22 |
| hRUP19 | AC026331 | 145,183 bp | 1,041 bp | 23 | 24 |
| hRUP20 | AL161458 | 163,511 bp | 1,011 bp | 25 | 26 |
| hRUP21 | AC026756 | 156,534 bp | 1,014 bp | 27 | 28 |
| hRUP22 | AC027026 | 151,811 bp | 993 bp | 29 | 30 |
| hRUP23 | AC007104 | 200,000 bp | 1,092 bp | 31 | 32 |
| hRUP24 | AL355388 | 190,538 bp | 1,125 bp | 33 | 34 |
| hRUP25 | AC026331 | 145,183 bp | 1,092 bp | 35 | 36 |
| hRUP26 | AC023040 | 178,508 bp | 1,044 bp | 37 | 38 |
| hRUP27 | AC027643 | 158,700 bp | 1,020 bp | 39 | 40 |

2. Full Length Cloning

a. hRUP8 (Seq. Id. Nos. 1 & 2)

The disclosed human RUP8 was identified based upon the use of EST database (dbEST) information. While searching the dbEST, a cDNA clone with accession number AL121755 was identified to encode a novel GPCR. The following PCR primers were used for RT-PCR with human testis Marathon-Ready cDNA (Clontech) as templates:

(SEQ. ID. NO.: 41; sense)
5′-CTTGCAGACATCACCATGGCAGCC-3′
and
(SEQ. ID. NO.: 42; antisense)
5′-GTGATGCTCTGAGTACTGGACTGG-3′.

PCR was performed using Advantage cDNA polymerase (Clontech; manufacturing instructions will be followed) in 50 ul reaction by the following cycles: 94° C. for 30 sec; 94° C. for 10 sec; 65° C. for 20 sec, 72° C. for 1.5 min, and 72° C. for 7 min. Cycles 2 through 4 were repeated 35 times.

A 1.2 kb PCR fragment was isolated and cloned into the pCRII-TOPO vector (Invitrogen) and sequenced using the ABI Big Dye Terminator kit (P.E. Biosystem). See, SEQ.ID.NO.:1. The putative amino acid sequence for RUP8 is set forth in SEQ.ID.NO.:2.

b. hRUP9 (Seq. Id. Nos. 3 & 4)

The disclosed human RUP9 was identified based upon the use of GeneBank database information. While searching the database, a cDNA clone with Accession Number AC011375 was identified as a human genomic sequence from chromosome 5. The full length RUP9 was cloned by PCR using primers:

(SEQ. ID. NO.: 43; sense)
5′-GAAGCTGTGAAGAGTGATGC-3′,
(SEQ. ID. NO.: 44; antisense)
5′-GTCAGCAATATTGATAAGCAGCAG-3′

and human genomic DNA (Promega) as a template. Taq Plus Precision polymerase (Stratagene) was used for the amplification in a 100 μl reaction with 5% DMSO by the following cycle with step 2 to step 4 repeated 35 times: 94° C. for 1 minute; 94° C. for 30 seconds; 56° C. for 30 seconds; 72° C. for 2 minutes; 72° C. for 5 minutes.

A 1.3 Kb PCR fragment was isolated and cloned into the pCRII-TOPO vector (Invitrogen) from 1% agarose gel and completely sequenced using the ABI Big Dye Terminator kit (P.E. Biosystem). See, SEQ.ID.NO.:3. The putative amino acid sequence for RUP8 is set forth in SEQ.ID.NO.:4. The sequence of RUP9 clones isolated from human genomic DNA matched with the sequence obtained from data base.

c. hRUP10 (Seq. Id. Nos. 5 & 6)

The disclosed human RUP10 was identified based upon the use of GenBank database information. While searching the database, a cDNA clone with accession number AC008754 was identified as a human genomic sequence from chromosome 19. The full length RUP10 was cloned by RT-PCR using primers:

(SEQ. ID. NO.: 45; sense)
5′-CCATGGGGAACGATTCTGTCAGCTACG-3′
and
(SEQ. ID. NO.: 46; antisense)
5′-GCTATGCCTGAAGCCAGTCTTGTG-3′

and human leukocyte Marathon-Ready cDNA (Clontech) as a template. Advantage cDNA polymerase (Clontech) was used for the amplification in a 50 μl reaction by the following cycle with step 2 to step 4 repeated 35 times: 94° C. for 30 seconds; 94° C. for 10 seconds; 62° C. for 20 seconds; 72° C. for 1.5 minutes; 72° C. for 7 minutes. A 1.0 Kb PCR fragment was isolated and cloned into the pCRII-TOPO vector (Invitrogen) and completely sequenced using the ABI Big Dye Terminator kit (P.E. Biosystem). The nucleic acid sequence of the novel human receptor RUP10 is set forth in SEQ.ID.NO.:5 and the putative amino acid sequence thereof is set forth in SEQ.ID.NO.:6.

d. hRUP11 (Seq. Id. Nos. 7 & 8)

The disclosed human RUP11 was identified based upon the use of GenBank database information. While searching the database, a cDNA clone with accession number AC013396 was identified as a human genomic sequence from chromosome 2. The full length RUP11 was cloned by PCR using primers:

(SEQ. ID. NO.: 47; sense)
5′-CCAGGATGTTGTGTCACCGTGGTGGC-3′,
(SEQ. ID. NO.: 48; antisense)
5′-CACAGCGCTGCAGCCCTGCAGCTGGC-3′

and human genomic DNA (Clontech) as a template. TaqPlus Precision DNA polymerase (Stratagene) was used for the amplification in a 50 μl reaction by the following cycle with step 2 to step 4 repeated 35 times: 94° C. for 3 minutes; 94° C. for 20 seconds; 67° C. for 20 seconds; 72° C. for 1.5 minutes; 72° C. for 7 minutes. A 1.3 Kb PCR fragment was isolated and cloned into the pCRII-TOPO vector (Invitrogen) and completely sequenced using the ABI Big Dye Terminator kit (P.E. Biosystem). The nucleic acid sequence of the novel human receptor RUP11 is set forth in SEQ.ID.NO.:7 and the putative amino acid sequence thereof is set forth in SEQ.ID.NO.:8.

e. hRUP12 (Seq. Id. Nos. 9 & 10)

The disclosed human RUP12 was identified based upon the use of GenBank database. While searching the database, a cDNA clone with accession number AP000808 was identified to encode a new GPCR, having significant homology with rat RTA and human mas1 oncogene GPCRs. The full length RUP12 was cloned by PCR using primers:

(SEQ. ID. NO.: 49; sense)
5′-CTTCCTCTCGTAGGGATGAACCAGAC-3′
(SEQ. ID. NO.: 50; antisense)
5′-CTCGCACAGGTGGGAAGCACCTGTGG-3′

and human genomic DNA (Clontech) as template. TaqPlus Precision DNA polymerase (Stratagene) was used for the amplification by the following cycle with step 2 to step 4 repeated 35 times: 94° C. for 3 min; 94° C. for 20 sec; 65° C. for 20 sec; 72° C. for 2 min and 72° C. for 7 min. A 1.0 kb PCR fragment was isolated and cloned into the pCRII-TOPO vector (Invitrogen) and completely sequenced using the ABI Big Dye Terminator kit (P.E. Biosystem) (see, SEQ.ID.NO.:9 for nucleic acid sequence and SEQ.ID.NO.:10 for deduced amino acid sequence).

f. hRUP13 (Seq. Id. Nos. 11 & 12)

The disclosed human RUP13 was identified based upon the use of GenBank database. While searching the database, a cDNA clone with accession number AC011780 was identified to encode a new GPCR, having significant homology with GPCR fish GPRX-ORYLA. The full length RUP13 was cloned by PCR using primers:

(SEQ. ID. NO.: 51; sense)
5′-GCCTGTGACAGGAGGTACCCTGG-3′
(SEQ. ID. NO.: 52; antisense)
5′-CATATCCCTCCGAGTGTCCAGCGGC-3′

and human genomic DNA (Clontech) as template. TaqPlus Precision DNA polymerase (Stratagene) was used for the amplification by the following cycle with step 2 to step 4 repeated 35 times: 94° C. for 3 min; 94° C. for 20 sec; 65° C. for 20 sec; 72° C. for 2 min and 72° C. for 7 min. A 1.35 kb PCR fragment was isolated and cloned into the pCRII-TOPO vector (Invitrogen) and completely sequenced using the ABI Big Dye Terminator kit (P.E. Biosystem) (see, SEQ.ID.NO.:11 for nucleic acid sequence and SEQ.ID.NO.:12 for deduced amino acid sequence).

g. hRUP14 (Seq. Id. Nos. 13 & 14)

The disclosed human RUP14 was identified based upon the use of GeneBank database information. While searching the database, a cDNA clone with Accession Number AL137118 was identified as a human genomic sequence from chromosome 13. The full length RUP14 was cloned by PCR using primers:

(SEQ. ID. NO.: 53; sense)
5′-GCATGGAGAGAAAATTTATGTCCTTGCAACC-3′
(SEQ. ID. NO.: 54; antisense)
5′-CAAGAACAGGTCTCATCTAAGAGCTCC-3′

and human genomic DNA (Promega) as a template. Taq Plus Precision polymerase (Stratagene) and 5% DMSO were used for the amplification by the following cycle with step 2 and step 3 repeated 35 times: 94° C. for 3 minute; 94° C. for 20 seconds; 58° C. for 2 minutes; 72° C. for 10 minutes.

A 1.1 Kb PCR fragment was isolated and cloned into the pCRII-TOPO vector (Invitrogen) and completely sequenced using the ABI Big Dye Terminator kit (P.E. Biosystem) (see, SEQ.ID.NO.:13 for nucleic acid sequence and SEQ.ID.NO.:14 for deduced amino acid sequence). The sequence of RUP14 clones isolated from human genomic DNA matched with the sequence obtained from database.

h. hRUP15 (Seq. Id. Nos. 15 & 16)

The disclosed human RUP15 was identified based upon the use of GeneBank database information. While searching the database, a cDNA clone with Accession Number AC016468 was identified as a human genomic sequence. The full length RUP15 was cloned by PCR using primers:

(SEQ. ID. NO.: 5 5; sense)
5′-GCTGTTGCCATGACGTCCACCTGCAC-3′
(SEQ. ID. NO.: 56; antisense)
5′-GGACAGTTCAAGGTTTGCCTTAGAAC-3′

and human genomic DNA (Promega) as a template. Taq Plus Precision polymerase (Stratagene) was used for the amplification by the following cycle with step 2 to 4 repeated 35 times: 94° C. for 3 minute; 94° C. for 20 seconds; 65° C. for 20 seconds; 72° C. for 2 minutes and 72° C. for 7 minutes.

A 1.5 Kb PCR fragment was isolated and cloned into the pCRII-TOPO vector (Invitrogen) and completely sequenced using the ABI Big Dye Terminator kit (P.E. Biosystem). See, SEQ.ID.NO.:15 for nucleic acid sequence and SEQ.ID.NO.:16 for deduced amino acid sequence. The sequence of RUP15 clones isolated from human genomic DNA matched with the sequence obtained from database.

i. hRUP16 (Seq. Id. Nos. 17 & 18)

The disclosed human RUP16 was identified based upon the use of GeneBank database information. While searching the database, a cDNA clone with Accession Number AL136106 was identified as a human genomic sequence from chromosome 13. The full length RUP16 was cloned by PCR using primers:

(SEQ. ID. NO.: 57; sense, 5′ of initiation codon)
5′-CTTTCGATACTGCTCCTATGCTC-3′,
(SEQ. ID. NO.: 58; antisense, 3′ of stop codon)
5′-GTAGTCCACTGAAAGTCCAGTGATCC-3′

and human skeletal muscle Marathon-Ready cDNA (Clontech) as template. Advantage cDNA polymerase (Clontech) was used for the amplification in a 50 ul reaction by the following cycle with step 2 to 4 repeated 35 times: 94° C. for 30 seconds; 94° C. for 5 seconds; 69° C. for 15 seconds; 72° C. for 1 minute and 72° C. for 5 minutes.

A 1.1 Kb PCR fragment was isolated and cloned into the pCRII-TOPO vector (Invitrogen) and completely sequenced using the T7 sequenase kit (Amsham). See, SEQ.ID.NO.:17 for nucleic acid sequence and SEQ.ID.NO.:18 for deduced amino acid sequence. The sequence of RUP16 clones matched with four unordered segments of AL136106, indicating that the RUP16 cDNA is composed of 4 exons.

j. hRUP17 (Seq. Id. Nos. 19 & 20)

The disclosed human RUP17 was identified based upon the use of GeneBank database information. While searching the database, a cDNA clone with Accession Number AC023078 was identified as a human genomic sequence from chromosome 11. The full length RUP17 was cloned by PCR using primers:

(SEQ. ID. NO.: 59; sense, containing initiation codon)
5′-TTTCTGAGCATGGATCCAACCATCTC-3′
(SEQ. ID. NO.: 60; antisense, 3′ of stop codon)
5′-CTGTCTGACAGGGCAGAGGCTCTTC-3′

and human genomic DNA (Promega) as template. Advantage cDNA polymerase mix (Clontech) was used for the amplification in a 100 ul reaction with 5% DMSO by the following cycle with step 2 to 4 repeated 30 times: 94° C. for 1 min; 94° C. for 15 sec; 67° C. for 20 sec; 72° C. for 1 min and 30 sec; and 72° C. for 5 min.

A 970 bp PCR fragment was isolated from 1% agarose gel and cloned into the pCRII-TOPO vector (Invitrogen) and completely sequenced using the ABI Big Dye Termiantor Kit (P.E. Biosystem). See, SEQ.ID.NO.:19 for nucleic acid sequence and SEQ.ID.NO.:20 for deduced amino acid sequence.

k. hRUP18 (Seq. Id. Nos. 21 & 22)

The disclosed human RUP18 was identified based upon the use of GeneBank database information. While searching the database, a cDNA clone with Accession Number AC008547 was identified as a human genomic sequence from chromosome 5. The full length RUP18 was cloned by PCR using primers:

(SEQ.ID.NO.: 61; sense, 5′ of the
initiation codon)
5′-GGAACTCGTATAGACCCAGCGTCGCTCC-3′,
(SEQ.ID.NO.: 62; antisense, 3′ of stop
codon)
5′-GGAGGTTGCGCCTTAGCGACAGATGACC-3′

and human genomic DNA (Promega) as template. TaqPlus precision DNA polymerase (Stratagene) was used for the amplification in a 100 ul reaction with 5% DMSO by the following cycle with step 2 to 4 repeated 35 times: 95° C. for 5 min; 95° C. for 30 sec; 65° C. for 30 sec; 72° C. for 2 min; and 72° C. for 5 min.

A 1.3 kb PCR fragment was isolated from 1% agarose gel and cloned into the pCRII-TOPO vector (Invitrogen) and completely sequenced using the ABI Big Dye Termiantor Kit (P.E. Biosystem). See, SEQ.ID.NO.:21 for nucleic acid sequence and SEQ.ID.NO.:22 for deduced amino acid sequence.

l. hRUP19 (Seq. Id. Nos. 23 & 24)

The disclosed human RUP19 was identified based upon the use of GeneBank database information. While searching the database, a cDNA clone with Accession Number AC026331 was identified as a human genomic sequence from chromosome 12. The full length RUP19 was cloned by PCR using primers:

(SEQ.ID.NO.: 63; sense, 5′ of
initiation codon)
5′-CTGCACCCGGACACTTGCTCTG-3′,
(SEQ.ID.NO.: 64; antisense,
containing the stop codon)
5′-GTCTGCTTGTTCAGTGCCACTCAAC-3′

and human genomic DNA (Promega) as template. TaqPlus Precision DNA polymerase (Stratagene) was used for the amplification with 5% DMSO by the following cycle with step 2 to 4 repeated 35 times: 94° C. for 1 min; 94° C. for 15 sec; 70° C. for 20 sec; 72° C. for 1 min and 30 sec; and 72° C. for 5 min.

A 1.1 kp PCR fragment was isolated from 1% agarose gel and cloned into the pCRII-TOPO vector (Invitrogen) and completely sequenced using the ABI Big Dye Termiantor Kit (P.E. Biosystem). See, SEQ.ID.NO.:23 for nucleic acid sequence and SEQ.ID.NO.:24 for deduced amino acid sequence.

m. hRUP20 (Seq. Id. Nos. 25 & 26)

The disclosed human RUP20 was identified based upon the use of GeneBank database information. While searching the database, a cDNA clone with Accession Number AL161458 was identified as a human genomic sequence from chromosome 1. The full length RUP20 was cloned by PCR using primers:

(SEQ.ID.NO.: 65; sense, 5′ of
initiation codon)
5′-TATCTGCAATTCTATTCTAGCTCCTG-3′,
(SEQ.ID.NO.: 66; antisense,
3′ of stop codon)
5′-TGTCCCTAATAAAGTCACATGAATGC-3′

and human genomic DNA (Promega) as template. Advantage cDNA polymerase mix (Clonetech) was used for the amplification with 5% DMSO by the following cycle with step 2 to 4 repeated 35 times: 94° C. for 1 min; 94° C. for 15 sec; 60° C. for 20 sec; 72° C. for 1 min and 30 sec; and 72° C. for 5 min.

A 1.0 kp PCR fragment was isolated from 1% agarose gel and cloned into the pCRII-TOPO vector (Invitrogen) and completely sequenced using the ABI Big Dye Termiantor Kit (P.E. Biosystem). See, SEQ.ID.NO.:25 for nucleic acid sequence and SEQ.ID.NO.:26 for deduced amino acid sequence.

n. hRUP21 (Seq. Id. Nos. 27 & 28)

The disclosed human RUP21 was identified based upon the use of GeneBank database information. While searching the database, a cDNA clone with Accession Number AC026756 was identified as a human genomic sequence from chromosome 13. The full length RUP21 was cloned by PCR using primers:

(SEQ.ID.NO.: 67; sense)
5′-GGAGACAACCATGAATGAGCCAC-3′
(SEQ.ID.NO.: 68; antisense)
5′-TATTTCAAGGGTTGTTTGAGTAAC-3′

and human genomic DNA (Promega) as template. Taq Plus Precision polymerase (Stratagene) was used for the amplification in a 100 ul reaction with 5% DMSO by the following cycle with step 2 to 4 repeated 30 times: 94° C. for 1 min; 94° C. for 15 sec; 55° C. for 20 sec; 72° C. for 1 min and 30 sec; and 72° C. for 5 min.

A 1,014 bp PCR fragment was isolated from 1% agarose gel and cloned into the pCRII-TOPO vector (Invitrogen) and completely sequenced using the ABI Big Dye Termiantor Kit (RE. Biosystem). See, SEQ.ID.NO.:27 for nucleic acid sequence and SEQ.ID.NO.:28 for deduced amino acid sequence.

o. hRUP22 (Seq. Id. Nos. 29 & 30)

The disclosed human RUP22 was identified based upon the use of GeneBank database information. While searching the database, a cDNA clone with Accession Number AC027026 was identified as a human genomic sequence from chromosome 11. The full length RUP22 was cloned by PCR using primers:

(SEQ.ID.NO.: 69; sense, containing
initiation codon)
5′-GGCACCAGTGGAGGTTTTCTGAGCATG-3′
(SEQ.ID.NO.: 70; antisense, 3′ of
stop codon)
5′-CTGATGGAAGTAGAGGCTGTCCATCTC-3′

and human genomic DNA (Promega) as template. TaqPlus Precision DNA polymerase (Stratagene) was used for the amplification in a 100 ul reaction with 5% DMSO by the following cycle with step 2 to 4 repeated 30 times: 94° C., 1 minutes 94° C., 15 seconds 55° C., 20 seconds 72° C., 1.5 minute 72° C., 5 minutes.

A 970 bp PCR fragment was isolated from 1% agarose gel and cloned into the pCRII-TOPO vector (Invitrogen) and completely sequenced using the ABI Big Dye Termiantor Kit (P.E. Biosystem). See, SEQ.ID.NO.:29 for nucleic acid sequence and SEQ.ID.NO.:30 for deduced amino acid sequence.

p. hRUP23 (Seq. Id. Nos. 31 & 32)

The disclosed human RUP23 was identified based upon the use of GeneBank database information. While searching the database, a cDNA clone with Accession Number AC007104 was identified as a human genomic sequence from chromosome 4. The full length RUP23 was cloned by PCR using primers:

(SEQ.ID.NO.: 71; sense, ATG as
the initiation codon)
5′-CCTGGCGAGCCGCTAGCGCCATG-3′,
(SEQ.ID.NO.: 72; antisense,
TCA as the stop codon)
5′-ATGAGCCCTGCCAGGCCCTCAGT-3′

and human placenta Marathon-Ready cDNA (Clontech) as template. Advantage cDNA polymerase (Clontech) was used for the amplification in a 50 ul reaction by the following cycle with step 2 to 4 repeated 35 times: 95° C. for 30 sec; 95° C. for 15 sec; 66° C. for 20 sec; 72° C. for 1 min and 20 sec; and 72° C. for 5 min.

A 1.0 kb PCR fragment was isolated and cloned into the pCRII-TOPO vector (Invitrogen) and completely sequenced using the ABI Big Dye Terminator Kit (P.E. Biosystem). See, SEQ.ID.NO.:31 for nucleic acid sequence and SEQ.ID.NO.:32 for deduced amino acid sequence.

q. hRUP24 (Seq. Id. Nos. 33 & 34),

The disclosed human RUP25 was identified based upon the use of GeneBank database information. While searching the database, a cDNA clone with Accession Number AC026331 was identified as a human genomic sequence from chromosome 12. The full length RUP25 was cloned by PCR using primers:

(SEQ.ID.NO.: 73; sense,
5′ of initiation codon)
5′-GCTGGAGCATTCACTAGGCGAG-3′,
(SEQ.ID.NO.: 74; antisense,
3′ of stop codon)
5′-AGATCCTGGTTCTTGGTGACAATG-3′

and human genomic DNA (Promega) as template. Advantage cDNA polymerase mix (Clontech) was used for the amplification with 5% DMSO by the following cycle with step 2 to 4 repeated 35 times: 94° C. for 1 minute; 94° C. for 15 seconds; 56° C. for 20 seconds 72° C. for 1 minute 30 seconds and 72° C. for 5 minutes.

A 1.2 kb PCR fragment was isolated from 1% agarose gel and cloned into the pCRII-TOPO vector (Invitrogen) and completely sequenced using the ABI Big Dye Termiantor Kit (RE. Biosystem). See, SEQ.ID.NO.:33 for nucleic acid sequence and SEQ.ID.NO.:34 for deduced amino acid sequence.

r. hRUP25 (Seq. Id. Nos. 35 & 36)

The disclosed human RUP25 was identified based upon the use of GeneBank database information. While searching the database, a cDNA clone with Accession Number AC026331 was identified as a human genomic sequence from chromosome 12. The full length RUP25 was cloned by PCR using primers:

(SEQ.ID.NO.: 75; sense,
5′ of initiation codon)
5′-GCTGGAGCATTCACTAGGCGAG-3′,
(SEQ.ID.NO.: 76; antisense,
3′ of stop codon)
5′-AGATCCTGGTTCTTGGTGACAATG-3′

and human genomic DNA (Promega) as template. Advantage cDNA polymerase mix (Clontech) was used for the amplification with 5% DMSO by the following cycle with step 2 to 4 repeated 35 times: 94° C. for 1 minute; 94° C. for 15 seconds; 56° C. for 20 seconds 72° C. for 1 minute 30 seconds and 72° C. for 5 minutes.

A 1.2 kb PCR fragment was isolated from 1% agarose gel and cloned into the pCRII-TOPO vector (Invitrogen) and completely sequenced using the ABI Big Dye Termiantor Kit (P.E. Biosystem). See, SEQ.ID.NO.:35 for nucleic acid sequence and SEQ.ID.NO.:36 for deduced amino acid sequence.

s. hRUP26 (Seq. Id. Nos. 37 & 38)

The disclosed human RUP26 was identified based upon the use of GeneBank database information. While searching the database, a cDNA clone with Accession Number AC023040 was identified as a human genomic sequence from chromosome 2. The full length RUP26 was cloned by RT-PCR using RUP26 specific primers:

(SEQ.ID.NO.: 77; sense,
containing initiation codon)
5′-AGCCATCCCTGCCAGGAAGCATGG-3′
(SEQ.ID.NO.: 78; antisense,
containing stop codon)
5′-CCAGACTGTGGACTCAAGAACTCTAGG-3′

and human pancreas Marathon—Ready cDNA (Clontech) as template. Advantage cDNA polymerase mix (Clontech) was used for the amplification in a 100 μl reaction with 5% DMSO by the following cycle with step 2 to 4 repeated 35 times: 94° C. for 5 minute; 95° C. for 30 seconds; 65° C. for 30 seconds 72° C. for 2 minute and 72° C. for 5 minutes.

A 1.1 kb PCR fragment was isolated from 1% agarose gel and cloned into the pCRII-TOPO vector (Invitrogen) and completely sequenced using the ABI Big Dye Termiantor Kit (P.E. Biosystem). See, SEQ.ID.NO.:37 for nucleic acid sequence and SEQ.ID.NO.:38 for deduced amino acid sequence.

t. hRUP27 (Seq. Id. Nos. 39 & 40)

The disclosed human RUP27 was identified based upon the use of GeneBank database information. While searching the database, a cDNA clone with Accession Number AC027643 was identified as a human genomic sequence from chromosome 12. The full length RUP27 was cloned by PCR using RUP27 specific primers:

(SEQ.ID.NO.: 79; sense, containing
initiation codon)
5′-AGTCCACGAACAATGAATCCATTTCATG-3′,
(SEQ.ID.NO.: 80; antisense,
3′ of stop codon)
5′-ATCATGTCTAGACTCATGGTGATCC-3′

and the human adult brain Marathon-Ready cDNA (Clontech) as template. Advantage cDNA polymerase mix (Clontech) was used for the amplification in a 50 μl reaction with 5% DMSO by the following cycle with step 2 to 4 repeated 35 times: 94° C. for 1 minute; 94° C. for 10 seconds; 58° C. for 20 seconds 72° C. for 1 minute 30 seconds and 72° C. for 5 minutes.

A 1.1 kb PCR fragment was isolated from 1% agarose gel and cloned into the pCRII-TOPO vector (Invitrogen) and completely sequenced using the ABI Big Dye Termiantor Kit (P.E. Biosystem). See, SEQ.ID.NO.:35 for nucleic acid sequence and SEQ.ID.NO.:36 for deduced amino acid sequence. The sequence of RUP27 cDNA clone isolated from human brain was determined to match with five unordered segments of AC027643, indicating that the RUP27 cDNA is composed of 5 exons.

### Example 2

### Preparation of Non-Endogenous, Constitutively Activated GPCRs

Those skilled in the art are credited with the ability to select techniques for mutation of a nucleic acid sequence. Presented below are approaches utilized to create non-endogenous versions of several of the human GPCRs disclosed above. The mutations disclosed below are based upon an algorithmic approach whereby the 16^{th} amino acid (located in the IC3 region of the GPCR) from a conserved proline (or an endogenous, conservative substitution therefore) residue (located in the TM6 region of the GPCR, near the TM6/IC3 interface) is mutated, preferably to an alanine, histidine, arginine or lysine amino acid residue, most preferably to a lysine amino acid residue.

1. Transformer Site-Directed™ Mutagenesis

Preparation of non-endogenous human GPCRs may be accomplished on human GPCRs using Transformer Site-Directed™ Mutagenesis Kit (Clontech) according to the manufacturer instructions. Two mutagenesis primers are utilized, most preferably a lysine mutagenesis oligonucleotide that creates the lysine mutation, and a selection marker oligonucleotide. For convenience, the codon mutation to be incorporated into the human GPCR is also noted, in standard form (Table D):

TABLE D

| Receptor Identifier | Codon Mutation |
|---|---|
| hRUP8 | V274K |
| hRUP9 | T249K |
| hRUP10 | R232K |
| hRUP11 | M294K |
| hRUP12 | F220K |
| hRUP16 | A238K |
| hRUP17 | Y215K |
| hRUP18 | L294K |
| hRUP19 | T219K |
| hRUP20 | K248A |
| | K248H |
| | K248R |
| hRUP21 | R240K |
| hRUP22 | Y222K |
| hRUP24 | A245K |
| hRUP25 | I230K |
| hRUP26 | V285K |
| hRUP27 | T248K |

2. QuikChange™ Site-Directed™ Mutagenesis

Preparation of non-endogenous human GPCRs can also be accomplished by using QuikChange™ Site-Directed™ Mutagenesis Kit (Stratagene, according to manufacturer's instructions). Endogenous GPCR is preferably used as a template and two mutagenesis primers utilized, as well as, most preferably, a lysine mutagenesis oligonucleotide and a selection marker oligonucleotide (included in kit). For convenience, the codon mutation incorporated into the novel human GPCR and the respective oligonucleotides are noted, in standard form (Table E):

TABLE E

| | | 5′-3′ orientation | | |
|---|---|---|---|---|
| | | (sense), | 5′-3′ | Cycle Conditions |
| | | (SEQ.ID.NO.) | orientation | Min (′), Sec (°) |
| Receptor | Codon | mutation | (antisense) | Cycles 2-4 |
| Identifier | Mutation | underlined | (SEQ.ID.NO.) | repeated 16 times |
| hRUP13 | A268K | GGGGAGGGAAAGCAA | CCAGGAGAACCACCT | 98° for 2′ |
| | | AGGTGGTCCTCCTGG | TTGCTTTCCCTCCCC | 98° for 30″ |
| | | (81) | (82) | 56° C. for 30″ |
| | | | | 72° for 11′ 40″ |
| | | | | 72° for 5′ |
| hRUP14 | L246K | CAGGAAGGCAAAGAC | GATGATGATGGTGGT | 98° for 2′ |
| | | CACCATCATCATC | CTTTGCCTTCCTG | 98° for 30″ |
| | | (85) | (86) | 55° C. for 30″ |
| | | | | 72° for 11′ 40″ |
| | | | | 72° for 5′ |
| hRUP15 | A398K | CCAGTGCAAAGCTAAG | GAAGATCACTTTCTTA | 98° for 2′ |
| | | AAAGTGATCTTC | GCTTTGCACTGG | 98° for 30″ |
| | | (89) | (90) | 55° C. for 30″ |
| | | | | 72° for 11′ 40″ |
| | | | | 72° for 5′ |
| hRUP23 | W275K | GCCGCCACCGCGCCAA | GCCAATCTTCCTCTTG | 98° for 2′ |
| | | GAGGAAGATTGGC | GCGCGGTGGCGGC | 98° for 30″ |
| | | (93) | (94) | 56° C. for 30″ |
| | | | | 72° for 11′ 40″ |
| | | | | 72° for 5′ |

The non-endogenous human GPCRs were then sequenced and the derived and verified nucleic acid and amino acid sequences are listed in the accompanying “Sequence Listing” appendix to this patent document, as summarized in Table F below:

TABLE F

| Non Endogenous | Nucleic Acid Sequence | Amino Acid Sequence |
|---|---|---|
| Human GPCR | Listing | Listing |
| hRUP13 | SEQ. ID. NO.: 83 | SEQ. ID. NO.: 84 |
| hRUP14 | SEQ. ID. NO.: 87 | SEQ. ID. NO.: 88 |
| hRUP15 | SEQ. ID. NO.: 91 | SEQ. ID. NO.: 92 |
| hRUP23 | SEQ. ID. NO.: 95 | SEQ. ID. NO.: 96 |

### Example 3

### Receptor Expression

Although a variety of cells are available to the art for the expression of proteins, it is most preferred that mammalian cells be utilized. The primary reason for this is predicated upon practicalities, i.e., utilization of, e.g., yeast cells for the expression of a GPCR, while possible, introduces into the protocol a non-mammalian cell which may not (indeed, in the case of yeast, does not) include the receptor-coupling, genetic-mechanism and secretary pathways that have evolved for mammalian systems—thus, results obtained in non-mammalian cells, while of potential use, are not as preferred as that obtained from mammalian cells. Of the mammalian cells, COS-7, 293 and 293T cells are particularly preferred, although the specific mammalian cell utilized can be predicated upon the particular needs of the artisan.

a. Transient Transfection

On day one, 6×10⁶/10 cm dish of 293 cells well were plated out. On day two, two reaction tubes were prepared (the proportions to follow for each tube are per plate): tube A was prepared by mixing 4 μg DNA (e.g., pCMV vector; pCMV vector with receptor cDNA, etc.) in 0.5 ml serum free DMEM (Gibco BRL); tube B was prepared by mixing 24 μl lipofectamine (Gibco BRL) in 0.5 ml serum free DMEM. Tubes A and B were admixed by inversions (several times), followed by incubation at room temperature for 30-45 min. The admixture is referred to as the “transfection mixture”. Plated 293 cells were washed with 1×PBS, followed by addition of 5 ml serum free DMEM. 1 ml of the transfection mixture were added to the cells, followed by incubation for 4 hrs at 37° C./5% CO₂. The transfection mixture was removed by aspiration, followed by the addition of 10 ml of DMEM/10% Fetal Bovine Serum. Cells were incubated at 37° C./5% CO₂. After 48 hr incubation, cells were harvested and utilized for analysis.

b. Stable Cell Lines: Gs Fusion Protein

Approximately 12×10⁶ 293 cells are plated on a 15 cm tissue culture plate. Grown in DME High Glucose Medium containing ten percent fetal bovine serum and one percent sodium pyruvate, L-glutamine, and anti-biotics. Twenty-four hours following plating of 293 cells to ˜80% confluency, the cells are transfected using 12 μg of DNA. The 12 μg of DNA is combined with 60 ul of lipofectamine and 2 mL of DME High Glucose Medium without serum. The medium is aspirated from the plates and the cells are washed once with medium without serum. The DNA, lipofectamine, and medium mixture is added to the plate along with 10 mL of medium without serum. Following incubation at 37 degrees Celsius for four to five hours, the medium is aspirated and 25 ml of medium containing serum is added. Twenty-four hours following transfection, the medium is aspirated again, and fresh medium with serum is added. Forty-eight hours following transfection, the medium is aspirated and medium with serum is added containing geneticin (G418 drug) at a final concentration of 500 μg/mL. The transfected cells now undergo selection for positively transfected cells containing the G418 resistant gene. The medium is replaced every four to five days as selection occurs. During selection, cells are grown to create stable pools, or split for stable clonal selection.

### Example 4

### Assays for Determination of Constitutive Activity of non-endogenous GPCRs

A variety of approaches are available for assessment of constitutive activity of the non-endogenous human GPCRs. The following are illustrative; those of ordinary skill in the art are credited with the ability to determine those techniques that are preferentially beneficial for the needs of the artisan.

1. Membrane Binding Assays: [³⁵S]GTPγS Assay

When a G protein-coupled receptor is in its active state, either as a result of ligand binding or constitutive activation, the receptor couples to a G protein and stimulates the release of GDP and subsequent binding of GTP to the G protein. The alpha subunit of the G protein-receptor complex acts as a GTPase and slowly hydrolyzes the GTP to GDP, at which point the receptor normally is deactivated. Constitutively activated receptors continue to exchange GDP for GTP. The non-hydrolyzable GTP analog, [³⁵S]GTPγS, can be utilized to demonstrate enhanced binding of [³⁵S]GTPγS to membranes expressing constitutively activated receptors. The advantage of using [³⁵S]GTPγS binding to measure constitutive activation is that: (a) it is generically applicable to all G protein-coupled receptors; (b) it is proximal at the membrane surface making it less likely to pick-up molecules which affect the intracellular cascade.

The assay utilizes the ability of G protein coupled receptors to stimulate [³⁵S]GTPγS binding to membranes expressing the relevant receptors. The assay can, therefore, be used in the direct identification method to screen candidate compounds to known, orphan and constitutively activated G protein-coupled receptors. The assay is generic and has application to drug discovery at all G protein-coupled receptors.

The [³⁵S]GTPγS assay was incubated in 20 mM HEPES and between 1 and about 20 mM MgCl₂ (this amount can be adjusted for optimization of results, although 20 mM is preferred) pH 7.4, binding buffer with between about 0.3 and about 1.2 nM [³⁵S]GTPγS (this amount can be adjusted for optimization of results, although 1.2 is preferred) and 12.5 to 75 μg membrane protein (e.g, 293 cells expressing the Gs Fusion Protein; this amount can be adjusted for optimization) and 10 μM GDP (this amount can be changed for optimization) for 1 hour. Wheatgerm agglutinin beads (25 μl; Amersham) were then added and the mixture incubated for another 30 minutes at room temperature. The tubes were then centrifuged at 1500×g for 5 minutes at room temperature and then counted in a scintillation counter.

2. Adenylyl Cyclase

A Flash Plate™ Adenylyl Cyclase kit (New England Nuclear; Cat. No. SMP004A) designed for cell-based assays can be modified for use with crude plasma membranes. The Flash Plate wells can contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells were harvested approximately twenty four hours after transient transfection. Media is carefully aspirated off and discarded. 10 ml of PBS is gently added to each dish of cells followed by careful aspiration. 1 ml of Sigma cell dissociation buffer and 3 ml of PBS are added to each plate. Cells were pipeted off the plate and the cell suspension was collected into a 50 ml conical centrifuge tube. Cells were then centrifuged at room temperature at 1,100 rpm for 5 min. The cell pellet was carefully re-suspended into an appropriate volume of PBS (about 3 ml/plate). The cells were then counted using a hemocytometer and additional PBS was added to give the appropriate number of cells (with a final volume of about 50 μl/well).

cAMP standards and Detection Buffer (comprising 1 μCi of tracer [¹²⁵I cAMP (50 μl] to 11 ml Detection Buffer) was prepared and maintained in accordance with the manufacturer's instructions. Assay Buffer was prepared fresh for screening and contained 50 μl of Stimulation Buffer, 3 ul of test compound (12 uM final assay concentration) and 50 μl cells, Assay Buffer was stored on ice until utilized. The assay was initiated by addition of 500 of cAMP standards to appropriate wells followed by addition of 50 μl of PBSA to wells H-11 and H12. 50 μl of Stimulation Buffer was added to all wells. DMSO (or selected candidate compounds) was added to appropriate wells using a pin tool capable of dispensing 3 μl of compound solution, with a final assay concentration of 12 μM test compound and 100 μl total assay volume. The cells were then added to the wells and incubated for 60 min at room temperature. 100 μl of Detection Mix containing tracer cAMP was then added to the wells. Plates were then incubated additional 2 hours followed by counting in a Wallac MicroBeta scintillation counter. Values of cAMP/well were then extrapolated from a standard cAMP curve which was contained within each assay plate.

3. Cell-Based cAMP for Gi Coupled Target GPCRs

TSHR is a Gs coupled GPCR that causes the accumulation of cAMP upon activation. TSHR will be constitutively activated by mutating amino acid residue 623 (i.e., changing an alanine residue to an isoleucine residue). A Gi coupled receptor is expected to inhibit adenylyl cyclase, and, therefore, decrease the level of cAMP production, which can make assessment of cAMP levels challenging. An effective technique for measuring the decrease in production of cAMP as an indication of constitutive activation of a Gi coupled receptor can be accomplished by co-transfecting, most preferably, non-endogenous, constitutively activated TSHR (TSHR-A623I) (or an endogenous, constitutively active Gs coupled receptor) as a “signal enhancer” with a Gi linked target GPCR to establish a baseline level of cAMP. Upon creating a non-endogenous version of the Gi coupled receptor, this non-endogenous version of the target GPCR is then co-transfected with the signal enhancer, and it is this material that can be used for screening. We will utilize such approach to effectively generate a signal when a cAMP assay is used; this approach is preferably used in the direct identification of candidate compounds against Gi coupled receptors. It is noted that for a Gi coupled GPCR, when this approach is used, an inverse agonist of the target GPCR will increase the cAMP signal and an agonist will decrease the cAMP signal.

On day one, 2×10⁴ 293 and 293 cells/well will be plated out. On day two, two reaction tubes will be prepared (the proportions to follow for each tube are per plate): tube A will be prepared by mixing 2 μg DNA of each receptor transfected into the mammalian cells, for a total of 4 μg DNA (e.g., pCMV vector; pCMV vector with mutated THSR (TSHR-A623I); TSHR-A623I and GPCR, etc.) in 1.2 ml serum free DMEM (Irvine Scientific, Irvine, Calif.); tube B will be prepared by mixing 120 μl lipofectamine (Gibco BRL) in 1.2 ml serum free DMEM. Tubes A and B will then be admixed by inversions (several times), followed by incubation at room temperature for 30-45 min. The admixture is referred to as the “transfection mixture”. Plated 293 cells will be washed with 1×PBS, followed by addition of 10 ml serum free DMEM. 2.4 ml of the transfection mixture will then be added to the cells, followed by incubation for 4 hrs at 37° C./5% CO₂. The transfection mixture will then be removed by aspiration, followed by the addition of 25 ml of DMEM/10% Fetal Bovine Serum. Cells will then be incubated at 37° C./5% CO₂. After 24 hr incubation, cells will then be harvested and utilized for analysis.

A Flash Plate™ Adenylyl Cyclase kit (New England Nuclear; Cat. No. SMP004A) is designed for cell-based assays, however, can be modified for use with crude plasma membranes depending on the need of the skilled artisan. The Flash Plate wells will contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express the receptors.

Transfected cells will be harvested approximately twenty four hours after transient transfection. Media will be carefully aspirated off and discarded. 10 ml of PBS will be gently added to each dish of cells followed by careful aspiration. 1 ml of Sigma cell dissociation buffer and 3 ml of PBS will be added to each plate. Cells will be pipeted off the plate and the cell suspension will be collected into a 50 ml conical centrifuge tube. Cells will then be centrifuged at room temperature at 1,100 rpm for 5 min. The cell pellet will be carefully re-suspended into an appropriate volume of PBS (about 3 ml/plate). The cells will then be counted using a hemocytometer and additional PBS is added to give the appropriate number of cells (with a final volume of about 50 μl/well).

cAMP standards and Detection Buffer (comprising 1 μCi of tracer [¹²⁵I cAMP (50 μl] to 11 ml Detection Buffer) will be prepared and maintained in accordance with the manufacturer's instructions. Assay Buffer should be prepared fresh for screening and contained 50 μl of Stimulation Buffer, 3 ul of test compound (12 uM final assay concentration) and 50 μl cells, Assay Buffer can be stored on ice until utilized. The assay can be initiated by addition of 50 μl of cAMP standards to appropriate wells followed by addition of 50 μl of PBSA to wells H-11 and H12. 50 ul of Stimulation Buffer will be added to all wells. Selected compounds (e.g., TSH) will be added to appropriate wells using a pin tool capable of dispensing 3 μl of compound solution, with a final assay concentration of 12 μM test compound and 100 μl total assay volume. The cells will then be added to the wells and incubated for 60 min at room temperature. 100 μl of Detection Mix containing tracer cAMP will then be added to the wells. Plates were then incubated additional 2 hours followed by counting in a Wallac MicroBeta scintillation counter. Values of cAMP/well will then be extrapolated from a standard cAMP curve which is contained within each assay plate.

4. Reporter-Based Assays

a. CRE-Luc Reporter Assay (Gs-Associated Receptors)

293 and 293T cells are plated-out on 96 well plates at a density of 2×10⁴ cells per well and were transfected using Lipofectamine Reagent (BRL) the following day according to manufacturer instructions. A DNA/lipid mixture is prepared for each E-well transfection as follows: 260 ng of plasmid DNA in 100 μl of DMEM were gently mixed with 2 μl of lipid in 100 μl of DMEM (the 260 ng of plasmid DNA consisted of 200 ng of a 8×CRE-Luc reporter plasmid, 50 ng of pCMV comprising endogenous receptor or non-endogenous receptor or pCMV alone, and 10 ng of a GPRS expression plasmid (GPRS in pcDNA3 (Invitrogen)). The 8×CRE-Luc reporter plasmid was prepared as follows: vector SRIF-β-gal was obtained by cloning the rat somatostatin promoter (−71/+51) at BglV-HindIII site in the pβgal-Basic Vector (Clontech). Eight (8) copies of cAMP response element were obtained by PCR from an adenovirus template AdpCF126CCRE8 (see, 7 Human Gene Therapy 1883 (1996)) and cloned into the SRIF-β-gal vector at the Kpn-BglV site, resulting in the 8×CRE-β-gal reporter vector. The 8×CRE-Luc reporter plasmid was generated by replacing the beta-galactosidase gene in the 8×CRE-β-gal reporter vector with the luciferase gene obtained from the pGL3-basic vector (Promega) at the HindIII-BamHI site. Following 30 min. incubation at room temperature, the DNA/lipid mixture was diluted with 400 μl of DMEM and 1000 of the diluted mixture was added to each well. 100 μl of DMEM with 10% FCS were added to each well after a 4 hr incubation in a cell culture incubator. The following day the transfected cells were changed with 200 μl/well of DMEM with 10% FCS. Eight (8) hours later, the wells were changed to 100 μl/well of DMEM without phenol red, after one wash with PBS. Luciferase activity were measured the next day using the LucLite™ reporter gene assay kit (Packard) following manufacturer instructions and read on a 1450 MicroBeta™ scintillation and luminescence counter (Wallac).

b. AP1 Reporter Assay (Gq-Associated Receptors)

A method to detect Gq stimulation depends on the known property of Gq-dependent phospholipase C to cause the activation of genes containing AP1 elements in their promoter. A Pathdetect™ AP-1 cis-Reporting System (Stratagene, Catalogue #219073) can be utilized following the protocol set forth above with respect to the CREB reporter assay, except that the components of the calcium phosphate precipitate were 410 ng pAP1-Luc, 80 ng pCMV-receptor expression plasmid, and 20 ng CMV-SEAP.

c. SRF-Luc Reporter Assay (Gq-Associated Receptors)

One method to detect Gq stimulation depends on the known property of Gq-dependent phospholipase C to cause the activation of genes containing serum response factors in their promoter. A Pathdetect™ SRF-Luc-Reporting System (Stratagene) can be utilized to assay for Gq coupled activity in, e.g., COS7 cells. Cells are transfected with the plasmid components of the system and the indicated expression plasmid encoding endogenous or non-endogenous GPCR using a Mammalian Transfection™ Kit (Stratagene, Catalogue #200285) according to the manufacturer's instructions. Briefly, 410 ng SRF-Luc, 80 ng pCMV-receptor expression plasmid and 20 ng CMV-SEAP (secreted alkaline phosphatase expression plasmid; alkaline phosphatase activity is measured in the media of transfected cells to control for variations in transfection efficiency between samples) are combined in a calcium phosphate precipitate as per the manufacturer's instructions. Half of the precipitate is equally distributed over 3 wells in a 96-well plate, kept on the cells in a serum free media for 24 hours. The last 5 hours the cells are incubated with 1 Angiotensin, where indicated. Cells are then lysed and assayed for luciferase activity using a Luclite™ Kit (Packard, Cat. #6016911) and “Trilux 1450 Microbeta” liquid scintillation and luminescence counter (Wallac) as per the manufacturer's instructions. The data can be analyzed using GraphPad Prism™ 2.0a (GraphPad Software Inc.).

d. Intracellular IP₃ Accumulation Assay (Gq-Associated Receptors)

On day 1, cells comprising the receptors (endogenous and/or non-endogenous) can be plated onto 24 well plates, usually 1×10⁵ cells/well (although his umber can be optimized. On day 2 cells can be transfected by firstly mixing 0.25 μg DNA in 50 μl serum free DMEM/well and 2 μl lipofectamine in 50 μl serumfree DMEM/well. The solutions are gently mixed and incubated for 15-30 min at room temperature. Cells are washed with 0.5 ml PBS and 400 μl of serum free media is mixed with the transfection media and added to the cells. The cells are then incubated for 3-4 hrs at 37° C./5% CO₂ and then the transfection media is removed and replaced with 1 ml/well of regular growth media. On day 3 the cells are labeled with ³H-myo-inositol. Briefly, the media is removed and the cells are washed with 0.5 ml PBS. Then 0.5 ml inositol-free/serum free media (GIBCO BRL) is added/well with 0.25 μCi of ³H-myo-inositol/well and the cells are incubated for 16-18 hrs o/n at 37° C./5% CO₂. On Day 4 the cells are washed with 0.5 ml PBS and 0.45 ml of assay medium is added containing inositol-free/serum free media 10 μM pargyline 10 mM lithium chloride or 0.4 ml of assay medium and 50 μl of 10× ketanserin (ket) to final concentration of 10 μM. The cells are then incubated for 30 min at 37° C. The cells are then washed with 0.5 ml PBS and 200 μl of fresh/icecold stop solution (1M KOH; 18 mM Na-borate; 3.8 mM EDTA) is added/well. The solution is kept on ice for 5-10 min or until cells were lysed and then neutralized by 200 μl of fresh/ice cold neutralization sol. (7.5% HCL). The lysate is then transferred into 1.5 ml eppendorf tubes and 1 ml of chloroform/methanol (1:2) is added/tube. The solution is vortexed for 15 sec and the upper phase is applied to a Biorad AG1-X8™ anion exchange resin (100-200 mesh). Firstly, the resin is washed with water at 1:1.25 W/V and 0.9 ml of upper phase is loaded onto the column. The column is washed with 10 mls of 5 mM myo-inositol and 10 ml of 5 mM Na-borate/60 mM Na-formate. The inositol tris phosphates are eluted into scintillation vials containing 10 ml of scintillation cocktail with 2 ml of 0.1 M formic acid/1 M ammonium formate. The columns are regenerated by washing with 10 ml of 0.1 M formic acid/3M ammonium formate and rinsed twice with dd H₂O and stored at 4° C. in water.

Exemplary results are presented below in Table G:

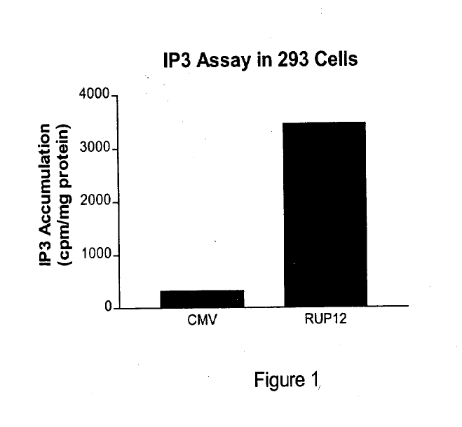

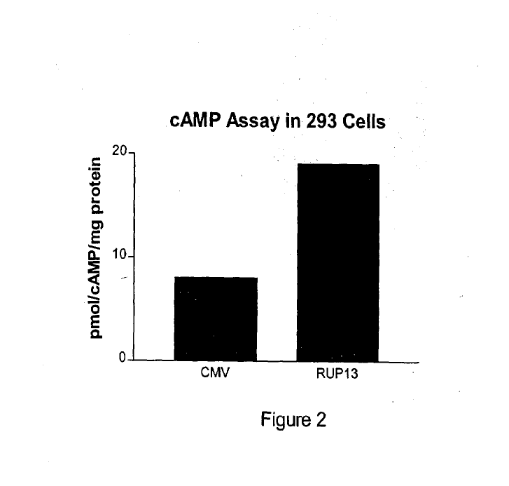

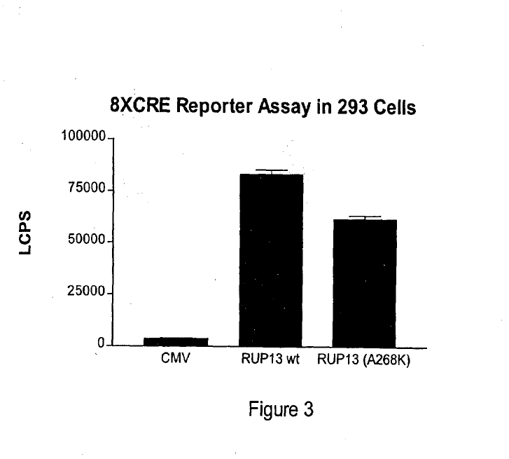

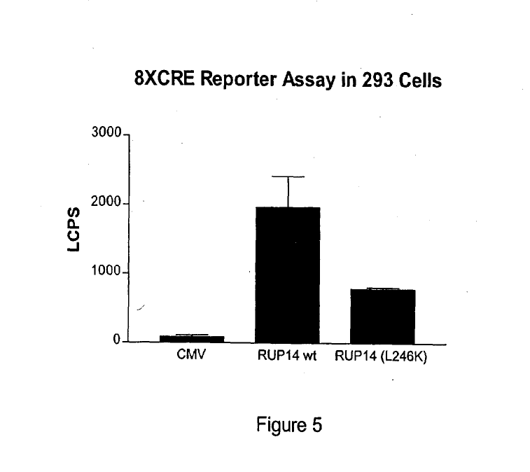

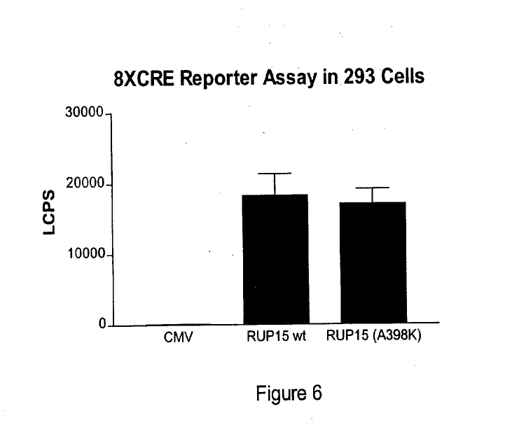

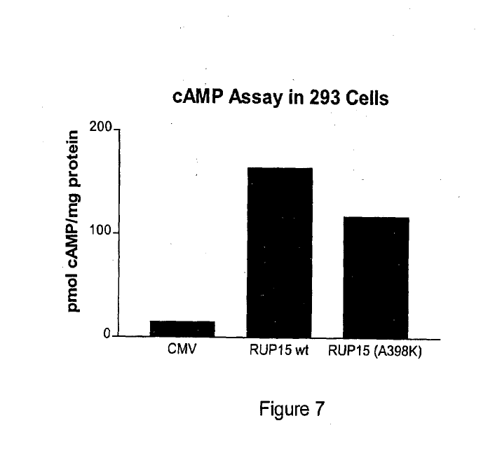

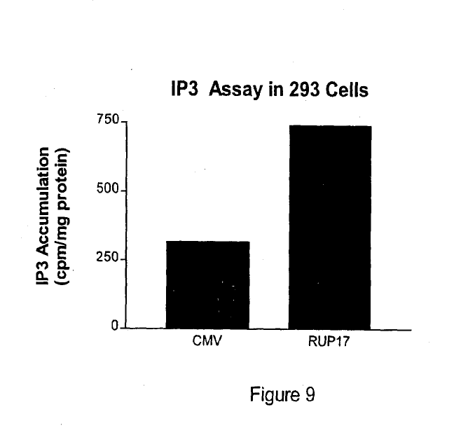

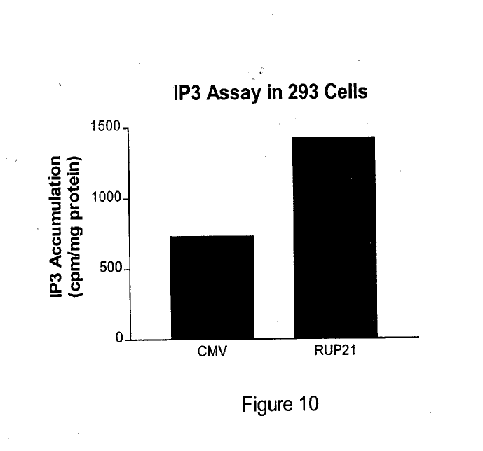

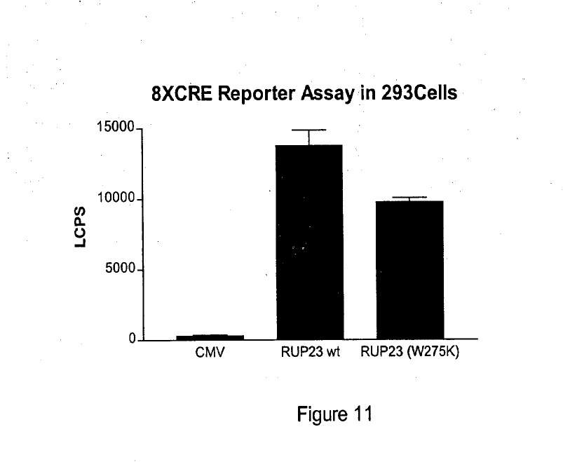

TABLE G

| | | | | Signal | Signal | Difference |
|---|---|---|---|---|---|---|
| | | | | Generated: | Generated: | ( [symbol] <) |
| | | Assay | Signal | Endogenous | Non-Endogenous | Between |
| | | Utilized | Generated: | Version (Relative | Version (Relative | CMV v. Wild-type |
| Receptor | Mutation | Figure No.) | CMV | Light Units) | Light Units) | Wild-type v. Mutant |
| hRUP12 | N/A | IP₃ | 317.03 | 3463.29 | — | 1. 11 Fold [symbol] |
| | | (FIG. 1) | cpm/mg protein | cpm/mg protein | | |
| hRUP13 | N/A | cAMP | 8.06 | 19.10 | — | 1. 2.4 Fold [symbol] |
| | | (FIG. 2) | pmol/cAMP/mg | pmol/cAMP/mg | | |
| | | | protein | protein | | |
| | A268K | 8XCRE- | 3665.43 | 83280.17 | 61713.6 | 1. 23 Fold [symbol] |
| | | LUC | LCPS | LPCS | LCPS | 2. 26% < |
| | | (FIG. 3) | | | | |
| hRUP14 | L246K | 8XCRE- | 86.07 | 1962.87 | 789.73 | 1. 23 Fold [symbol] |
| | | LUC | LCPS | LCPS | LCPS | 2. 60% < |
| | | (FIG. 5) | | | | |
| hRUP15 | A398K | 8XCRE- | 86.07 | 18286.77 | 17034.83 | 1. 212 Fold [symbol] |
| | | LUC | LCPS | LCPS | LCPS | 2. 1% < |
| | | (FIG. 6) | | | | |
| | A398K | cAMP | 15.00 | 164.4 | 117.5 | 1. 11 Fold [symbol] |
| | | (FIG. 7) | pmol/cAMP/mg | pmol/cAMP/mg | pmol/cAMP/ | 2. 29% < |
| | | | protein | protein | mg protein | |
| hRUP17 | N/A | IP₃ | 317.03 | 741.07 | — | 1. 2.3 Fold [symbol] |
| | | (FIG. 9) | cpm/mg protein | cpm/mg protein | | |
| hRUP21 | N/A | IP₃ | 730.5 | 1421.9 | — | 1. 2 Fold [symbol] |
| | | (FIG. 10) | cpm/mg protein | cpm/mg protein | | |
| hRUP23 | W275K | 8XCRE- | 311.73 | 13756.00 | 9756.87 | 1. 44 Fold [symbol] |
| | | LUC | pmol/cAMP/mg | pmol/cAMP/mg | pmol/cAMP/ | 2. 30% < |
| | | (FIG. 11) | protein | protein | mg protein | |

N/A = not applied

Exemplary results of GTPγS assay for detecting constitutive activation, as disclosed in Example 4(1) above, was accomplished utilizing Gs:Fusion Protein Constructs on human RUP13 and RUP15. Table H below lists the signals generated from this assay and the difference in signals as indicated:

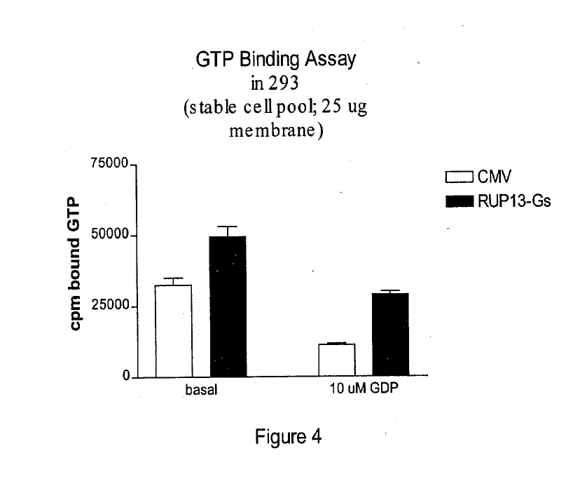

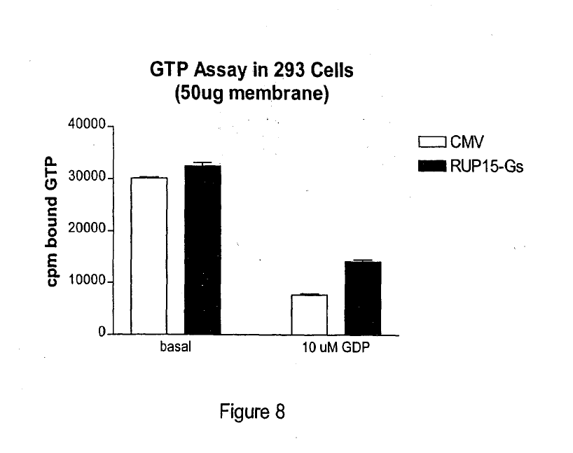

TABLE H

| | | | | | | Difference Between: |
|---|---|---|---|---|---|---|
| | | | Signal | Signal | Signal | 1. CMV v. Fusion Protein |
| | | Signal | Generated: | Generated: | Generated: | 2. CMV + GDP |
| Receptor: | | Generated: | Fusion | CMV + | Fusion Protein + | vs. Fusion + GDP |
| Gs Fusion | Assay | CMV | Protein | 10 μM GDP | 10 μM GDP | 3. Fusion vs. Fusion + GDP |
| Protein | Utilized | (cpm bound GTP) | (cpm bound GTP) | (cpm bound GTP) | (cpm bound GTP) | (cpm bound GTP) |
| hRUP13-Gs | GTPγS | 32494.0 | 49351.30 | 11148.30 | 28834.67 | 1. 1.5 Fold [symbol] |
| | (FIG. 4) | | | | | 2. 2.6 Fold [symbol] |
| | | | | | | 3. 42% < |
| hRUP15-Gs | GTPγS | 30131.67 | 32493.67 | 7697.00 | 14157.33 | 1. 1.1 Fold [symbol] |
| | (FIG. 8) | | | | | 2. 1.8 Fold [symbol] |
| | | | | | | 3. 56% < |

### Example 5

### Fusion Protein Preparation

a. GPCR:Gs Fusion Construct

The design of the constitutively activated GPCR-G protein fusion construct was accomplished as follows: both the 5′ and 3′ ends of the rat G protein Gsα (long form; Itoh, H. et al., 83 PNAS 3776 (1986)) were engineered to include a HindIII (5′-AAGCTT-3′) sequence thereon. Following confirmation of the correct sequence (including the flanking HindIII sequences), the entire sequence was shuttled into pcDNA3.1(−) (Invitrogen, cat. no. V795-20) by subcloning using the HindIII restriction site of that vector. The correct orientation for the Gsα sequence was determined after subcloning into pcDNA3.1(−). The modified pcDNA3.1(−) containing the rat Gsα gene at Hindu sequence was then verified; this vector was now available as a “universal” Gsα protein vector. The pcDNA3.1(−) vector contains a variety of well-known restriction sites upstream of the HindIII site, thus beneficially providing the ability to insert, upstream of the Gs protein, the coding sequence of an endogenous, constitutively active GPCR. This same approach can be utilized to create other “universal” G protein vectors, and, of course, other commercially available or proprietary vectors known to the artisan can be utilized—the important criteria is that the sequence for the GPCR be upstream and in-frame with that of the G protein.

RUP13 couples via Gs. For the following exemplary GPCR Fusion Proteins, fusion to Gsα was accomplished.

A RUP13-Gsα Fusion Protein construct was made as follows: primers were designed as follows:

(SEQ.ID.NO.: 97; sense)
5′-gatc[TCTAGAAT]GGAGTCCTCACCCATCCCCCAG -3′
(SEQ.ID.NO.: 98; antisense)
5′-gatc[GATATC]CGTGACTCCAGCCGGGGTGAGGCGGC-3′.

Nucleotides in lower caps are included as spacers in the restriction sites (designated in brackets) between the G protein and RUP13. The sense and anti-sense primers included the restriction sites for XbaI and EcoRV, respectively, such that spacers (attributed to the restriction sites) exists between the G protein and RUP15.

PCR was then utilized to secure the respective receptor sequences for fusion within the Gsα universal vector disclosed above, using the following protocol for each: 100 ng cDNA for RUP15 was added to separate tubes containing 20 of each primer (sense and anti-sense), 3 μL of 10 mM dNTPs, 10 μL of 10× TaqPlus™ Precision buffer, 1 μL of TaqPlus™ Precision polymerase (Stratagene: #600211), and 80 μL of water. Reaction temperatures and cycle times for RUP15 were as follows with cycle steps 2 through 4 were repeated 35 times: 94° C. for 1 min; 94° C. for 30 seconds; 62° C. for 20 sec; 72° C. 1 min 40 sec; and 72° C. 5 min. PCR product for was run on a 1% agarose gel and then purified (data not shown). The purified product was digested with XbaI and EcoRV and the desired inserts purified and ligated into the Gs universal vector at the respective restriction site. The positive clones was isolated following transformation and determined by restriction enzyme digest; expression using 293 cells was accomplished following the protocol set forth infra. Each positive clone for RUP15-Gs Fusion Protein was sequenced to verify correctness. (See, SEQ.ID.NO.:99 for nucleic acid sequence and SEQ.ID.NO.:100 for amino acid sequence).

RUP15 couples via Gs. For the following exemplary GPCR Fusion Proteins, fusion to Gsα was accomplished.

A RUP15-Gsα Fusion Protein construct was made as follows: primers were designed as follows:

(SEQ.ID.NO.: 101; sense)
5′-TCTAGAATGACGTCCACCTGCACCAACAGC-3′
(SEQ.ID.NO.: 102; antisense)
5′-gatatcGCAGGAAAAGTAGCAGAATCGTAGGAAG-3′.

Nucleotides in lower caps are included as spacers in the restriction sites between the G protein and RUP15. The sense and anti-sense primers included the restriction sites for EcoRV and XbaI, respectively, such that spacers (attributed to the restriction sites) exists between the G protein and RUP15.

PCR was then utilized to secure the respective receptor sequences for fusion within the Gsα universal vector disclosed above, using the following protocol for each: 100 ng cDNA for RUP15 was added to separate tubes containing 41 of each primer (sense and anti-sense), 3 μL of 10 mM dNTPs, 10 μL of 10× TaqPlus™ Precision buffer, 1 uL of TaqPlus™ Precision polymerase (Stratagene: #600211), and 80 μL of water. Reaction temperatures and cycle times for RUP15 were as follows with cycle steps 2 through 4 were repeated 35 times: 94° C. for 1 min; 94° C. for 30 seconds; 62° C. for 20 sec; 72° C. 1 min 40 sec; and 72° C. 5 min. PCR product for was run on a 1% agarose gel and then purified (data not shown). The purified product was digested). The purified product was digested with EcoRV and XbaI and the desired inserts purified and ligated into the Gs universal vector at the respective restriction site. The positive clones was isolated following transformation and determined by restriction enzyme digest; expression using 293 cells was accomplished following the protocol set forth infra. Each positive clone for RUP15-Gs Fusion Protein was sequenced to verify correctness. (See, SEQ.ID.NO.:103 for nucleic acid sequence and SEQ.ID.NO.:104 for amino acid sequence).

b. Gq(6 Amino Acid Deletion)/Gi Fusion Construct

The design of a Gq (del)/Gi fusion construct can be accomplished as follows: the N-terminal six (6) amino acids (amino acids 2 through 7, having the sequence of TLESIM (SEQ.ID.NO.: 129) Gαq-subunit will be deleted and the C-terminal five (5) amino acids, having the sequence EYNLV (SEQ.ID.NO.:130) will be replace with the corresponding amino acids of the Gαi Protein, having the sequence DCGLF (SEQ.ID.NO.:131). This fusion construct will be obtained by PCR using the following primers:

(SEQ.ID.NO.: 132)
5′-gatcaagcttcCATGGCGTGCTGCCTGAGCGAGGAG-3′
and
(SEQ.ID.NO.: 133)
5′-gatcggatccTTAGAACAGGCCGCAGTCCTTCAGGTTCAGCTGCAGG
ATGGTG-3′

and Plasmid 63313 which contains the mouse Gαq-wild type version with a hemagglutinin tag as template. Nucleotides in lower caps are included as spacers.

TaqPlus Precision DNA polymerase (Stratagene) will be utilized for the amplification by the following cycles, with steps 2 through 4 repeated 35 times: 95° C. for 2 min; 95° C. for 20 sec; 56° C. for 20 sec; 72° C. for 2 min; and 72° C. for 7 min. The PCR product will be cloned into a pCRII-TOPO vector (Invitrogen) and sequenced using the ABI Big Dye Terminator kit (P.E. Biosystem). Inserts from a TOPO clone containing the sequence of the fusion construct will be shuttled into the expression vector pcDNA3.1(+) at the HindIII/BamHI site by a 2 step cloning process.

### Example 6

### Tissue Distribution of the Disclosed Human GPCRs: RT-PCR

RT-PCR was applied to confirm the expression and to determine the tissue distribution of several novel human GPCRs. Oligonucleotides utilized were GPCR-specific and the human multiple tissue cDNA panels (MTC, Clontech) as templates. Taq DNA polymerase (Stratagene) were utilized for the amplification in a 40 μl reaction according to the manufacturer's instructions. 20 μl of the reaction will be loaded on a 1.5% agarose gel to analyze the RT-PCR products. Table J below lists the receptors, the cycle conditions and the primers utilized.

TABLE J

| | Cycle | | | | |
|---|---|---|---|---|---|
| | Conditions | | | | |
| | Min (′), Sec (″) | | | | |
| | Cycles 2-4 | | | | |
| Receptor | repeated 30 | 5′ Primer | 3′ Primer | DNA | Tissue |
| Identifier | times | (SEQ.ID.NO.) | (SEQ.ID.NO.) | Fragment | Expression |
| hRUP10 | 94° for 30″ | CATGTATGC | GCTATGCCTG | 730 bp | Kidney, |
| | 94° for 10″ | CAGCGTCCT | AAGCCAGTC | | leukocyte, liver, |
| | 62° C. for 20″ | GCTCC (105) | TTGTG (106) | | placenta and |
| | 72° for 1′ | | | | spleen |
| | 72° for 7′ | | | | |
| | *cycles 2-4 | | | | |
| | repeated 35 times | | | | |
| hRUP11 | 94° for 2′ | GCACCTGCT | CACAGCGCT | 630 bp | Liver, kidney, |
| | 94° for 15″ | CCTGAGCAC | GCAGCCCTG | | pancreas, colon, |
| | 67° C. for 15″ | CTTCTCC | CAGCTGGC | | small intestinal, |
| | 72° for 45″ | (107) | (108) | | spleen and |
| | 72° for 5′ | | | | prostate |
| hRUP12 | 94° for 2′ | CCAGTGATG | CAGACACTT | 490 bp | Brain, colon, |
| | 94° for 15″ | ACTCTGTCC | GGCAGGGAC | | heart, kidney, |
| | 66° C. for 15″ | AGCCTG (109) | GAGGTG (110) | | leukocyte, |
| | 72° for 45″ | | | | pancreas, |
| | 72° for 5′ | | | | prostate, small |
| | | | | | intestinal, |
| | | | | | spleen, testis, |
| | | | | | and thymus |
| hRUP13 | 94° for 1′ | CTTGTGGTCT | CATATCCCTC | 700 bp | Placenta and |
| | 94° for 15″ | ACTGCAGCA | CGAGTGTCC | | lung |
| | 68° C. for 20″ | TGTTCCG | AGCGGC (112) | | |
| | 72° for 1′ 45″ | (111) | | | |
| | 72° for 5′ | | | | |
| hRUP14 | 94° for 1′ | ATGGATCCT | CAAGAACAG | 700 bp | Not yet |
| | 94° for 15″ | TATCATGGC | GTCTCATCTA | | determined |
| | 68° C. for 20″ | TTCCTC (113) | AGAGCTCC | | |
| | 72° for 1′ 45″ | | (114) | | |
| | 72° for 5′ | | | | |
| hRUP16 | 94° for 30″ | CTCTGATGC | GTAGTCCACT | 370 bp | Fetal brain, fetal |
| | 94° for 5″ | CATCTGCTG | GAAAGTCCA | | kidney and fetal |
| | 69° C. for 15″ | GATTCCTG | GTGATCC | | skeletal muscle |
| | 72° for 30″ | (115) | (116) | | |
| | 72° for 5′ | | | | |
| hRUP18 | 94° for 2′ | TGGTGGCGA | GTTGCGCCTT | 330 bp | Pancreas |
| | 94° for 15″ | TGGCCAACA | AGCGACAGA | | |
| | 60° C. for 20″ | GCGCTC (117) | TGACC (118) | | |
| | 72° for 1′ | | | | |
| | 72° for 5′ | | | | |
| hRUP21 | 94° for 1′ | TCAACCTGT | AAGGAGTAG | | Kidney, lung |
| | 94° for 15″ | ATAGCAGCA | CAGAATGGT | | and testis |
| | 56° C. for 20″ | TCCTC (119) | TAGCC (120) | | |
| | 72° for 40″ | | | | |
| | *cycles 2-3 | | | | |
| | repeated 30 times | | | | |
| hRUP22 | 94° for 30″ | GACACCTGT | CTGATGGAA | | Testis, thymus |
| | 94° for 15″ | CAGCGGTCG | GTAGAGGCT | | and spleen |
| | 69° C. for 20″ | TGTGTG (121) | GTCCATCTC | | |
| | 72° for 40″ | | (122) | | |
| | *cycles 2-3 | | | | |
| | repeated 30 times | | | | |
| hRUP23 | 94° for 2′ | GCGCTGAGC | CACGGTGAC | 520 bp | Placenta |
| | 94° for 15″ | GCAGACCAG | GAAGGGCAC | | |
| | 60° C. for 20″ | TGGCTG (123) | GAGCTC (124) | | |
| | 72° for 1′ | | | | |
| | 72° for 5′ | | | | |
| hRUP26 | 94° for 2′ | AGCCATCCC | CCAGGTAGG | 470 bp | Pancreas |
| | 94° for 15″ | TGCCAGGAA | TGTGCAGCA | | |
| | 65° C. for 20″ | GCATGG (125) | CAATGGC | | |
| | 72° for 1′ | | (126) | | |
| | 72° for 5′ | | | | |
| hRUP27 | 94° for 30″ | CTGTTCAAC | ATCATGTCTA | 890 bp | Brain |
| | 94° for 10″ | AGGGCTGGT | GACTCATGGT | | |
| | 55° C. for 20″ | TGGCAAC | GATCC (128) | | |
| | 72° for 1′ | (127) | | | |
| | 72° for 3′ | | | | |
| | *cycles 2-4 | | | | |
| | repeated 35 times | | | | |

### Example 7

### Protocol: Direct Identification of Inverse Agonists and Agonists

A. [³⁵S]GTPγS Assay

Although we have utilized endogenous, constitutively active GPCRs for the direct identification of candidate compounds as, e.g., inverse agonists, for reasons that are not altogether understood, intra-assay variation can become exacerbated. Preferably, then, a GPCR Fusion Protein, as disclosed above, is also utilized with a non-endogenous, constitutively activated GPCR. We have determined that when such a protein is used, intra-assay variation appears to be substantially stabilized, whereby an effective signal-to-noise ratio is obtained. This has the beneficial result of allowing for a more robust identification of candidate compounds. Thus, it is preferred that for direct identification, a GPCR Fusion Protein be used and that when utilized, the following assay protocols be utilized.

1. Membrane Preparation

Membranes comprising the constitutively active orphan GPCR Fusion Protein of interest and for use in the direct identification of candidate compounds as inverse agonists, agonists or partial agonists are preferably prepared as follows:

a. Materials

“Membrane Scrape Buffer” is comprised of 20 mM HEPES and 10 mM EDTA, pH 7.4; “Membrane Wash Buffer” is comprised of 20 mM HEPES and 0.1 mM EDTA, pH 7.4; “Binding Buffer” is comprised of 20 mM HEPES, 100 mM NaCl, and 10 mM MgCl₂, pH 7.4

b. Procedure

All materials will be kept on ice throughout the procedure. Firstly, the media will be aspirated from a confluent monolayer of cells, followed by rinse with 10 ml cold PBS, followed by aspiration. Thereafter, 5 ml of Membrane Scrape Buffer will be added to scrape cells; this will be followed by transfer of cellular extract into 50 ml centrifuge tubes (centrifuged at 20,000 rpm for 17 minutes at 4° C.). Thereafter, the supernatant will be aspirated and the pellet will be resuspended in 30 ml Membrane Wash Buffer followed by centrifuge at 20,000 rpm for 17 minutes at 4° C. The supernatant will then be aspirated and the pellet resuspended in Binding Buffer. This will then be homogenized using a Brinkman Polytron™ homogenizer (15-20 second bursts until the all material is in suspension). This is referred to herein as “Membrane Protein”.

2. Bradford Protein Assay

Following the homogenization, protein concentration of the membranes will be determined using the Bradford Protein Assay (protein can be diluted to about 1.5 mg/ml, aliquoted and frozen (−80° C.) for later use; when frozen, protocol for use will be as follows: on the day of the assay, frozen Membrane Protein is thawed at room temperature, followed by vortex and then homogenized with a polytron at about 12×1,000 rpm for about 5-10 seconds; it was noted that for multiple preparations, the homogenizor should be thoroughly cleaned between homoginezation of different preparations).

a. Materials

Binding Buffer (as per above); Bradford Dye Reagent; Bradford Protein Standard will be utilized, following manufacturer instructions (Biorad, cat. no. 500-0006).

b. Procedure

Duplicate tubes will be prepared, one including the membrane, and one as a control “blank”. Each contained 800 ul Binding Buffer. Thereafter, 10 μl of Bradford Protein Standard (1 mg/ml) will be added to each tube, and 10 μl of membrane Protein will then be added to just one tube (not the blank). Thereafter, 200 ul of Bradford Dye Reagent will be added to each tube, followed by vortex of each. After five (5) minutes, the tubes will be re-vortexed and the material therein will be transferred to cuvettes. The cuvettes will then be read using a CECIL 3041 spectrophotometer, at wavelength 595.

3. Direct Identification Assay

a. Materials

GDP Buffer consisted of 37.5 ml Binding Buffer and 2 mg GDP (Sigma, cat. no. G-7127), followed by a series of dilutions in Binding Buffer to obtain 0.2 μM GDP (final concentration of GDP in each well was 0.1 μM GDP); each well comprising a candidate compound, has a final volume of 200 ul consisting of 100 μl GDP Buffer (final concentration, 0.1 μM GDP), 50 ul Membrane Protein in Binding Buffer, and 50 μl [³⁵S]GTPγS (0.6 nM) in Binding Buffer (2.5 μl [³⁵S]GTPγS per 10 ml Binding Buffer).

b. Procedure

Candidate compounds will be preferably screened using a 96-well plate format (these can be frozen at −80° C.). Membrane Protein (or membranes with expression vector excluding the GPCR Fusion Protein, as control), will be homogenized briefly until in suspension. Protein concentration will then be determined using the Bradford Protein Assay set forth above. Membrane Protein (and control) will then be diluted to 0.25 mg/ml in Binding Buffer (final assay concentration, 12.5 μg/well). Thereafter, 100 μl GDP Buffer was added to each well of a Wallac Scintistrip™ (Wallac). A 5 μl pin-tool will then be used to transfer 5 μl of a candidate compound into such well (i.e., 5 μl in total assay volume of 200 μl is a 1:40 ratio such that the final screening concentration of the candidate compound is 10 μM). Again, to avoid contamination, after each transfer step the pin tool should be rinsed in three reservoirs comprising water (1×), ethanol (1×) and water (2×)—excess liquid should be shaken from the tool after each rinse and dried with paper and kimwipes. Thereafter, 50 μl of Membrane Protein will be added to each well (a control well comprising membranes without the GPCR Fusion Protein was also utilized), and pre-incubated for 5-10 minutes at room temperature. Thereafter, 50 μl of [³⁵S]GTPγS (0.6 nM) in Binding Buffer will be added to each well, followed by incubation on a shaker for 60 minutes at room temperature (again, in this example, plates were covered with foil). The assay will then be stopped by spinning of the plates at 4000 RPM for 15 minutes at 22° C. The plates will then be aspirated with an 8 channel manifold and sealed with plate covers. The plates will then be read on a Wallace 1450 using setting “Prot. #37” (as per manufacturer instructions).

B. Cyclic AMP Assay

Another assay approach to directly identified candidate compound was accomplished by utilizing a cyclase-based assay. In addition to direct identification, this assay approach can be utilized as an independent approach to provide confirmation of the results from the [³⁵S]GTPγS approach as set forth above.

A modified Flash Plate™ Adenylyl Cyclase kit (New England Nuclear; Cat. No. SMP004A) was preferably utilized for direct identification of candidate compounds as inverse agonists and agonists to constitutively activated orphan GPCRs in accordance with the following protocol.

Transfected cells were harvested approximately three days after transfection. Membranes were prepared by homogenization of suspended cells in buffer containing 20 mM HEPES, pH 7.4 and 10 mM MgCl₂. Homogenization was performed on ice using a Brinkman Polytron™ for approximately 10 seconds. The resulting homogenate is centrifuged at 49,000×g for 15 minutes at 4° C. The resulting pellet was then resuspended in buffer containing 20 mM HEPES, pH 7.4 and 0.1 mM EDTA, homogenized for 10 seconds, followed by centrifugation at 49,000×g for 15 minutes at 4° C. The resulting pellet was then stored at −80° C. until utilized. On the day of direct identification screening, the membrane pellet as slowly thawed at room temperature, resuspended in buffer containing 20 mM HEPES, pH 7.4 and 10 mM MgCL2, to yield a final protein concentration of 0.60 mg/ml (the resuspended membranes are placed on ice until use).

cAMP standards and Detection Buffer (comprising 2 μCi of tracer [¹²⁵I cAMP (100 μl] to 11 ml Detection Buffer) were prepared and maintained in accordance with the manufacturer's instructions. Assay Buffer was prepared fresh for screening and contained 20 mM HEPES, pH 7.4, 10 mM MgCl₂, 20 mM phosphocreatine (Sigma), 0.1 units/ml creatine phosphokinase (Sigma), 50 μM GTP (Sigma), and 0.2 mM ATP (Sigma); Assay Buffer was then stored on ice until utilized.

Candidate compounds identified as per above (if frozen, thawed at room temperature) were added, preferably, to 96-well plate wells (3 μl/well; 12 μM final assay concentration), together with 40 μl Membrane Protein (30 μg/well) and 50 μl of Assay Buffer. This admixture was then incubated for 30 minutes at room temperature, with gentle shaking.

Following the incubation, 100 μl of Detection Buffer was added to each well, followed by incubation for 2-24 hours. Plates were then counted in a Wallac MicroBeta™ plate reader using “Prot. #31” (as per manufacturer instructions).

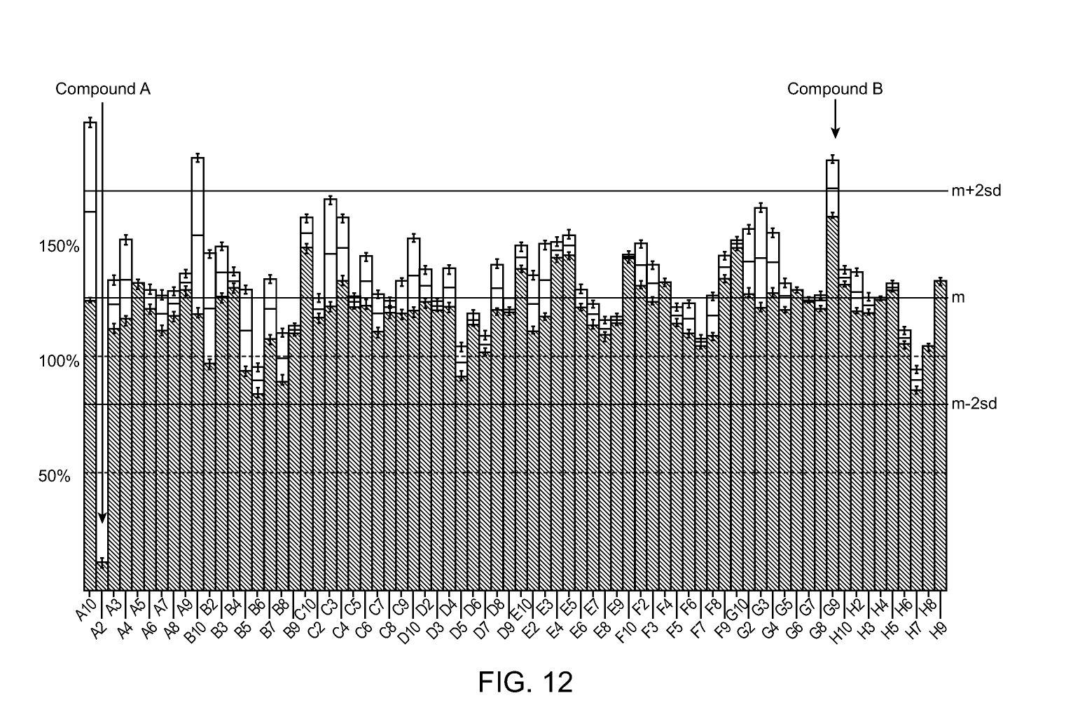

A representative screening assay plate (96 well format) result is presented in FIG. 12. Each bar represents the results for a different compound in each well, plus RUP13-Gsα Fusion Protein construct, as prepared in Example 5(a) above. The representative results presented in FIG. 12 also provide standard deviations based upon the mean results of each plate (“m”) and the mean plus two arbitrary preference for selection of inverse agonists as “leads” from the primary screen involves selection of candidate compounds that that reduce the percent response by at least the mean plate response, minus two standard deviations. Conversely, an arbitrary preference for selection of an agonists as “leads” from the primary screen involves selection of candidate compounds that increase the percent response by at least the mean plate response, plus the two standard deviations. Based upon these selection processes, the candidate compounds in the following wells were directly identified as putative inverse agonist (Compound A) and agonist (Compound B) to RUP13 in wells A2 and G9, respectively. See, FIG. 12. It is noted for clarity: these compounds have been directly identified without any knowledge of the endogenous ligand for this GPCR. By focusing on assay techniques that are based upon receptor function, and not compound binding affinity, we are able to ascertain compounds that are able to reduce the functional activity of this receptor (Compound A) as well as increase the functional activity of the receptor (Compound B). Based upon the location of these receptor in lung tissue (see, for example, hRUP13 and hRUP21 in Example 6), pharmaceutical agents can be developed for potential therapeutic treatment of lung cancer.

References cited throughout this patent document, including co-pending and related patent applications, unless otherwise indicated, are fully incorporated herein by reference. Modifications and extension of the disclosed inventions that are within the purview of the skilled artisan are encompassed within the above disclosure and the claims that follow.

Although a variety of expression vectors are available to those in the art, for purposes of utilization for both the endogenous and non-endogenous human GPCRs, it is most preferred that the vector utilized be pCMV. This vector was deposited with the American Type Culture Collection (ATCC) on Oct. 13, 1998 (10801 University Blvd., Manassas, Va. 20110-2209 USA) under the provisions of the Budapest Treaty for the International Recognition of the Deposit of Microorganisms for the Purpose of Patent Procedure. The DNA was tested by the ATCC and determined to be viable. The ATCC has assigned the following deposit number to pCMV: ATCC #203351.

1-80. (canceled)

81. An isolated polynucleotide comprising a nucleic acid encoding a G protein-coupled receptor (GPCR) comprising an amino acid sequence having at least about 80% identity to an amino acid sequence selected from the group consisting of: SEQ ID NO:2; SEQ ID NO:4; SEQ ID NO:6; SEQ ID NO:8; SEQ ID NO:10; SEQ ID NO:12; SEQ ID NO:14; SEQ ID NO:16; SEQ ID NO:18; SEQ ID NO:20; SEQ ID NO:22; SEQ ID NO:24; SEQ ID NO:26; SEQ ID NO:28; SEQ ID NO:30; SEQ ID NO:32; SEQ ID NO:34; SEQ ID NO:36; SEQ ID NO:38; and SEQ ID NO:40.

82. An isolated polynucleotide according to claim 81, wherein said nucleic acid comprises a nucleotide sequence selected from the group consisting of: SEQ ID NO:1; SEQ ID NO:3; SEQ ID NO:5; SEQ ID NO:7; SEQ ID NO:9; SEQ ID NO:11; SEQ ID NO:13; SEQ ID NO:15; SEQ ID NO:17; SEQ ID NO:19; SEQ ID NO:21; SEQ ID NO:23; SEQ ID NO:25; SEQ ID NO:27; SEQ ID NO:29; SEQ ID NO:31; SEQ ID NO:33; SEQ ID NO:35; SEQ ID NO:37; and SEQ ID NO:39.

83. A vector comprising a polynucleotide according to claim 81.

84. A vector according to claim 83, wherein said vector is an expression vector and wherein the polynucleotide is operably linked to a promoter.

85. A recombinant host cell comprising a vector according to claim 83.

86. A recombinant host cell comprising an expression vector according to claim 84.

87. A process for making a recombinant host cell comprising:
(a) introducing an expression vector according to claim 84 into a suitable host cell; and
(b) culturing the host cell under conditions that allow for expression of said G protein-coupled receptor by said cell.

88. An isolated membrane of a recombinant host cell according to claim 87, wherein the isolated membrane comprises the G protein-coupled receptor.

89. An isolated G protein-coupled receptor comprising an amino acid sequence having at least about 80% identity to an amino acid sequence selected from the group consisting of: SEQ ID NO:2; SEQ ID NO:4; SEQ ID NO:6; SEQ ID NO:8; SEQ ID NO:10; SEQ ID NO:12; SEQ ID NO:14; SEQ ID NO:16; SEQ ID NO:18; SEQ ID NO:20; SEQ ID NO:22; SEQ ID NO:24; SEQ ID NO:26; SEQ ID NO:28; SEQ ID NO:30; SEQ ID NO:32; SEQ ID NO:34; SEQ ID NO:36; SEQ ID NO:38; and SEQ ID NO:40.

90. A method comprising:
(a) contacting a candidate compound with a recombinant host cell or membrane thereof comprising a G protein-coupled receptor according to claim 89; and
(b) measuring the ability of the compound to inhibit or stimulate the G protein-coupled receptor.
